(19) United States
(12) Patent Application Publication
(10) Pub. No.: US 2022/0333130 A1
(43) Pub. Date: Oct. 20, 2022

(54) Episomal expression, genomic integrated lentiviral vector expression and mRNA expression of Potent Immunoglobulins Including Dimeric Immunoglobulin A1 and A2 via a furin cleavage site and 2A self-processing peptide to Enable Mucosal and Hematological Based Immunity or Protection via Gene Therapy for Allergens, viruses, HIV, bacteria, infections, pathology associated proteins, systemic pathologies, cancer, toxins and unnatural viruses.

(71) Applicant: Roger B. Swartz, Clifton Park, NY (US)

(72) Inventor: Roger B. Swartz, Clifton Park, NY (US)

(21) Appl. No.: 17/227,372

(22) Filed: Apr. 11, 2021

(51) Int. Cl. C12N 15/86; A61K 48/00; C07K 16/18

(52) U.S. Cl. CPC C12N 15/86; A61K 48/005; C07K 16/18; C12N 2830/48; C12N 2830/50; C12N 2840/203; C12N 2740/15043

(57) ABSTRACT

The present invention contemplates mRNA, episomal and retroviral genomic gene therapy based short-term, intermediate or long-term vaccine, immunization, protection or therapy—that can also be administered as a retroviral genomic gene therapy—method to provide mucosal and hematological protection to humans to protect against pandemic and non-pandemic viruses, bacterial infections, fungi, allergens or the cause of allergic reactions, systemic pathological conditions, cancer and anti-biowarfare agents (e.g. natural and unnatural viruses and toxins) where mucosal immunity and potentially hematological immunity is achieved through mRNA, episomal or genomic expression of dimeric immunoglobulin A1 (dIgA1) and dimeric immunoglobulin A2 (dIgA2). The present invention provides methods, immunoglobulin compositions and vector constructs to express potent immunoglobulins that are derived from human blood of a human currently infected with, affected by, exposed to or recovered from any of a wide range of allergens or the cause of allergic reactions, pathogens (including, viruses, virus mutants, bacterial infections and fungi) and systemic pathological ailments (including cancer and other disorders), developed from phage display technology or mice or other animals with a humanized immune systems, transgenic mice or chimeric antibodies a fusion of non-human vetebrates (e.g. mouse or rabbit) and human. The immunoglobulin compositions include the heavy chain variable, diversity and joining (VDJ or Variable Heavy Region genes) segment immunoglobulin DNA and/or polypeptide sequence from humans identified to have developed high affinity immunoglobulins against the antigen, protein or proteins of interest and either to use the exact immunoglobulin heavy chain and light chain polypeptide sequences identified from the memory B-cell that produced them or to modify or engineer some of the immunoglobulin heavy chain and light chain constant domains to reduce, change or modulate effector functions. Although, ideally there are no changes made to the immunoglobulins light and heavy chains as identified from the memory B-cell that produced them. Modification may occur at the Hinge region, Constant Heavy 2 (C_{H}2) domain and Constant Heavy 3 (C_{H}3) domain for the immunoglobulin heavy chain polypeptide with optional modification or change of Constant Heavy 1 (C_{H}1), optional modification or change constant light (C_{L}) chain domain. The resulting antibodies can either be used as a monoclonal or antibody cocktail of (Immunoglobulin Class G subclass1) IgG1, IgG2, IgG3 and other subclasses, IgA1 monomer and IgA2 monomer and dimeric IgA1 (dIgA1) and dimeric IgA2 (dIgA2) immunoglobulins (as identified by the binding affinity of B-cells that expressed immunoglobulins are coded for as necessary to represent the binding affinity (e.g. such as based on complementarity determining Regions (CDRs) or V-regions) in the monoclonal or antibody cocktail). Alternatively, combinatorial libraries of single chain variable fragments (scFv) may be generated from human B-cells or other animal B-cells that may or may not have been exposed to the allergen, pathogen, cancer, or pathological ailment, or suspected or identified biowarfare agent or protein where phage display technology and mutagenesis can be used to identify potent V_{H} and V_{L} immunoglobulin fragments that can be incorporated into full-length immunoglobulin heavy and light chains incorporated into vectors for mRNA expression, episomal expression or retroviral gene delivery (retroviral insertion into genomic DNA) based gene-therapy. Further, mice or other animals can also achieve humanized immune system by implanting human hematopoietic progenitor cells into the animal or transplanting human fetal thymus, liver and bone marrow into mice or other animals where exposure to antigens, allergens or other foreign and non-foreign proteins can result in an adaptive immune response and potential affinity maturation. Additionally, transgenic mice where human immunoglobulin (Ig) genes are inserted into the genome to replacing the endogenous Ig genes making the mice or other non-human vertebrate such as rabbits or hamsters capable of producing fully human antibodies from exposure to antigen may be used to identify potent immunoglobulins. Non-human vetebrates (e.g. mouse or rabbit) may be used to identify potent immunoglobulin binding regions or potent immunoglobulin complementarity determining regions (CDRs) for fusion with human antibodies giving rise to chimeric antibodies. The identified immunoglobulins from these methods may be further optimized through mutagenesis techniques and will be expressed in the recipient via mRNA, via an episome or via retroviral insertion into their genomic DNA of the cells of interest to be expressed via intramuscular administration, intravenous administration, endoscopy based administration to the lamina propria of the stomach and/or small intestine, via ingestion or administration proximal to lymph nodes. Preferred cells to target to receive the vector include muscle cells, liver cells especially hepatocytes and B-cells including memory B-cells, Germinal Center B-cells, memory plasma B-cells, a plasma blast, and naïve B-cells. The vector will be ideally delivered as a naked vector, in a vesicle based delivery system such as a lipid nano-particle, in a recombinant Adeno Associated Virus (rAAV) with preference for AAV serotype 8 (AAV8) containing a single-stranded Deoxyribonucleic acid (ssDNA), an adenovirus delivery system, a lentivirus delivery system, lentiviral mRNA delivery via mutated reverse transcriptase protein, lentiviral retroviral vector or mRNA delivery via mutated integrase protein, or a vesicle-based delivery system using mRNA, single-stranded DNA or double-stranded DNA. When designing an mRNA, AAV viral vector, adenovirus vector, integration incompetent lentivirus vector or lentivirus retroviral vector, encoding for dIgA1 or dIgA2 a single vector will code for the entire immunoglobulin and J chain (Joining Chain) expression for dIgA1 or dIgA2 where expression may occur with a single start codon and stop codon for each transgene and in some embodiments a second start codon for J chain expression. The use of a single start and stop codon is enabled by placing in the 5′ to 3′ direction a furin cleavage site concomitantly followed by a 2A self-processing peptide or furin cleavage site only between each gene of any number of consecutive transgenes as a single open reading frame. Further, in some embodiments MZB1 will optionally be encoded in the mRNA, viral, retroviral or non-viral vectors (See FIGS. 13, 15, 16, 17, 18, 19, 20 as examples). The specific DNA of the human donor can be identified as follows: Cluster of Differentiation 27+(CD27+) IgG+ and CD27+ IgA+ memory B-cells, other memory B-cells, or plasmablast B-cells and even potentially memory plasma B-cells will be isolated from blood using established methods. Each resulting isotype of memory B-cell or together will be subjected to a competitive binding assay using magnetic pull down and Fluorescence Activated Cell Sorting (FACS) methods to identify the memory B-cells with the greatest binding affinity to the virus, bacteria, antigen, allergens, self-antigen, pathogenic protein, or other foreign and non-foreign bodies and proteins of interest. Isolated CD27+ or other Cluster of Differentiation memory B-cells will use well-established methods to identify the genetic sequence and in turn the polypeptide sequence of the immunoglobulin heavy and light chains of the cell surface IgG+ or IgA+ receptor. Immunoglobulin mRNA or DNA will be incorporated into vector construct coding for antibodies to be evaluated for binding affinity and safety in addition to modifying them in a variety of ways as described herein and then to be incorporated into an mRNA vector to enable mRNA based expression or viral or non-viral vector to enable episomal immunoglobulin expression. Alternatively, the lentivirus vectors may be used for episomal expression or as a retroviral vector intended for retroviral integration in the host genomic DNA. Additionally, a method to improve the potency of a vaccine is designed by targeted delivery of antigenic proteins or protein encoding mRNA to the lamina propria of the respiratory tract or gastrointestinal tract.

### CROSS-REFERENCE TO RELATED APPLICATIONS

This present application claims benefit to U.S. provisional patent application Ser. No. 63/008,844 filed on Apr. 13, 2020 and 63/076,527 filed on Sep. 10, 2020. The entire disclosure is included herein in its entirety at least by reference.

### BACKGROUND OF THE INVENTION

1 Field of the Invention

The present invention is in the field of healthcare biotechnology and pertains particularly to a therapeutic gene therapy engendering mRNA, episomal or genomic expression via lentiviral vector integration into genomic DNA of dimeric immunoglobulin A1 (dIgA1), dIgA2 and engineered variants against Viruses, Allergens, Fungi, HIV, H. pylori other bacterial infections, systemic pathologies, IgE, cytokines, cancerous tumors or as anti-biowarfare protection (e.g. protection from purposefully spread natural and unnatural viruses and toxins). This invention includes a method to encode for dimeric immunoglobulin A (dIgA1 or dIgA2) and engineered variants (such as dIgA with modified hinge lengths or other modified constant regions) on a vector as a means to deliver a gene therapy to humans. Specifically, dIgA (dIgA1, dIgA2 or engineered variants) encoded for on a single vector or multiple vectors is the invention. dIgA may be encoded for on a single DNA, or RNA with the use of a furin cleavage site and 2A self-processing peptide between consecutive transgenes. The transgenes that make up dIgA include (1) the immunoglobulin heavy chain, (2) the immunoglobulin light chain and (3) J Chain. These three proteins may be encoded for a on a single genetic element in one or two open reading frames. Where all the transgenes in any one open reading frame will have in the 5′ to 3′ direction a furin cleavage site followed by a 2A self-processing peptide placed between any two adjacent and consecutive transgenes. The use of a 2A self-processing peptide allows for efficient cleavage points between the consecutive transgenes completely removing any trace of the 2A self-processing peptide in the final immunoglobulin. In one embodiment MZB1 may also be encoded for by the vector. In another embodiment mRNA encodes for dIgA (dIgA1, dIgA2 or engineered variants) over one or more mRNA vectors. In an alternative configuration, a 2A self-processing peptide will be used between consecutive transgenes in a single open reading frame in the absence of the furin cleavage site leaving the 2A residue on the C-terminal end of the protein encoded for by the gene that is directly 5′ of the 2A gene. In another configuration mRNA encodes for dIgA (dIgA1, dIgA2 or engineered variants) over one or more mRNA vectors.

In the invention dimeric immunoglobulin A1 (dIgA1), dIgA2 and engineered variants specific against Viruses, Allergens, Fungi, HIV, H. pylori other bacterial infections, systemic pathologies, IgE, cytokines, cancerous tumors or as anti-biowarfare protection may have such binding affinity of dIgA and engineered variants will be based on one or more of individuals affected by such ailments, developed by Phage Display Technology with mutagenesis techniques, discovered and optimized from mice or other animals with humanized immune systems, derived from transgenic mice or derived non-human vertebrate (e.g. mouse or rabbit) intended for fusion with human antibodies giving rise to chimeric antibodies. Further a method to improve the potency of a vaccine is designed by targeted delivery of antigenic proteins or mRNA encoding for antigenic proteins to the lamina propria of the respiratory tract or gastrointestinal tract.

For immunoglobulin encoded vectors the entire immunoglobulin genetic sequence would be derived or identified from one of several sources including plasmablasts, CD27+ memory B-cells—expressing immunoglobulins with binding affinities specific for an antigen, virus antigen, allergen, foreign protein, pathogenic protein or cancer related protein—from humans that have been exposed to such antigens or allergens, mice or other animals with a humanized immune systems, non-human vetebrates (e.g. mouse or rabbit) intended for chimeric antibodies, transgenic mice or combinatorial libraries that may use phage display technology and mutagenesis. Those B-cells would be CD27+ or other CD+ IgG+ and IgA+ memory B-cells or plasmablasts B-cells that bear cell surface immunoglobulins easily allowing for their isolation based on binding affinity for an antigen, allergen, foreign protein, native protein, or pathogen related protein of interest through competitive binding cell sorting methods such as flow cytometry or fluorescence activated cell sorting (FACS) or competitive binding magnetic pull down methods that incorporate magnetic beads biotinylated to an antigen, allergen or foreign protein of interest and a competing protein for the antigen. Plasmids may be created that encode for those immunoglobulins to be further evaluated in addition to subjecting them to targeted mutagenesis. Alternatively, combinatorial libraries may be generated and phage display technology can be used to identify potent single chain variable fragments (scFv) that can be used to identify potent V_{H} and V_{L} pairs of high avidity and binding affinity that can be incorporated into a plasmid expressing a full length immunoglobulin heavy and light chains for evaluation. Other models such as non-human vetebrates (e.g. mouse or rabbit) intended for chimeric antibodies, transgenic mice or mice or other animals with humanized immune systems may be used to identify potent immunoglobulins or potent binding regions. Additionally, the V-region DNA of the heavy (IgH) and light chain (IgL) of the immunoglobulins will be incorporated into a vector construct that could use either the constant region DNA identified in humanized or transgenic mice, filamentous phage or B-cell expressing the identified immunoglobulin of interest or another source of genetic information that may be engineered may replace all or part of the constant region DNA that may also include isotype switching of constant region genetic information, mixes of two constant regions from two isotypes that may be accomplished with combinations of Fab and natural or engineered Fc domains or F(ab′)2 and natural or engineered pFc′ domains. The mRNA, episome, or retroviral incorporation of the DNA via lentivirus delivery system may be delivered to B-cells, muscle cells, liver cells (such as hepatocytes), any human cell, endoscopy based delivery to the lamina propria of the lungs, stomach and/or small intestine destined for memory B-cells in the lamina propria and the supporting lymph nodes as well as other quiescent cells potentially. B-cells targeted to receive the gene therapy may include memory B-cells, Germinal Center B-cells, memory plasma B-cells, a plasma blast, and naïve B-cells The episome may be delivered to the cells of interest via an adeno-associated virus, adenovirus, lentivirus based deliver system or via a vesicle based delivery system such as a lipid nano particle as one of many examples. Additionally, in other embodiments the reinvigoration of an FDA approved antibody or the repurposing of antibodies by placing their V-regions in a vector encoding for dIgA is part of the present invention. Further, in other embodiments a method to improve the potency of a vaccine is designed by targeted delivery of antigenic proteins or protein encoding mRNA to the lamina propria of the respiratory tract or gastrointestinal tract.

### 2. Discussion of the State of the Art

The primary mode of infection, carcinogen exposure and allergen entry for the vast majority of carcinogens, allergens, pathogens such as viruses, bacteria affecting the lungs, including gut bacteria, viruses and other disease causing foreign bodies is through our mucosal barriers. Similarly, the primary way that allergens such as fungi, dust mite feces, peanuts, tree nuts and pollen cause allergic reactions is also through our mucosal barriers especially the respiratory tract and in some cases the digestive tracts. Cancer has a specific term for cancers affecting the mucosal or epithelial and exocrine barriers referred to as carcinomas. Carcinomas make up 80%-90% of all cancer diagnosis. Thus, it is safe to say that if mankind had effective means of mucosal protection against infectious agents, allergens or unnatural viruses and toxins that their transmission and thus their allergenic—with respect to but not limited to Type I Hypersensitivity—and pathogenic consequences will be nearly eliminated. With exception to the heart, nearly, every organ is involved in the processing of a liquid or gas that is transported through a lumen or duct Part of or all of these lumens and ducts are aligned with stratified and pseudo stratified columnar epithelial cells that with polymeric immunoglobulin secreting receptor (pIgR) actively transport dimeric immunoglobulin A (dIgA) from the lamina propria to the mucosa or from the surrounding tissue of the exocrine gland to the exocrine duct where upon entering the mucosa or duct of these organs and glands dIgA is converted into secretory immunoglobulin A (SIgA). There may also be some dIgA that is passively diffused to the mucosa and exocrine ducts at relatively low levels in comparison to active transport of dIgA by pIgA. Our mucosal barriers includes but are not limited to our respiratory tract, that is the entirety of the pathways where air flows to the alveoli in the lungs, our digestive tract where food and liquids flow and are absorbed and our reproductive tract, our urinary tract and also our skin.

In order for an individual's immune system to effectively prevent infection from foreign bodies and also avert an allergic reaction to foreign bodies mucosal immunity via immunoglobulins is required. Secretory immunoglobulin A (SIgA1 and SIgA2) is by far the most prevalent of antibodies in the human body and represents a major mode of defense in mucosal immunity. SIgA1 and SIgA2 are made from dimeric immunoglobulin A1 (dIgA1) and dimeric Immunoglobulin A2 (dIgA2) respectively and dIgA1 and dIgA2 is based on Immunoglobulin A1 (IgA1) and Immunoglobulin A2 (IgA2) respectively. Potent IgA1 s and IgA2s as well as IgA variants such as IgAs with varying hinge lengths or modifications to their constant regions can be identified from CD27+ or other CD+ IgA memory B-cells, the V regions of CD27+ or other CD+ IgG memory B cells, other CD memory B-cells, plasmablasts B-cells, from mice or other non-human vertebrates with engineered immune systems or humanized immune systems, from mice or non-human vertebrates B-cells or may have their binding regions discovered via phage display technology such as in the single chain variable fragment or may be discovered from mice or other animals with humanized immune systems.

Expression of dIgA1 and dIgA2 and derivatives of dIgA1 and dIgA2 from mRNA, an episome or genomic DNA via retroviral incorporation of retroviral vector such as one delivered by a lentivirus is claimed in this instant patent. The conversion of dIgA (dIgA1 or dIgA2) to secretory immunoglobulin A (SIgA1 or SIgA2) is a natural process that is part of active transport to the mucosa in humans and is described in FIG. 2. Thus, mucosal immunity depends on two events the production of dIgA and its conversation to SIgA. Further, it is mucosal immunity provided by SIgA is required to greatly reduce the probability of infection and allergic reactions from foreign bodies. It is mucosal immunity provided by SIgA that is required to prevent foreign bodies from crossing the epithelial barriers that line nearly all of the mucosa. It is mucosal immunity provided by SIgA that can effectively prevent humans that get exposed to an infectious agent from passing it on to others. It is mucosal immunity provided by SIgA can eliminate the possibility of antibody-dependent enhancement of infection (ADE) and cytokine storm. In other words, to stop the spread and harmful effects of pathogens, cancers, bacteria, infectious agents and to protect against on all members of society including those at risk mucosal immunity by SIgA is required. Similarly, to stop the harmful effects of allergens especially as related to Type 1 Hypersensitivity on those capable of allergic reactions mucosal immunity by SIgA is required. dIgA is actively transported to the mucosa via polymeric immunoglobulin secreting receptor (pIgR) where upon binding pIgR that is located at the basolateral face of most epithelial cells dIgA is actively transported to the mucosa of the organ or ductile region of the exocrine gland and upon reaching the apical face of the epithelial cell pIgR is cleaved and releases secretory component onto dIgA where a cysteine bond has formed between secretory component and dIgA to become secretory immunoglobulin A (SIgA) (see FIG. 2). (For a review on mucosal immunity see e.g., Pilette, C., et. al., 2001, European Respiratory Journal, 18:571-588., Terauchi, Y., et. al., 2018, Human vaccines & immunotherapeutics, 14:1351-1361.)

Monoclonal antibodies have enjoyed widespread success in their efficacy against a host of ailments. Monoclonal antibodies further enjoy exquisite specificity to their targets as a result of the virtually limitless possible combinations of amino acids that make up their binding regions especially the CDRs and their large binding region surface area that allow binding specificity to be dependent on a large portion of the target surface topology including Van der Waals interactions, surface charge distribution, dipole-dipole interactions, salt bridges, and surface hydrogen bonding. FDA approved monoclonal antibodies that are fully representative of human immunoglobulins are almost universally based on Immunoglobulin class G (IgG) constant regions (when they have constant regions) as attributed to their long half-life of about 21 days that can be increased through modifications of their fragment crystallizable (Fc) regions. Antibody cocktails that are mixtures of 2 or more antibodies have recently received emergency authorization by the FDA as well for their application against the 2019 novel Coronavirus (COVID-19) and mutant forms. One challenge of direct administration of the antibody is the required repeated administration of such antibodies to sustain any benefit derived from them. In addition they can be very costly to produce making their cost sometimes the factor that prevents their administration in lieu of a lower cost and treatment that may be less effective or have more side effects.

Providing people with the genetic instructions or a gene therapy to make the antibodies with their own molecular machinery overcomes the problem of repeated administration in the case of retroviral insertion of the gene encoding for the antibody or makes administration much more long-lived in the case of episomal or even retroviral integration based administration of the genes encoding for the antibody. Additionally, encoding for antibodies through gene therapy overcomes the high cost associated with manufacturing them. Although, as attributed to their longer half-lives IgG based antibodies have been widely prevalent in absence of a gene therapy encoding for them. IgA and dIgA have half-lives of 6 days making them require frequent administrations in their protein form and has been the reason that life sciences companies have not conducted clinical trials with IgA immunoglobulins. And while IgG can reach the mucosa of the lungs they may only do so via passive transport that results in very low levels of IgG in the mucosa while also being more rapidly degraded at a rate faster than that of SIgA in the harsh mucus environment. On the other hand, IgG does not reach the mucosa of the stomach or small intestine making it impossible to derive any significant benefit from them for purposes of blocking infectious agents or allergens from entering the lamina propria of the stomach or small intestine. Further, IgG does not reach the mucosal linings and ductal linings of exocrine glands at high levels as it relies on passive diffusion to those regions. Gene therapy encoded episomal or genomic expression via integration competent lentivirus delivery systems of dIgA solves the challenged associated with their short half-lives and solves the challenge of achieving therapeutically relevant levels of antibodies in the mucosa. In some cases the time period needed for effective immune protection is rather short and also the speed with which antibodies are need is rather fast as in the case of pneumonia where mRNA based administration of the gene therapy can be effective.

Immunoglobulins may be engineered to extend their half-lives or reduce antibody dependent enhancement of infection (ADE). Typically when IgG binds to its target in the mucosa of the lungs it may result in antibody dependent enhancement of infection (ADE) when either the IgG antibodies lack high binding affinities or when antibodies decline in numbers. Immunoglobulins may be engineered to reduce receptor binding to the fragment crystallizable region (Fc) reducing Antibody Dependent Enhancement (ADE) of infection that can result in a cytokine storm type reaction where the resulting immune reactions could cause damage to the host tissue. (See e.g., Iwasaki, A. & Yang, Y., 2020, Nat. Rev. Immunol. 20:339-341.). Further, IgG and all subclasses are not privileged to enter the mucosa of the stomach and intestines and enter the mucosa of the lungs and other tissues at much lower levels than dIgA1 as a result of their reliance on passive diffusion to those areas where dIgA on the other hand is actively transported to the mucosa via polymeric immunoglobulin secreting receptor (pIgR).

Unquestionably, due to its active transport to the mucosa dIgA that becomes SIgA upon active transport to the mucosal or ductal linings is a privileged immunoglobulin to provide mucosal protection over other immunoglobulins such as immunoglobulin class G (IgG) and its 4 classes (IgG1, IgG2, IgG3 and IgG4) as they offer numerous advantages over IgG. Those advantages include two distinct binding faces that allows dIgA and SIgA to agglutinate foreign bodies together preventing their escape, more potent binding affinities over IgGs with as much as 2 orders of magnitude greater binding affinity than IgGs with the same V regions, active and unidirectional transport to the mucosa via binding to polymeric immunoglobulin secreting receptor (pIgR) at the basolateral face of the epithelium, their high production from localized B-cells in the lamina propria, resistance to degradation in the mucosa as part of the conversion of dIgA to SIgA at the conclusion of active transport to the mucosa, dIgA is specifically and naturally tailored for the mucosa, naturally high levels of production at about 5 grams a day, in its SIgA form it has very low cytotoxicity and anti-inflammatory properties in the mucosa, while having both proinflammatory and anti-inflammatory properties in its dIgA form e.g. in the submucosa (See e.g. Bakema, J., van Egmond, M., 2011, Mucosal Immunol, 4:612-624) protecting against cases of a damaged epithelium in the lamina propria and bloodstream, and SIgA does not cause antibody dependent enhancement of infection that could otherwise result in a cytokine storm in the lungs and thus makes targeted mucosal protection, degradation and clearance of foreign bodies safe and unnoticeable by the host organism (See e.g., Iwasaki, A. & Yang, Y., 2020, Nat. Rev. Immunol. 20:339-341).

### SUMMARY OF THE INVENTION

This invention primarily relates to a method to encode for dimeric immunoglobulin A (dIgA1 or dIgA2) and engineered variants (such as dIgA with modified hinge lengths or other modified Fc or constant regions) on a vector as a means to deliver a gene therapy to humans. Specifically, dIgA (dIgA1, dIgA2 or engineered variants) encoded for on a single vector or multiple vectors is the invention. dIgA may be encoded for on a single DNA, or RNA with the use of a furin cleavage site and 2A self-processing peptide between consecutive transgenes. The transgenes that make up dIgA include (1) the immunoglobulin heavy chain, (2) the immunoglobulin light chain and (3) J Chain. These three proteins may be encoded for a on a single genetic element in one or two open reading frames. Where all the transgenes in any one open reading frame will have in the 5′ to 3′ direction a furin cleavage site followed by a 2A self-processing peptide placed between any two consecutive transgenes. The use of a furin cleavage site and 2A self-processing peptide allows for efficient cleavage points between the consecutive transgenes completely removing any trace of the 2A self-processing peptide in the final immunoglobulin. In some cases MZB1 may also be encoded for by the vector. In another embodiment a 2A self-processing peptide is used between consecutive transgenes in a single open reading frame in the absence of a furin cleavage site (see FIGS. 9B and 20B). In an alternative configuration, a 2A self-processing peptide will be used between consecutive transgenes in a single open reading frame in the absence of the furin cleavage site leaving the 2A residue on the C-terminal end of the immunoglobulin protein that is encoded for by the gene that is directly 5′ of the 2A gene on the coding DNA sequence and the mRNA. In other words, excluding the use of the furin cleavage site when using a 2A self-processing peptide will have the effect of incorporating the amino acid sequence of the cleaved 2A peptide on the C-terminal end of the immunoglobulin chain that immediately precedes it and such amino acids will be part of the final immunoglobulin product. In another configuration mRNA encodes for dIgA (dIgA1, dIgA2 or engineered variants) over one or more mRNA vectors (see FIG. 22). When a single mRNA encodes for dIgA it will include a furin cleavage site and 2A self-processing peptide or only a 2A self-processing peptide between any two consecutive transgenes in a single open reading frame.

Potent dimeric immunoglobulins of class A1, A2 or variants specific to (1) a virus (s) (2) a systemic ailment such as allergies (3) fungi (4) bacterial infection (4) cancerous tumor (5) biowarfare agent (6) microbial infection, (7) any ailment (8) an allergen or cause of allergies including IgE and cytokines, (9) a target protein or variant may be discovered or have their V regions or CDR regions derived or discovered from one or more sources exposed to the protein or protein source of interest (any of 1 though 9 and variants but not limited to that list) where such sources of antibodies may include B-cells from humans, mice or other animals with humanized immune systems, transgenic mice, non-human vetebrates (e.g. mouse or rabbit) intended for chimeric antibodies or combinatorial libraries using human B-cells to identify immunoglobulin DNA or using phage display technology to identify potent scFv (single chain variable fragments) or even potent antibody binding fragments (Fab) against the protein or pathogen of interest (e.g. any of 1 through 9 or the pathology associated source) or allergen(s) of interest. In the invention one may take the gene sequence encoding for the safest and most potent immunoglobulins or immunoglobulin V-regions, CDR regions, or even binding regions that can be identified or evolved through mutagenesis, use the gene sequence or a redundant sequence that encodes for the same amino acids of those potent immunoglobulins, CDRs and immunoglobulin V-regions that were discovered in mice or other animals with humanized immune systems, non-human vetebrates (e.g. mouse or rabbit) intended for chimeric antibodies, transgenic mice, or derived from phage display technology and deliver to humans via mRNA, episome or lentivirus for retroviral insertion into host genomic DNA the genetic information to produce the antibodies themselves in a dIgA1 or dIgA2 construct encoding for either monoclonal antibodies or an antibody cocktail of 2 or more immunoglobulins all based on those regions responsible for binding the target (e.g. Complementary determining regions, (CDRs)), V-regions, or the entire polypeptide sequence of the potent immunoglobulins they are based on. In humans, mice, animals with humanized immune systems, animals with engineered immune systems such antibodies may be discovered from CD27+ IgG or IgA memory B-cells or CD+ memory B-cells, plasmablasts B-cells, or B-cells from mice or other animals with humanized immune systems exposed to some or all of the pathogen, pathogenic protein, pathogen associated protein or allergen of interest and affinity matured against it, non-human vetebrates (e.g. mouse or rabbit) intended for chimeric antibodies, transgenic mice or other non-human vertebrate with an engineered immune system or derived from phage display technology such as when using the scFv derived from human B-cells. In one embodiment infusion of mRNA, episomes or retroviral incorporation of DNA via integration competent lentiviral vectors will engender humans with the ability to express dimeric immunoglobulin class A subtypes 1 and also subtype 2 (dIgA1 and dIgA2)—potent for the infectious agent, antigen or allergen of interest—that is modified into secretory immunoglobulin A (SIgA) as part of binding to polymeric immunoglobulin secreting receptor (pIgR) at the basal face of epithelial cells (See FIG. 2) and entering the mucus of organs such as the upper respiratory tract including the lungs, reproductive tract, digestive tract, our urinary tract, our skin exocrine glands of the breasts, all exocrine glands throughout the body. With exception to the heart, nearly, every organ is involved in the processing of a liquid or gas that is transported through a lumen or duct Part of or all of these lumens and ducts are aligned with stratified and pseudo stratified columnar epithelial cells that with polymeric immunoglobulin secreting receptor (pIgR) actively transport dimeric immunoglobulin A (dIgA) from the lamina propria to the mucosa or from the surrounding tissue of the exocrine gland to the exocrine duct where upon entering the mucosa or duct of these organs and glands dIgA is converted into secretory immunoglobulin A (SIgA). Thus, the invention can target any mucosal region or exocrine duct both by using a localized delivery of the gene therapy or intravenous delivery. Additionally, the invention can target beneath the skin. The mucosa of reproductive tract has both SIgA and dIgA because only portions of the epithelial cells of the reproductive tract have polymeric immunoglobulin secreting receptor (pIgR) on the basolateral face. In the invention in cases where the respiratory tract of digestive tract is the mode of allergen or pathogen entry especially the stomach and upper duodenum e.g. to protect against a Helicobacter pylori infection as one example or stomach cancer as another example the gene therapy or protein or mRNA encoding protein based vaccine may optionally be administered through absorption or via endoscopic injection into the lamina propria of the stomach and upper duodenum. In other examples the invention will optionally deliver through endoscopic injection a protein or mRNA based vaccine that encodes for an antigen which may optionally be used with or without the dIgA encoding gene therapy.

When a gene therapy cocktail is used, the infusion of mRNA, episomes or retroviral incorporation of DNA with a lentivirus may also engender humans with the ability to potentially produce any one Immunoglobulin Class G (IgG) subclass 1 (IgG1), subclass 2 (IgG2) and subclass 3 (IgG3), IgA1, IgA2 in addition to dIgA1 and dIgA2. This infusion of mRNA, episomes or retroviral insertion of DNA coding for dIgA1 or dIgA2 engenders humans with two lines of defense against the (1) a virus (s) (2) a systemic ailment such as allergies (3) fungi (4) bacterial infection (4) cancerous tumor (5) biowarfare agent (6) microbial infection, (7) any ailment (8) an allergen or cause of allergies including IgE and cytokines, (9) a target protein or variant.

An additionally benefit of dIgA, which provides SIgA based mucosal immunity is that it is possible to stop foreign bodies and foreign proteins before they breach the epithelial barrier while also avoiding the setting off of destructive inflammatory responses such as allergic reactions or antibody dependent enhancement of infection (ADE). If pathogens are neutralized in the mucus by SIgA1 or SIgA2 or engineered variants it will significantly reduce if not completely eliminate the probability of pathogen or allergen related events that are destructive to human tissue that may be dangerous or life altering to human life. Although, if the pathogen, antigen or allergen did manage to enter the bloodstream by breaching the epithelium a second line of defense dIgA directly beneath the epithelium and a third line of defense dIgA directly beneath the basement membrane would neutralize it and either signal for phagocytosis by neutrophils or macrophages or be transported to the mucosa or duct while dIgA is bound to the target.

### BRIEF DESCRIPTION OF THE DRAWINGS

FIG. 1. depicts a basic schematic of dimeric immunoglobulin class A (dIgA). Additionally depicted is a disulfide where the hinge meets the C_{H}2 domain. That is the disulfide bond is said to be between the final amino acid of the C_{H}2 domains of one heavy chain and its nearest neighboring heavy chain. Further, each constant domain, the hinge or and the V-regions is labeled for one of the four tetramers where each tetramer is a heterodimer. The V-region of the heavy chain is referred to as V_{H}.

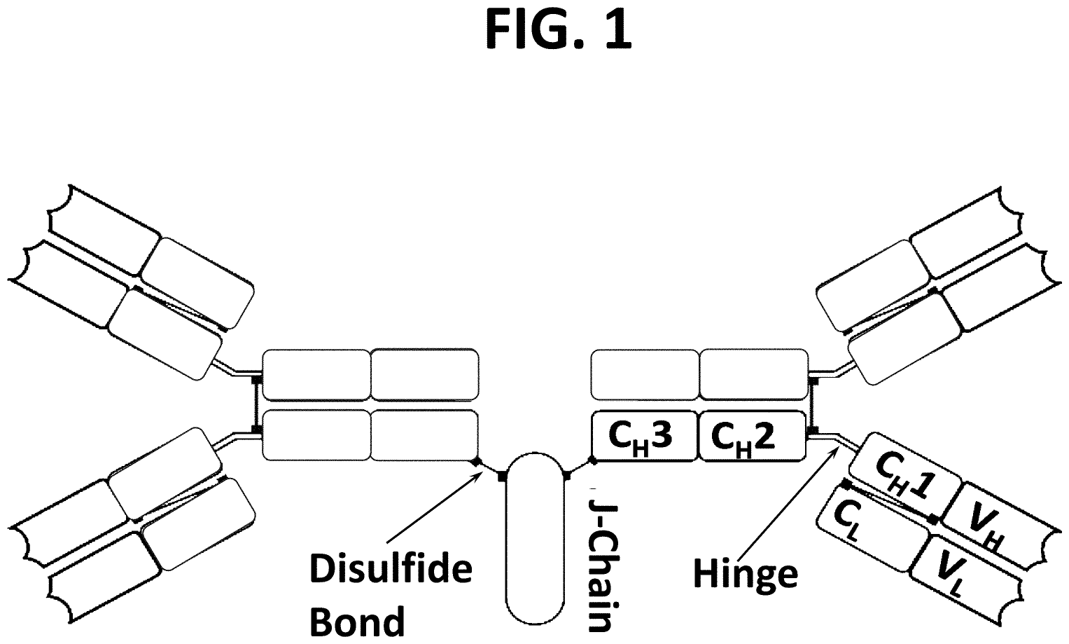

FIG. 2 depicts the mechanism by which Dimeric immunoglobulin classes A1 (dIgA1) and A2 (dIgA2) which includes J chain crosses the epithelium from the basal face—or face that is exposed to the tissue—of the epithelium by first binding to Polymeric Immunoglobulin Secreting Receptor (pIgR) that includes secretory component where it undergoes endocytosis into the epithelium and is transcytosed across the epithelium to the apical face— the face that is exposed to the lumen of the organ of interest or interior of the duct in the exocrine gland that also inclusive of the mouth and nasal cavity and mucus in the lumen of the lungs, lumen of the digestive tract, lumen of the urinary tract, skin, exocrine glands of the breasts, and portions of the reproductive tract—where upon exocytosis to the apical face of the epithelial cells pIgR is cleaved to form secretory component which is released onto dIgA which is then referred to as secretory immunoglobulin class A subclass 1 (SIgA1) or subclass 2 (SIgA2) which refers to the fact that dIgA now has secretory component bound to it. This figure is not drawn to scale nor drawn to convey any structural or molecular information.

FIG. 3 depicts a basic schematic of immunoglobulin class G. For one light chain the Variable Light chain constant region is labeled V_{L} and the constant light chain is labeled C_{L}. The immunoglobulin heavy chain consists of a constant region that is made up of C_{H}3 domain, C_{H}2 domain, the Hinge and C_{H}1 domain. The Fab or antibody-binding fragment is circumscribed and does not include the hinge. Additionally, the Fragment crystallizable (Fc) constant regions is circumscribed to include the hinge.

FIG. 4A depicts SIgA1 and disulfide bonds between J-chain's cysteine 14 and 68 and IgA1 heavy chains penultimate cysteine peptide. Also, shown in the disulfide bond between secretory component cysteine 467 and immunoglobulin class A1 heavy chain cysteine 311. The antibody-binding fragment (Fab) refers to the entire light chain and the Variable Heavy (V_{H}) domain and the Constant Heavy Domain 1 (C_{H1}). The 5 domains D1, D2, D3, D4 and D5 of secretory component are also depicted in this figure. FIG. 4B The structure of SIgA1 is also represented as a crystal structure as a superposition of the 50 best-fit models. The Fab(s) are shown with low resolution as a result of the hinge flexibility.

FIG. 5 Omitted

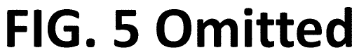

FIG. 6A depicts an Adeno-Associated Virus (AAV) single-stranded DNA vector from left to right or 5′ to 3′ containing a 5′ inverted terminal repeat (5′ ITR), a Promoter which could include an enhancer, the 5′ UTR, a DNA sequence encoding the immunoglobulin heavy chain IgHA1 with no stop codon, DNA encoding a furin cleavage site, DNA encoding a 2A self-cleaving peptide, a DNA sequence encoding the immunoglobulin light chain IgLκ or IgLλ, with stop codon, an internal ribosome entry site (IRES), DNA encoding for the J chain polypeptide necessary for the formation of dimeric Immunoglobulin A (dIgA), the 3′ UTR, a polyadenylation element and the 3′ ITR. In this drawing and most other drawings a box labeled “Kozak then start codon” means that the Kozak consensus sequence from positions −6 to −1 is present (generally “gccacc”) followed by the start codon “atg”. “Kozak then start codon” optionally means “gccaccatg” or it means that the Kozak sequence encoded in the natural or artificial 5′ UTR at its 3′ end and is immediately followed by the “atg” start codon. FIG. 6B depicts an mRNA that may be used to encode for dIgA1. FIG. 6B is similar to FIG. 6A in that the mRNA that would result from FIG. 6A is equivalent to the mRNA represented by FIG. 6B. Although, a modified 5′ cap may be used in place of the natural 5′ cap.

FIG. 7A is similar to FIG. 6A and only differs by the relative location of the DNA encoding for the immunoglobulin heavy chain (IgHA1) IgH and the immunoglobulin light chain IgL(κ or λ). Additionally, FIG. 7A does not depict the 5′ untranslated region (5′ UTR) and the 3′UTR as separate elements. This speaks to the multiple ways to represent the vector constructs that have the same meaning. That is it is understood that there are 5′UTRs and 3′UTRs used in vectors although those sequences but that information is often not included in the vector constructs. FIG. 7B is identical in meaning to FIG. 7A although in FIG. 7B the signal peptides for each of the immunoglobulin chains (Heavy, Light and J) are shown as separate elements. Although, in FIG. 7A and all other figures it is understood that the signal peptides are incorporated into the vector constructs.

FIG. 8 is similar to FIG. 9A but a Woodchuck hepatitis virus post transcriptional regulatory element (WPRE) is not used.

FIG. 9A is similar to FIG. 6A but a furin cleavage site followed by a 2A self-processing peptide are used in place of the IRES. FIG. 9B is similar to FIG. 9A but furin cleavage sites are not used and only a 2A self-processing peptide separates any two consecutive genes.

FIG. 10 is similar to FIG. 11 but a Woodchuck hepatitis virus posttranscriptional regulatory element (WPRE) is not used.

FIG. 11 is similar to FIG. 9A but the positions of J Chain and IgHA1 are swapped.

FIG. 12 is similar to FIG. 14 but a Woodchuck hepatitis virus posttranscriptional regulatory element (WPRE) is not used.

FIG. 13—depicts an Adeno-Associated Virus (AAV) single-stranded DNA vector from left to right or 5′ to 3′ containing a 5′ inverted terminal repeat (5′ ITR), a Promoter which could include an enhancer, the 5′ UTR, a DNA sequence encoding the immunoglobulin heavy chain IgA1 with no stop codon, DNA encoding a furin cleavage site, DNA encoding a 2A self-cleaving peptide, a DNA sequence encoding the immunoglobulin light chain IgLκ or IgLλ with no stop codon, DNA encoding a furin cleavage site, DNA encoding a 2A self-cleaving peptide, DNA encoding for the J chain polypeptide necessary for the formation of dimeric Immunoglobulin A (dIgA) with no stop codon, DNA encoding a furin cleavage site, DNA encoding a 2A self-cleaving peptide, DNA encoding for a MZB1 protein with stop codon, the 3′ UTR, a polyadenylation element and a 3′ ITR.

FIG. 14 is similar to FIG. 12 but the positions of the immunoglobulin light and heavy chains are swapped.

FIG. 15—depicts an Adeno-Associated Virus (AAV) single-stranded DNA vector from left to right or 5′ to 3′ containing a 5′ inverted terminal repeat (5′ ITR), a Promoter which could include an enhancer, the 5′ UTR, DNA encoding for a MZB1 protein with no stop codon, DNA encoding a furin cleavage site, DNA encoding a 2A self-cleaving peptide, DNA encoding for the J chain polypeptide necessary for the formation of dimeric Immunoglobulin A (dIgA) with no stop codon, DNA encoding a furin cleavage site, DNA encoding a 2A self-cleaving peptide, a DNA sequence encoding the immunoglobulin heavy chain IgA1 with no stop codon, DNA encoding a furin cleavage site, DNA encoding a 2A self-cleaving peptide, a DNA sequence encoding the immunoglobulin light chain IgLκ or IgLλ with stop codon, the 3′ UTR, a polyadenylation element and a 3′ ITR.

FIG. 16—is similar to FIG. 15 but the positions of IgHA and IgLκ or IgLλ. are swapped where IgHA has a stop codon and IgLκ or IgLλ. does not have a stop codon.

FIG. 17 depicts a Lentiviral vector from a first or second generation lentiviral delivery system shown at three levels. First in plasmid form with portions cut out as depicted by the double curved lines. Followed by the mRNA form after transcription that is packaged into the lentivirus capsid and further packaged into the lentivirus envelope. Finally depicted last is the DNA form after reverse transcription of the RNA vector that is packaged in a 2nd generation lentivirus delivery system. This vector may be used as a retroviral or viral vector as determined by the functioning of the protein Integrase. FIG. 17 in the 5′ to 3′ direction depicts a 5′ LTR left orientation includes the U3 enhancer and promoter, the R repeat signal and the U5 polyadenylation signal. In this example the vector in the 5′ to 3′ direction depicts the vector to express dimeric immunoglobulin A (dIgA) including the, 5′ LTR, the Promoter that may include an enhancer, including in the vector but the “psi” packaging signal that is located just 3′ of the 5′ LTR, followed by the rev response element (RRE), followed by the central polypurine tract-central termination sequence (cPPT/CTS), the promoter, the 5′ UTR, the immunoglobulin heavy chain for isotype A subclass 1 (IgA1), an internal ribosome entry site (IRES), the immunoglobulin light chain (IgL) that may be kappa or lambda, an IRES, The J chain, an IRES, and Marginal Zone B1 Cell Specific Protein (MZB1), the 3′UTR, a Woodchuck hepatitis virus posttranscriptional regulatory element (WPRE), a polyadenylation element and the 3′ LTR containing the U3, R and U5 elements equivalent to the U3, R and U5 in the 5′ LTR. It should be noted that lentiviral vectors are often depicted in the DNA form following reverse transcriptase of the lentiviral RNA upon release of the capsid.

FIG. 18 is generally similar to FIG. 17 except it depicts a Lentiviral vector from a 3rd Generation self-inactivating lentiviral delivery system. In FIG. 18 the U3 promoter/enhancer on the 5′ end of FIG. 17 is replaced with a CMV promoter to drive transcription as a result of the mutations or deletions of the U3 promoter/enhances (referred to as ΔU3). The ΔU3 on the 3′ end is copied to the 5′ end following reverse transcription.

FIG. 19 depicts the vector after reverse transcription and is generally similar to FIG. 18 except IgHA2 is depicted in place of IgHA1 and a Furin Cleavage Site and 2A self-cleaving peptide is used to express the immunoglobulin heavy and light chains as a single open reading frame.

FIG. 20A is generally similar to FIG. 19 except IgHA1 is depicted in place of IgHA2. FIG. 20B is similar to FIG. 20A but a furin cleavage site is not used in the single open reading frame only a 2A self-processing peptide separates the immunoglobulin heavy and light chains.

FIG. 21 is similar to FIG. 20A except MZB1 is not encoded for by the vector.

FIG. 22 depicts 3 separate mRNA vectors that would be delivered in a single vesicle such as a lipid nanoparticle to the cell of interest to express dIgA1 or dIgA2. As an example such a lipid nanoparticle could be used to deliver the mRNA that codes for a dIgA1 that targets Streptococcus pneumoniae the most common bacteria to cause pneumonia and potentially even other infections that need an immediate remedy. Such targeting for Streptococcus Pneumoniae may take place with highly potent binding affinity for one of the highly conserved Streptococcus Pneumonia epitope.

FIGS. 23A and 23B depicts dIgA1 and SIgA1 binding a lung cancer cell surface receptor. In FIG. 23A dIgA1 is bound to the non-small cell caner malignant tumor that spans the epithelium and basal cells potentially exceeding those boundaries into the lumen. The dIgA1 form is capable being detected by macrophages and neutrophils via the FcαRI to disrupt cancer cell activity and eliminate cancer cells through both opsonization and phagocytosis. At the apical face of the epithelium in the mucosa SIgA1 is bound to the lumen facing face of the same malignant tumor. Additionally, because of the two binding faces of SIgA1 and dIgA1 can prevent metathesis by binding escaping cancer cells on the immunoglobulin face opposite the immunoglobulin face bound to the malignancy. FIG. 23B is similar to FIG. 23A but the example illustrates small cell cancer instead of non-small cell cancer.

FIG. 24 depicts a memory B-cell that has received a Lentiviral vector with the shown vector that is also the vector depicted in FIG. 21. What is shown is that the memory B-cell will initially express as a B-cell receptor (BCR) the naturally encoded immunoglobulin heavy chain and naturally expressing the immunoglobulin light chain. Upon incorporation of the pseudotyped lentivirus integrating the integration competent lentiviral vector through retroviral incorporation into the genomic DNA or alternatively with a integration deficient lentivirus delivery system the cell will express the BCR as the natural IgH and vector encoded IgL as a result of the strong promoter encoded for in the vector that will result in a significantly higher level of expression of the vector encoded IgHA1, IgL and J Chain vs. the naturally encoded IgHG1 and IgL. Upon activation of the BCR by the protein target e.g. by vector encoded IgL detection of the targeted protein the memory B-cell will see one of two differentiation paths. In one path it will differentiated into a memory plasma B-cell that can persist for decades with the support of the right microenvironment secreting immunoglobulins at a rate of 1,000 per second. Alternatively, the memory B-cell can differentiate into a Germinal center B-cell.

FIG. 25 omitted

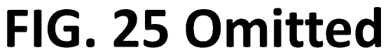

FIG. 26 depicts a vaccination strategy for HIV1, HIV2, Ebola, COVID-19, or any pathology promoting microbe or protein that has a highly conserved protein sequence on the surface that would allow for antibody targeting. This strategy seeks to develop long term and dIgA1 immunoglobulins for decades and is based on the idea that T follicular helper (T_{FH}) cell development will require exposure to antigen. The T_{FH} cell can in turn activate GC B-cells carrying the integrated and episomal delivered lentiviral vector.

FIG. 27 depicts the three lines of defense that is provided with SIgA1 and dIgA1 to prevent type I hypersensitivity. SIgA1 specific to the allergen of interest can bind and agglutinate the allergen in the mucosa of the respiratory tract or in the lungs minimizing the number of allergens that passively cross the epithelium. Additionally, SIgA1 can bind allergens before they have a chance to bind dendritic cell extensions in the mucosa where in cases that dendritic cell (DC) extensions bound allergens and subsequently interact with the T-cell receptor or Tₕ2 Helper cells via DC MHCII presenting allergen oligopeptide fragments that causes the release of IL-4 and IL-13 causing the activation of IgE secreting B-cells increasing the IgE concentration and increasing the incidence of mast cells with two neighboring FcεRIs bound to 2 neighboring IgEs. If allergens passively traverse the epithelium dIgA would bind to allergens and be subsequently engulfed and degraded by eosinophils or macrophages or would be transported to the mucosa while bound to the allergen of sufficiently small size with polymeric immunoglobulin secreting receptor (pIgR).

### DETAILED DESCRIPTION OF THE INVENTION

This present invention arises out of a need to neutralize a variety of systemic pathologies and pathology promoting substances and organisms before they have a chance to cross mucosal tissue or spread far from the mucosal barrier. This invention is a gene therapy focused on expressing immunoglobulins that are naturally produced in large concentrations at mucosal and exocrine tissue that is dIgA1m, dIgA2 and engineered variants that becomes SIgA1, SIgA2 and SIgA engineered variant upon being actively transported from the basolateral face of the epithelium to the mucosa or exocrine duct. The present invention is designed to achieve safety and effectiveness by embracing the immunoglobulins that achieve the therapeutic effect or immunological protection against the foreign body, bacteria, virus, antigen, pathogenic body, pathogenic protein, biowarfare agent or allergen of interest that are developed in humans, developed in mice or other animals with humanized immune systems, developed in transgenic animals, developed in mice and intended for chimeric immunoglobulins, developed from phage display technology where single chain variable fragment (scFv) regions from combinatorial libraries and identified in phage display technology to be therapeutically effective against the pathogen, protein of interest or other antigen, or where immunoglobulins are developed in humans exposed to the foreign body, pathogenic body, pathogen associated protein, antigen or allergen of interest all which may be broadly classified for the purposes of this instant patent application as the target of interest. The invention describes the method to identify the DNA sequence and/or polypeptide sequence of high affinity immunoglobulins expressed in individuals that were exposed to the target of interest. The invention further describes the method to design vectors expressing those immunoglobulins and also dimeric immunoglobulins class A (dIgA1 and dIgA2) necessary for mucosal immunity that encode at a minimum part of the binding regions, V-regions as identified from phage display technology or non-human vetebrates (e.g. mouse or rabbit) intended for chimeric antibodies using some or all of the CDR regions, but more generally at least the Fab regions if not the entire polypeptide sequence of the potent immunoglobulins identified from mice or other animal with humanized immune systems, transgenic mice or the CD27+ IgG memory B-cells, CD27+ IgA memory B-cells from persons that were exposed to or were exposed to and recovered from the foreign body, bacteria, virus, micro-organism, antigen, pathogenic body, pathogenic protein, biowarfare agent or allergen of interest. Delivery systems are also described, which include AAV, adenovirus, lentivirus, lipid nanoparticles and vesicle based delivery systems. While dIgA is privileged to reach the mucosa it can also function effectively in the tissue with increased binding affinities over IgG counterparts. Thus, dIgA1 and dIgA2 may be considered in multiple different regions of the body to provide a therapeutic benefit.

Blood will be collected from any of an individual, a transgenic animal, a mouse with humanized immune system, or a mouse that was infected with, exposed to and ideally has undergone affinity maturation, or lives with any of (1) a virus (s) (2) a systemic ailment such as allergies (3) fungi (4) bacterial infection (4) cancerous tumor (5) biowarfare agent (6) microbial infection, (7) any ailment (8) an allergen or cause of allergies, (9) a target protein or variant. Memory B-cells will be collected from the buffy coat layer (layer that contains white blood cells) of fractionated blood. From humans the Immunoglobulin class A (IgA) CD27+ and class G (IgG) CD27+ memory B-cells or other B-cells with cell surface immunoglobulins will be subsequently isolated. Memory B-cells and plasmablasts are the only B-cells that have both undergone some affinity maturation and bear cell surface immunoglobulins in any appreciable quantity that is required for investigation. Thus, the process of separating cells—that have undergone affinity maturation—based on the affinity of their immunoglobulins for a target of interest can most reliably take place with memory B-cells but is also possible with other B-cells bearing a cell-surface immunoglobulin. There is always the possibility that an IgM B-cell that bears the IgM cell surface immunoglobulin will have high binding affinity and that will be considered as well. Additionally, there is always the possibility that one may isolate a plasma B-cell or memory plasma B-cells that produces immunoglobulins with potent binding affinity for the target of interest. A technique known as B-cell Elispot may be used to identify plasma B-cells or memory plasma B-cells secreting antibodies with potent binding affinity for the target of interest. (See e.g., Crotty, S., Aubert, R. D., Glidewell, J., & Ahmed, R., 2004, J Immunol Methods., 286:111-122.) Generally, B-cell Ellispot may involve using memory B-cells and stimulating them to differentiate into plasma secreting B-cells to detect for secreted antibodies potent for the target of interest. The plasma secreting B-cells are placed in proximity to a plate coated with the antigen of interest (whether self-antigen, foreign antigen or allergen is irrespective of the procedure) where after some time period the cells are washed away and biotinylated detection antibodies are added to the plate to detect for the presence of the secreted antibody bound to the antigen on the plate. Thus, this instant patent contemplates the use of memory B-cells, plasmablast B-cells, memory plasma B-cells and plasma B-cells to identify immunoglobulins, immunoglobulin V-regions with potent binding affinity for the target of interest.

Alternatively, phage display technology that collectively expresses a combinatorial library of scFv with the use of mutagenesis as a means of in vitro affinity maturation of repeated cycles of scFv evolution and bio panning techniques to achieve increasing binding affinities for the target of interest may be used to identify the cDNA encoding for the potent binding affinity. The source of the immunoglobulin cDNA encoding for the scFᵥ may or may not be from persons that were exposed to the target of interest. Another possibility is the use of mice or other animals with humanized immune system or transgenic mice as a means to affinity mature human immunoglobulins in these animals by exposure to the target of interest or part of the V regions may be identified with the use of non-human vetebrates (e.g. mouse or rabbit) intended for chimeric antibodies.

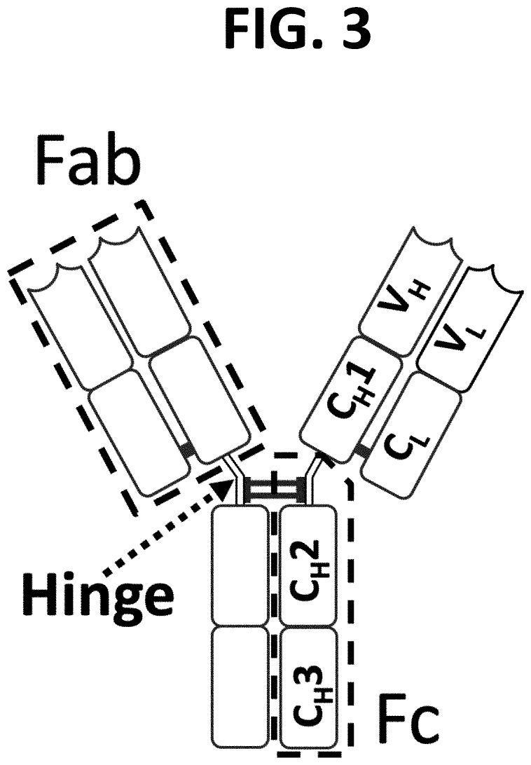

In the invention the genetic information that encodes for that potent binding affinity or the polypeptide sequence that results in that immunity will be determined, evaluated for safety, coupled with constant regions potentially engineered and then incorporated into an mRNA vector, episomal expression vector for expression in muscle cells, B-cells including memory B-cells, Germinal Center B-cells, memory plasma B-cells, a plasma blast, and naïve B-cells), liver cells (such as hepatocytes), splenocytes, and other potential cells or retroviral incorporation into genomic DNA via a lentivirus. In the invention those episomes would be delivered as via an adeno-associated-virus (AAV) vehicle as single-stranded DNA that would be converted into double-stranded DNA by the host genetic machinery, adenovirus vehicle, mRNA, lentivirus as RNA that would be reverse transcribed into double-stranded DNA in the host by reverse transcriptase contained in the lentivirus capsid. In the invention if human donors or mice or other animals with humanized immune systems, transgenic mice or non-human vetebrates (e.g. mouse or rabbit) intended for chimeric antibodies are used as the source of potent immunoglobulins or potent binding affinity episomes that encodes for at a minimum immunoglobulins with V regions if not the Fab (See FIG. 3) or even F(ab′)2 that exactly match those V Regions if not the entire polypeptide sequence of potent immunoglobulins specific to the target of interest expressed in the mice or other animal with humanized immune system, transgenic mice, or humans that were exposed to the target of interest. Additionally non-human vetebrates (e.g. mouse or rabbit) intended for chimeric antibodies may have some or all of the CDR regions incorporated into a human immunoglobulin construct. In the invention if phage display technology is used the recombination of the identified V_{H} and V_{L} regions will be evaluated against heavy chain constant regions of different isotypes and subclasses and the two different light chain constant regions IgL(κ or λ) respectively, where the immunoglobulin constant regions may be engineered to modulate effector functions. If an antibody cocktail of immunoglobulins is used it could include both immunoglobulin class G (IgG), immunoglobulin class A (IgA) and dIgA, which is converted to secretory IgA (SIgA) as part of being transported across the epithelium into the lumen of the upper respiratory tract, lungs, gastrointestinal tract, urinary tract, skin, endocrine glands and reproductive tract is the primary immunoglobulin responsible for mucosal immunity will also be encoded for in the antibody cocktail. The mucosa of reproductive tract has both SIgA and dIgA because only portions of the epithelial cells of the reproductive tract have polymeric immunoglobulin secreting receptor (pIgR) on the basolateral face. Thus, it is through the specific method of gene therapy (e.g. mRNA, DNA, retroviral insertion) that collectively code for a single dIgA antibody or an antibody cocktail mixture that mucosal protection from the target of interest will be achieved. The immunoglobulins of interest will produce full or even engineered immunoglobulins to modulate effector functions and could be modified to increase half-life. (See e.g., Iwasaki, A. & Yang, Y., 2020, Nat. Rev. Immunol. 20:339-341.)

The various compositions and methods of the invention are described below. Although particular compositions and methods are exemplified herein, it is understood that any of a number of alternative compositions and methods are applicable and suitable for use in practicing the invention. It is understood that vector constructs may include additional DNA, such as a 5′ Untranslated Region (5′ UTR) and 3′ UTR between or preceding cis-acting signals and transgenes as well as spacer DNA between or before cis-acting signals and viral signals such as LTRs, ITRs, psi, cPPT/CTS, etc. It is also understood that evaluation of the immunoglobulin expression constructs for the vectors specified in this invention can be implemented using methods standard in the art. New vector constructs are also proposed to enable the expression of dimeric immunoglobulin A (dIgA), which becomes secretory immunoglobulin A (SIgA) as part of crossing an epithelial cell from the basal to apical face. Additionally, it is understood that there may be a number of methods and assay modifications that can be used to isolated memory B-cells based on relative affinity for antigens of interest by a flow cytometry or Fluorescence activated cell-sorting (FACS) technique. This should not attenuate the value of any particular cited method. Additionally, the value is further justified by the incorporation of genetic information to expression all or part of those high affinity immunoglobulins Fab, V-region, scFᵥ or partial binding element from non-human vetebrates (e.g. mouse or rabbit) intended for chimeric antibodies, in the context of complete immunoglobulin a mRNA, non-viral, viral or retroviral vector construct.

The methods of this invention will utilize unless specified otherwise modern techniques of cell molecular biology, chemical biology, microbiology, biochemistry and immunology. Many of such techniques are explained substantially in the publication literature and are well understood to those skilled in the art.

### 1. Definitions

Unless stated otherwise all terms used herein have the same meaning that they would to one skilled in the science, art and practice that is utilized in the present invention which include terms from methods in molecular biology, immunology, microbiology, chemical biology, recombinant DNA technology, biochemistry and virology. These terms are well within the knowledge of those with in depth knowledge or skill of the art.

The term “conserved polypeptide sequence” as used herein refers to polypeptide sequences that have remained evolutionally conserved possibly due to functional constraints. This bears relevance in the innate immune system, which relies on pattern recognition of conserved polypeptide sequences to recognize antigens of pathogens.

The term “affinity maturation” as used herein refers to the maturation of an immature B-cell through both isotype switching and somatic hyper mutation and a memory B-cell through somatic hyper mutation that occurs through CD4+ helper T-cell activation of B-cells that have engulfed and degraded an antigen.

The term “potent” as used herein refers to binding affinity of immunoglobulins. Potent is meant to refer to a binding affinity of the immunoglobulin for the foreign antigen produced by the pathogen where the binding affinity is sufficiently high to warrant further investigation for the purposes of expressing the immunoglobulin or a derivative of that immunoglobulin as intended for episomal expression of immunoglobulins.

The term “coding DNA” or “cDNA” as used herein refers to DNA that encodes for the polypeptide of interest. cDNA is effectively mRNA but in mRNA the thymine in cDNA is replaced with uracil in mRNA. However, for the purposes of this instant patent the cDNA is more concerned with the polypeptide sequence that it codes for. In that sense the degeneracy of the genetic codes considers any codon that codes for the amino acid to be an acceptable replacement for a cDNA codon. This is because the concern is the polypeptide sequence where the cDNA that codes for it does not matter due to the degeneracy of the genetic code. However, it is recognized that the efficiency with which an amino acid can be polymerized onto the protein being synthesized by the ribosome is dependent on the codon specifically even though three different codons may code for the same amino acid they may be polymerized onto the developing protein at different rates in a codon dependent manner.

The term “dissociation constant” or (K_{d}) as used herein refers to that immunoglobulin relative proportion or ratio of unbound immunoglobulin to the antigen of interest vs. immunoglobulin bound to the antigen of interest at any instantaneous moment or as a portion of time or ratio that an immunoglobulin is unbound to its antigen vs. bound. K_{d} is often described as (K_{off}/Kₒₙ).

The term “delivery system” as used refers to any system that contains and delivers molecules to the host cell.

The term “donor” as used refers to a human source of biological material and thus anything derived from the donor. Specifically, the terms donor is used to refer to immunoglobulin polypeptide sequences or the coding DNA (cDNA) that encodes for them that is derived from a single cell from the blood of a person that is infected with or has been exposed to or exposed to and recovered from the virus, pathogen, bacteria, antigen, pathogenic protein, systemic pathogenic ailment, biowarfare agent or allergen of interest. However, the cDNA is not required because of the degeneracy of the genetic code and thus, the polypeptide sequence matters most.

What is meant by a donor based immunoglobulin polypeptide sequence or domain is that the exact sequence is derived from the cell of the donor or human that provided blood and refers to an immunoglobulin or immunoglobulin element with therapeutic binding affinity for its target as per the context.

The term “episome” as used herein refers to DNA that resides in the nucleus of the host cell as an extra-chromosomal DNA and does not integrate into the genomic DNA of the host. The episome is said to be a separate artificial chromosome. Although, an episome can be made to be non-replicating or could be designed to be replicating. When an episome is non-replicating when the cell divides into 2 daughter cells only one of the two daughter cells will contain the episome. The concentration of episomes in the host animal for example will reduce with time that is a function of the lifespan of the different cells where the episomes resides.

The term “therapeutic binding affinity” as used herein refers to the antibody binding to any protein of interest including variants of that protein when specified, as well as other body of interest such as a molecule or macromolecule where the desired therapeutic effect is achieved with that binding affinity in the immunoglobulin of interest. That is the binding affinity that most effectively prevents pathology associated with the antibody's target such as a virus, pathogen, bacteria, microorganism, systemic pathological ailment, cancer, antigen, biowarfare agent or allergen.

The term “J chain” also referred to as “Joining Chain” as used herein refers to a 159 amino acid polypeptide that includes a signaling peptide that is cleaved to a 137 amino acid polypeptide that forms a cysteine bond to (A) 2 immunoglobulin class A subclass 1 through a cysteine bond between the penultimate cysteine on each immunoglobulin heavy chain to each of the C14 and C68 cysteines of J chain. (B) 2 immunoglobulin class A subclass 2 through a cysteine bond between the penultimate cysteine on each immunoglobulin heavy chain to each of the C14 and C68 cysteines of J chain. The resulting immunoglobulin is a tetramer of immunoglobulin heterodimers where each heterodimer is made up of 2 immunoglobulin heavy chains and 2 immunoglobulin light chains that includes J chain as described in this paragraph.

The term “immunogenicity” as used herein refers to the ability of a foreign substance, such as an antigen, molecule, macromolecule or allergen to provoke an immune response in the body of a human and that may result in side effects and health complications.

The term “isotype switching” as used herein refers the changing of an immature IgD or IgM B-cell to an IgG, IgA or IgE B-cell as part of affinity maturation. Isotype switching exclusive of somatic hyper mutation may enhance the specificity of an immunoglobulin for its antigen.

The term “immunocompetent” as used herein refers to individuals that have the ability to produce and further develop an immune response following exposure to an antigen.

The term “pseudotyped” as used herein is the process of producing viruses that is viral or retroviral vectors in combination with foreign viral envelope proteins. A virus that is said to be pseudotyped is also referred to as a pseudovirus.

The term “daughter” cell as used herein refers to any of the cells formed when the cell undergoes cell division by mitosis.

The term “effector functions” refers to the signaling activity of antibodies such as the immunoglobulins. The immunoglobulin heavy chain constant regions specifically the Fc and the pFc′ constant domains are inclusive of effector signaling regions of the immunoglobulin. Different immunoglobulin isotypes and subclasses have different effector functions via different receptors for their Fc regions. Effector functions of immunoglobulins communicate with other cells or soluble proteins in the immune system through receptor mediated processes and signal to them to carry out a function or relay a signal to another type of cell. Effector functions include neutralization, opsonization, sensitization for killing by natural killer cells, activation of the complement system, activation of macrophages or phagocytes and activation of mast cells and basophils, antibody-dependent cellular cytotoxicity and antibody-dependent cellular phagocytosis.

The term “small pre-B-cell” refers to a state in B-cell development while the destined B-cell is in the bone marrow. V to J gene rearrangements of the immunoglobulin light chain on chromosome 2 (Kappa locus) or chromosome 22 (Lamba locus) takes place.

The terms “genomic DNA” refers to chromosomal DNA specific to the organism and thus is expected to be found in most cells of the organism. Genomic DNA represents the bulk of the genetic material of the organism. Other genetic material may be mitochondrial DNA and gamete DNA.

The term “non-viral vector” may refer either to a virus or viral particle containing DNA capable of functioning as an episome in the nucleus but is not contained within a capsid. Non-viral vectors and transfer plasmids contain structural and/or functional genetic elements that are primarily derived from a virus but not part of a viral delivery system.

The term “self-antigen” may refer to a protein, polypeptide of macromolecule that is produced by the organism. Self-antigens are to be distinguished from “antigens” that are proteins, polypeptides or macromolecules produced by an external organism or “viral antigens” that are produced by a virus.

The term “systemic pathologies” may refer to a health disorder whose basis of pathology is due to internal factors related to and encoded for in protein levels, mutations, DNA, RNA or cancer. This is to be distinguished from an “external pathogen” that is a pathogenic protein, virus, fungi or other microorganism that is capable to causing pathology.

The term “pathology” may refer to the specific damage sustained by the host organism as a result of any cause. Pathology is broadly defined and may include tissue damage, tissue compromise, cell loss, cancer, allergic reactions, cell malfunction, organ malfunction, organ malformation, tumors, imbalances in cell levels or imbalances in metabolic levels, as well as the psychological consequences of any imbalance.

The term “retroviral vector” refers to a viral vector or plasmid containing structural and functional genetic elements, or portions thereof, that are primarily derived from a retrovirus that is intended for retroviral incorporation into DNA.

The term “lentiviral vector” refers to a vector or plasmid containing structural and functional genetic elements, or portions thereof, including long terminal repeats (LTRs), psi encapsidation signal, central polypurine tract/central termination sequence (cPPT/CTS), rev response elements (RRE) that are primarily derived from a lentivirus.

The terms “integration-deficient lentiviral vector,” may be used to refer to lentiviral viral vectors that are capable of functioning in the host cell as an episome and cannot integrate into the host genomic DNA or is highly unlikely to integrate into host genomic DNA.

The term “somatic hypermutation” may be used to refer to a process that can occur in B-cells. Where Germinal Center memory B-cell V-regions undergo point mutations to produce variant immunoglobulins some of which may have higher affinity for the antigen that was the cause of the somatic hypermutation process. Somatic hypermutation occurs during many stages of B-cell development and during affinity maturation when germinal center B-cells enter the GC dark zone and undergo proliferation. When memory germinal center B-cells become activated by CD4+ helper T-cells they can assume one of three states of differentiation becoming a memory B-cell, memory plasma secreting B-cell or may re-enter the dark zone and undergo further proliferation where each daughter cell will have undergone different mutations in the V-regions as part of the process of affinity maturation. (See, e.g. Janeway, C., Travers, P., Walport, M., and Shlomchik, M., Janeway's Immunobiology, Ninth Edition. 2016. Garland Science: New York, N.Y. ISBN: (Paperback) 978-0815345053)

### 2. Isolation, Identification and Characterization of a Human and Non-Human Vertebrate Source of Immunoglobulins or Human Immunoglobulins with High Affinity or Therapeutic Binding Affinity for the Antigen or Pathogen Associated Protein, Biowarfare Agent, Bacteria, Fungi or Allergen, Immune System Protein, or Self-Antigen of Interest

The peak affinity of one's immunoglobulins for key antigens or protein targets such as highly conserved cell surface antigens in viruses, bacteria, fungi, microorganisms and other pathogens after affinity maturation has taken place is an important factor in one's ability to fight pathogens that evade the innate immune system. The innate immune system relies in part on evolutionarily conserved polypeptide sequences to detect pathogens. When the peptide sequences of proteins produced and especially presented on the surface of viruses lack a minimal degree of sequence similarity necessary to detect such evolutionarily conserved peptide sequences the participation of the adaptive immune system is required to directly detect such specific pathogens. With an increased number of iterations of exposure to pathogenic proteins and antigens the adaptive immune system can increase its affinity for antigens through a process of affinity maturation. When a B-cell becomes immature it typically starts out as an immunoglobulin class M (IgM) or immunoglobulin class D (IgD) B-cell or expressing both that also bears a cell surface immunoglobulin class M receptor or IgD receptor or both IgD and IgM B-cell receptors (BCRs). Part of the B-cell development process consists of going from the early pro-B-cell to small pre-B-cell stage where immunoglobulin gene element rearrangements occur and are evaluated to ensure productive matches and also the undeveloped B-cell detects in the bone marrow for self-antigens. In the Bone marrow when the affinity for a self-antigen is above a specific threshold the undeveloped B-cell can be rescued by gene rearrangements of gene elements of the V-region and is known as “receptor editing”. If repeated receptor-editing fails to produce an immunoglobulin chain that are not strongly reactive to self-antigens the undeveloped B-cell will undergo apoptosis. Immature B-cells have heightened sensitivity to self-antigens and can continue to undergo receptor editing or antigen-induced apoptosis. (See e.g., Melamed, D., 1998, Cell. 92:173-182) However, about 20% of immature B-cells have some reactivity to self-antigens (See e.g., Wardemann, H., et. al., 2003, Science 301:1374-1377). As without this we would not be able to sufficiently generate an adaptive immune response to a broad range of antigens. Immature B-cells undergo further differentiation into mature naïve B-cells that continue to bear cell surface immunoglobulins of IgM or IgD.

In phase 1 of the primary immune response mature naïve B-cells leave circulation and enter secondary lymph nodes and can engaged in antigen encounter with follicular dendritic cells. B-cells then process antigen and present it on their MHC class II to the T-cell receptor (TCR) of T-helper cells. Upon activation by T-helper cells at the T-cell/B-cell border the B-cell undergoes proliferation in the follicle with three possible fates: a Germinal Center independent memory B-cell bearing an IgM receptor, short lived plasma secreting IgM B-cell with a half-life of a few days or a Germinal Center B-cell that will enter the dark zone and undergo proliferation and somatic hypermutation. A secondary encounter with antigen from a follicular dendritic cell followed by processing presentation on the MHC class II to the TCR of T-follicular-helper cells results in activation of the germinal center B-cell that can undergo isotype switching to IgG(1, 2, 3 or 4) or IgA(1 or 2) and can be dependent on the lymph node. For example, lymph nodes that support the mucosa of any organ tend to cause IgM B-cells to class switch to IgA. They can become a memory B-cell baring the cell surface immunoglobulin, a memory plasma B-cell that can persist for decades depending on the support from the local environment which is often the bone marrow, a plasma blast that is in the process of converting to a memory plasma secreting B-cells or can reenter the dark zone for further proliferation and somatic hypermutation repeating the cycle. IgG and IgA Memory B-cells upon activation can differentiate into a long-lived memory plasma B-cell or can reenter the dark zone for further proliferation and somatic hypermutation. T follicular helper cell (T_{FH} cell) Germinal Center B-cell interactions are important for the generation of germinal center B-cells. (See, e.g. Akkaya, M., Kwak, K. & Pierce, S. K., 2020, Nat Rev Immunol., 20:229-238). The long lived plasma B-cells may migrate to the bone marrow where they can be sustained for decades as a result of the microenvironment in the bone marrow and even potentially the stomach and small intestine. Although, the memory plasma B-cells in the small intestine and stomach may be derived directly from the local lymphnodes supporting the organ or may migrate to the interstitium of the stomach and small intestine while not being supplied from local lymphnodes. (See, e.g. Akkaya, M., Pierce, S. K., 2020, Nat Rev Immunol., 20:229-238; Also see, Landsverk, 0. J., Jahnsen, F. L, et al., 2017, J. Exp. Med. 214:309-317; Khodadadi, L., Hiepe, F., 2019, Frontiers in immunology, 10:721)

The repeated cycle of re-entry into the dark zone allows the affinity maturation process where cell surface immunoglobulin bearing B-cells undergo a repeated cycle of proliferation and somatic hypermutation potentially increasing the affinity for the antigen of interest. Both these processes can potentially result in B-cells with reactivity to self-antigens. Naïve B-cells give rise to both lymphoblasts that secrete antibodies and memory B-cells upon activation from a pathogenic antigen and the activation of CD4+ helper T-cells. Through, repeated cycles of proliferation and somatic hypermutation of germinal center memory B-cells the resulting memory B-cells and the resulting daughter long-lived plasma B-cells can develop a greater affinity for the antigen of interest. (See, e.g. Akkaya, M., Pierce, S. K., 2020, Nat Rev Immunol., 20:229-238; Also See e.g., Ziegner, et. al., 1994, Eur. J. Immunol. 24:2393-2400; Shlomchik, M. J., et. al., 1990, Prog. in Immunol. Proc. 7:415-423; Berek, C., & Milstein, C., 1987, Immunol Rev. 96:23-41; Ochiai, K., Sciammas, R., 2013. Immunity, 38:918-929.). It is through this process that affinity-matured immunoglobulins can be produced by B-cells and memory B-cells can for the most part generally be considered to express immunoglobulins that have high affinity for an antigen of interest. Additionally, during the course of any somatic mutation process that results in memory B-cells the possibility exists that memory B-cells can have a higher affinity for self-antigens. Although, these memory B-cells are unlikely to be activated by CD4+ helper T-cells. There is a panel of assays and additional assays that are used to determine if immunoglobulins have reactivity to self-antigens. Although, it is not comprehensive because of the vast number of proteins produced in the human body. Thus, perhaps the best assay would be based on human bone marrow. (See e.g., Tiller, T., et. al. 2007, Immunity, 26:205-213; Also see e.g., Fsuiji, M., et. al. 2006, The Journal of Experimental Medicine 203:393-400, 2006; Wardemann, H., et. al., 2003, Science 30:1374-1377).

Vectors may be created that encode for those immunoglobulins to be evaluated. Other biological sources such as a transgenic animal, a mouse with humanized immune system, or a mouse that was infected with, exposed to, has immune specificity to, or lives with any of (1) a virus (s) (2) a systemic ailment such as allergies (3) fungi (4) bacterial infection (4) cancerous tumor (5) biowarfare agent (6) microbial infection, (7) any ailment (8) an allergen or cause of allergies, (9) a target protein or variant (10) an immune system protein such as IgE or a cytokine. may be used to identify potent immunoglobulins for the target of interest. Additionally, in some embodiments the CDR regions and in other embodiments the V-region polypeptide encoding nucleic acids of the heavy (IgH) and light chain (IgL) of the immunoglobulins may be incorporated into a vector construct that could use either the constant region DNA identified in the B-cell expressing the identified immunoglobulin of interest or another source of genetic information may replace all or part of the constant region DNA that may also include isotype switching of constant region genetic information, mixes of two constant regions from two isotypes that may be accomplished with combinations of Fab (from donor) and natural or engineered Fc domains or F(ab′)2 (from donor) and natural or engineered pFc′ domains. Additionally, the immunoglobulin hinge length may be modified. Central towards this end is the production of dIgA1 and dIIgA2 to enable mucosal immunity against the (1) virus (s) (2) systemic ailment (3) fungi (4) bacterial infection (4) cancerous tumor (5) biowarfare agent (6) microbial infection, (7) any ailment (8) an allergen or cause of allergies such as IgE or cytokines, (9) target protein or variant of interest.

A human (ideally between 21 and 55 years old), a transgenic animal, a mouse or other animal with humanized immune system, or a mouse that was infected with, exposed to, has immune specificity to or lives with any of (1) a virus (s) (2) a systemic ailment such as allergies (3) fungi (4) bacterial infection (4) cancerous tumor (5) biowarfare agent (6) microbial infection, (7) any ailment (8) an allergen (9) a target protein or variant (10) in the case of non-human vertebrates a human immune system protein such as Immunoglobulin class E (IgE) or a cytokine. has their blood drawn. The blood sample will be allowed to separate into its three layers or can be subjected to blood fractionation method such as density gradient centrifugation. The buffy coat layer, which contains the peripheral blood mononuclear cells (PBMCs) that includes memory B-cells is collected.

One method but certainly not only method that could be used to isolate Human CD27+ IgG+ memory B-cells would be with EasySep™ Human IgG+ Memory B-Cell Isolation Kit′.

This kit can be purchased from STEMCELL Technologies in Cambridge, Mass. This kit also includes an option to purchase an additional separation assay to separate out CD27+ IgA+ memory B-cells. In a similar sense all of the memory B-cells could also be isolated and analyzed together. Although, it is preferred to analyze the CD27+ IgG+ and CD27+ IgA memory B-cells with option to analyze separately or together. The separation of different memory B-cell isotypes is achieved through the use of immunoglobulins specific for the heavy chain Fragment crystallizable (Fc) constant regions of a specific memory B-cell isotype. Using the STEMCELL Technologies method primary human CD27+ memory B-cells can be isolated by immunomagnetic bead isolation followed by a depletion cocktail to deplete Human IgM/IgD/IgA B-cell Depletion Cocktail leaving the CD27+ IgG memory B-cells as a pure sample. Also available is a solution to deplete Human IgA B-cells. Thus, both IgG and IgA memory B-cells will be collected. (Catalog #17868, STEMCELL; Website: https://www stemcell.com/easysep-human-igg-memory-b-cell-isolation-kit.html#section-data-and-publications) Patient PBMCs may be stored frozen and thawed before use. (For an alternative method to isolate CD27+B-cells see e.g., Tiller, T., et. al. 2007, Immunity, 26:205-213) Other methods to separate CD+ memory B-cells may be used where such B-cells can be subsequently separated with FACS or magnetic isolation experiments that take advantage of differences in the immunoglobulin constant regions on the cell surface immunoglobulins as a means to separate out IgG from IgA B-cells or plasmablasts. ¹ Website: https://www stemcell.com/easysep-human-igg-memory-b-cell-isolation-kit.html#section-data-and-publications

It is difficult to perfectly separate the IgG memory B-cells by their subclasses e.g. IgG1, IgG2, IgG3 etc. IgG1+ memory B-cells generally make up 60%-70% of IgG memory B-cells in healthy donor serum. IgG2+ memory B-cells make up about 25% of IgG memory B-cells and IgG3+ memory B-cells make up about 9% of IgG memory B-cells. This difficulty in separation suggests that the all IgG subclass have heavy chain constant regions that are topographically similar. (See e.g., Tiller, T., et. al. 2007, Immunity, 26:205-213) However, this is not important because downstream steps will address these differences accordingly.

It is understood that there are established methods to isolate memory B-cells from transgenic mice or rabbit, mouse with humanized immune system and from mice that are well known to those familiar with the art. IgG memory B-Cells or IgA memory B-cells will then be separated by relative binding affinity through a flow cytometry technique such as fluorescence activated cell sorting (FACS). The flow cytometry analysis of the relative binding affinities of memory B-cells will be dependent on labeling proteins such as antigens with a fluorescent tag to allow detection of antigen bound B-cells by the FACS that will employ the use of a competing binding protein such as the protein that the target protein binds. (E.g. if the target protein binds the CCR5 receptor, then it could be used as a competing protein in the assay e.g. in a soluble form) There are an extensive number of well-established methods to determine the relative binding affinities of cell surface receptors for the antigen of interest that are well known to those in the art. As an example, one may use in competition with memory B-cells to bind to the CCR5 receptor-binding domain (RBD) of the HIV glycoprotein. Assays may be designed to determine if memory B-cells binding affinities greater than that of CCR5 for the HIV glycoprotein. There are well-established methods for approximate quantification of binding affinity of immunoglobulins using methodology, known to those of skill in the art. (See e.g., Weiss, G. E., et. al., 2012, Journal of immunological methods, 375:68-74.; Hunter, S. A., & Cochran, J. R., 2016, Methods Enzymol. 580:21-44.; Li, P., Selvaraj, P., & Zhu, C., 1999, Biophysical journal, 77:3394-3406.; Amanna, I. J., & Slifka, M. K., 2006, Journal of immunological methods, 317:175-185)

Immunoglobulin DNA sequences of the CD27+ IgG+ and CD27+ IgA+ memory B-cells, CD+ memory B-cells or plasmablasts can be obtained through established methods. (Niu, X., et al.. 2019, Emerging microbes & infections, 8:749-759; Cao, Y., 2020, Cell, 182:73-84) These are well-established methods known to those of skill in the art and also available from a variety of commercial sources with standardized protocols. Further, such a protocol to obtain the DNA encoding for potent immunoglobulins was previously established to allow for efficient isolation and identification of neutralizing monoclonal antibodies (mAbs) in different infectious diseases. (See e.g., Setliff, I., et. al., 2019, Cell, 179:1636-1646; Wu, X., et. al., 2010, Science, 3295:856-861) Alternatively, memory B-cells specific to the antigen can be subjected to an antigen isolation assay where antigen biotinylated to magnetic beads is competed for between memory B-cell surface immunoglobulins such as IgG+ or IgA+ of different memory B-cells. Magnetic pull downs will only capture those memory B-cells with the greatest relative binding affinity for the antigen of interest. B-cells can also compete against a protein of interest that is present in the solution with known binding affinity to the biotinylated antigen. (See e.g., Cao, Y., 2020, Cell, 182:73-84; Niu, X., et al., 2019, Emerging microbes & infections, 8:749-759) These are established methods known to those of skill in the art and also available from a variety of commercial sources with standardized protocols. However, for any particular antigen, assays may have to be developed using established methods known to those of skill in the art. If magnetic beads are used what would result are a cohort of memory B-cells that could then be separated with a subsequent competitive binding flow cytometry assay such as FACS as described herein. Alternatively, the memory B-cells isolated from the magnetic pull down separation can be assessed for binding affinity. Those memory B-cells with among the more desirable affinities may be identified from one or more of (A) the magnetic pull down isolations assay followed by FACS separation (B) magnetic pull down isolations (C) FACS assays. (Niu, X., et al.. 2019, Emerging microbes & infections, 8:749-759)

The K_{d} threshold for determining which memory B-cells affinity memory B-cells and achieving a mix of IgG1, IgG2, IgG3, IgA1 and IgA2 memory B-cells each that meet a K_{d} criteria. Preferentially, the K_{d} criteria for memory B-cell selection would be in the single digit picomolar—if not femtomolar—range. Such dissociation constants can be measured by surface plasmon resonance for the individual immunoglobulin. Although, it is understood that converting an IgG immunoglobulin into an IgA1 recombinant isotype can result in a 1 to 2 order of magnitude increase in binding affinity. Additionally a neutralization titer (NT50) inhibitory dose may be determined to assess the neutralization potential of immunoglobulins of known concentration in solution for an antigen of interest. The NT50 is defined as neutralization titers [50% inhibitory dose (ID50) or the 50% inhibitory concentration (IC50)] that is defined as the reciprocal of the serologic reagent dilution (or concentration for purified reagents) that caused a 50% reduction in relative luminescence (RLU) compared to virus control wells after subtraction of background RLUs. (See e.g., Robbiani, D. F., et al., 2020, Nature, 584:437-442; Antonio, E., et. al., 2020 bioRxiv, 2020.05.21; Sarzotti-Kelsoe, M., et. al., 2014, Journal of immunological methods, 409:147-160).

The DNA sequences that codes for the immunoglobulins expressed by the memory B-cells may be determined by one of several established methods. Such techniques are well established and understood by those that practice the art. Antigen-binding memory B-cells or plasmablasts may be identified after a magnetic pull down isolation experiment and/or flow cytometry. Identification of the immunoglobulin heavy and light chain polypeptide sequences may be used to incorporate such genetic information into a plasmid transfected into e.g. a HEK-293 cell or human hepatocytes for expression and evaluation of the secreted antibody. (see e.g., Setliff, I., et. al., 2019, Cell, 179:1636-1646; Cao, Y., 2020, Cell, 182:73-84;)

Discussed throughout this instant patent application the possibility exists that memory B-cells can have a high affinity for self. There is a panel of assays and additional assays that are used to determine if immunoglobulins have reactivity to self-antigens. There are well-established methods to carry out these panel assays with monoclonal antibodies known to those of skill in the art. (See e.g., Tiller, T., et. al. 2007, Immunity, 26:205-213; Also see e.g., Fsuiji, M., et. al. 2006, The Journal of Experimental Medicine 203:393-400, 2006.; Wardemann, H., et. al., 2003, Science 30:1374-1377). Another described global self-antigen assays can also be used to evaluate immunoglobulin for self-reactivity. (See e.g., Vale, et. al., 2016, Front. Immunol. 7; Nobrega, A., et. al., 1993, Eur J Immunol., 23:2851-2859; Haury, M., et. al., 1997, Scand. J. Immunol. 39:79-87) (See e.g., Weiss, G. E., et. al., 2012, Journal of immunological methods, 375:68-74)

### 3. Modification or Incorporation of Constant Regions of Natural or Engineered Sources of Human Derived or Humanized Mice, Transgenic Mice, Non-Human Vetebrates (e.g. Mouse or Rabbit) Intended for Chimeric Antibodies, Derived Human Immunoglobulins or V_{H} and V_{L} Regions Identified from Phage Display Technology with High Affinity for the Viral Antigens of Interest

Those immunoglobulins discovered from human B-cells, mice, transgenic mice or mice with humanized immune systems that are deemed to have “therapeutic binding affinity” generally high affinity for antigen of interest while also being at sufficiently low reactivity for self-antigens will have their DNA incorporated into an expression vector for further evaluation. Additionally, high affinity V_{H} and V_{L} regions identified from phage display technology will be incorporated into full length immunoglobulin heavy and light chains for further evaluation. That evaluation of immunoglobulins identified from any of the sources may further include modifying the constant regions of the immunoglobulins using a variety of different sources of human genetic information or through engineering the constant regions to improve effector functions. This approach may include using constant regions of different isotypes or different subclasses and engineering of the constant region that are detected by Fc receptor on macrophages and monocytes as an example to reduce antibody-dependent enhancement of infection. All such immunoglobulins may be evaluated in the battery of tests that include self-reactivity assays and binding assays as described in this document.

Plasmids will be created that encode for immunoglobulins to be evaluated. The V-region and some of the constant region DNA of the heavy (IgH) and light chain (IgL) of the immunoglobulins will be incorporated into a vector construct that could use either the constant region DNA of another isotype and/or subclass or another source of human genetic information may replace all or part of the constant region DNA for the immunoglobulin heavy and/or light chain that may also include (A) class or subclass switching of constant region genetic information (e.g. replacing all of the IgG2 constant region with IgG3 or IgA1), (B) mixes of two constant regions from two isotypes or subclasses (e.g. replacing part of the IgG2 constant region with an IgA1 or IgA2 constant region; although generally at a minimum the Fc or pFc′ region would be derived from IgA1 or IgA2 since the C_{H}3 (Constant Heavy 3) region is responsible for complexing with J Chain (C) replacing the Fc part of the constant region coding DNA (polypeptide sequence) of a particular subclass with the constant region DNA of the subclass from a another source of genetic information with the emphasis of low immunogenicity.

In consideration of human derived immunoglobulins or immunoglobulins derived from mice with humanized or engineered immune systems likely, the immunoglobulin light chain (IgL) constant region would be coded for in the vector as identified. If an CD27+ IgG1+ memory B-cell or other CD+ IgG1 memory B-cell is identified to have potent binding to the target of interest not only will an expression vector encoding an IgG1 immunoglobulin for precisely the same immunoglobulin heavy and light chains as identified in the IgG1+CD27+ memory B-cell. Additionally, the V_{H}-region of the immunoglobulin heavy chain may be paired with the constant domains (C_{H}1, hinge, C_{H}2 and C_{H}3) of another isotype while leaving the immunoglobulin light chain unchanged. With the idea that increased binding affinity can occur simply as a result of an isotype switch from IgM or IgD to IgG, IgA or IgE without changing the amino acid sequence of the V-region. Further, the constant regions of each class and subclass have different effector functions. Additionally, enhanced or reduced signal transfer from Fab to the Fragment crystallizable (Fc) region to the can occur simply as a result of isotype switching (See e.g., Pritsch, O., et al., 1996, J Clin Invest. 98:2235-2243.; Janda, A., et. al., 2016, Front Microbiol., 7:22). Not only will immunoglobulins V-regions be investigated for different isotype functions but they will be investigated for their Fab and F(ab′)2 domains in a variety of ways as described in this instant patent application. Although, the general emphasis is on dIgA1.

### 4. Incorporation of the Immunoglobulin Genetic Information of IgG1, IgG2, IgG3, IgG4, IgA1, IgA2, dIgA1 or dIgA2 into Non-Viral, Viral or Retroviral Expression Vectors Intended for Different Delivery Vehicles Such as Adeno-Associated Virus, Adenovirus, Lentivirus or Vesicle Based Delivery Systems to Enable a Polyclonal Expression of Immunoglobulins

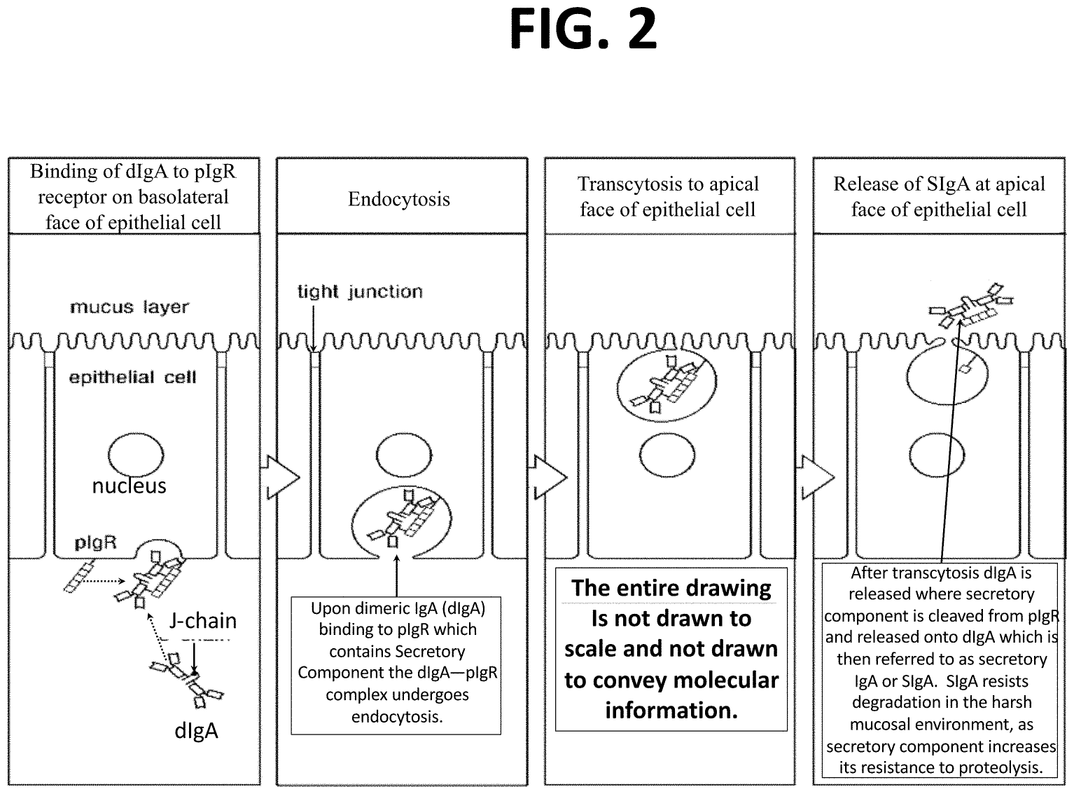

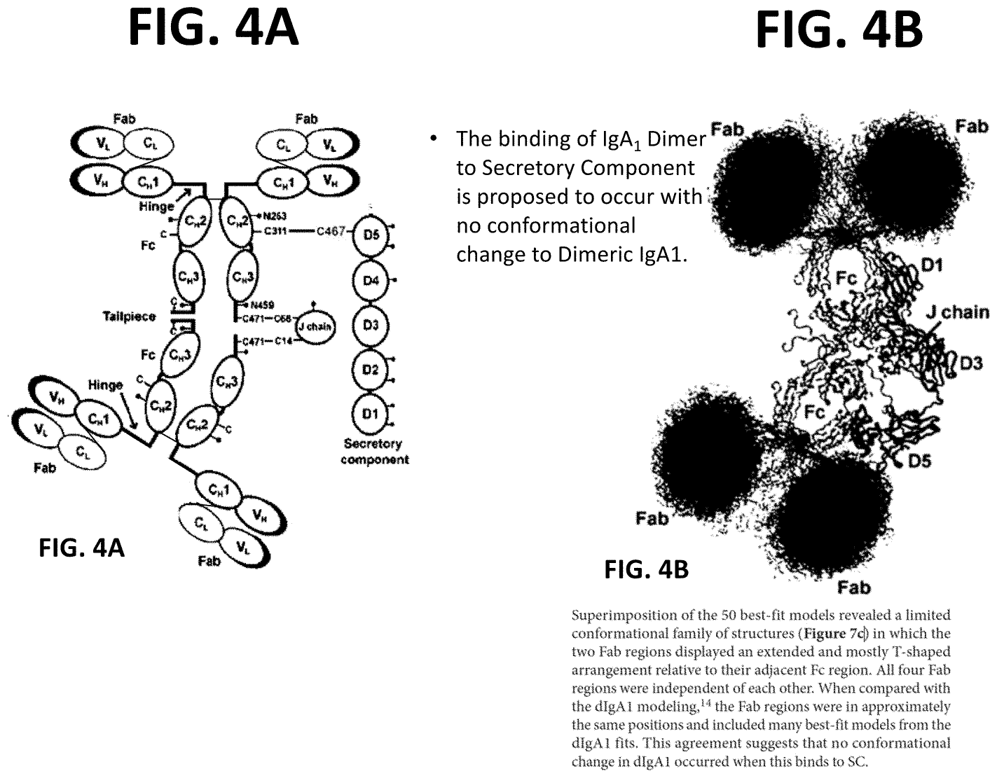

In the invention the immunoglobulins identified to both have “therapeutic binding affinity” are intended to be expressed from episomal DNA in the host cells. AAV, lentivirus, vesicle based delivery systems will be used to transport the vector constructs to their target cells with destination of the nucleus of the target cell. In the invention A mix of episomes each of which codes for a unique immunoglobulin and collectively which code for a polyclonal mix of immunoglobulins. As an example, there may be a total of 3 unique immunoglobulins that are coded for by single stranded DNA in an AAV, double stranded DNA in an adenovirus, mRNA or DNA in a vesicle based delivery system, or mRNA in a lentivirus. Those immunoglobulins will include one or more of the following immunoglobulin classes: dIgA1 and dIgA2, IgG1, IgG2, IgG3, IgA1, IgA2, (See FIG. 3). In one embodiment a single immunoglobulin may be used as well as part of a monoclonal antibody expression from an episome. Mucosal immunity against the pathogen of interest is best achieved with SIgA1 or even SIgA2 that is a product of dIgA1 or dIgA2 respectively following transcytosis from the basal to apical face of an epithelial cell where dIgA1 forms a disulfide bond (See FIG. 4A) with secretory component that is found on polymeric immunoglobulin secreting receptor (pIgR) as part of crossing the epithelial cell (See FIG. 2). It is proposed that dIgA1 would provide more effective mucosal immunity over dIgA2 in cases where the hinge is not heavily subjected to cleavage by proteolysis such as in the digestive tract; although, SIgA1 can function effectively in the digestive tract but is more prone to cleavage than SIgA2. IgA1 has a 19 amino acid hinge length, which affords great flexibility between the Fab and Fc region and also allows for a more rapid rate of agglutination between multiple virions, bacteria, allergens, or any binding target with multiple binding faces and the secretory immunoglobulin A1 (SIgA1). In the absence of secretory component the hinge of IgA1 and dIgA1 is readily cleaved by proteases in the mucus where there are multiple cleavage sites on the hinge where the 6 amino acid hinge of IgA2 and dIgA2 do not have any cleavage sites in the hinge and thus would not be degraded as quickly in the mucus. Although, SIgA1 is not expected to be cleaved at the hinge by proteases in the harsh environment of the mucus as because secretory component effectively blocks proteases from accessing cleavage sites on the dIgA1 part of SIgA1 and also the dIgA1 part of SIgA1 blocks proteases from accessing cleavage sites on secretory component. IgA2 has a 6 amino acid hinge length, which protects it from proteases but does not afford it a high degree of flexibility. (See e.g., Bonner, A. et. al., 2009, Mucosal Immunol 2:74-84; Kumar, S., et al. bioRxiv 2020.02) This short hinge length of dIgA2 makes it more difficult to achieve agglutination with 4 targets that a single dIgA1 is capable of where dIgA2 is more likely to achieve agglutination with two binding targets of the dIgA2 immunoglobulin. Another challenge accorded with the short hinge of dIgA2 is the lack of flexibility between the Fab and the Fc region making this immunoglobulin more constrained in sampling the immediate vicinity of nearby binding targets. Although, in the gut mucosa because the environment is even more harsh than other mucosal environments SIgA1 could be more subjected to cleavage than SIgA2. In further embodiments of the invention a hinge longer than dIgA2 but shorter than dIgA1 is considered. In additional embodiments a hinge longer than that of dIgA1 is considered. In further embodiments replacing one or more of the amino acids that make up the hinge of dIgA1 or dIgA2 with another amino acid is considered.

Immunoglobulin constant regions can have isotypic variants known as allotypes which are generally substitutions of one or more amino acids between allotypes. For example the constant region of the IgA2 immunoglobulin heavy chain has two allotyes referred to as A2m(1) and A2m(2). A2m(2) differs from A2m(1) in that there are 23 amino acid substitutions in addition. IgA1 on the other hand does not have allotypes and also unlike both allotypes of IgA2, IgA1 does not have a disulfide bond between the immunoglobulin light and heavy chains. The immunoglobulin Kappa light chain also has three allotypes Km(1), Km(2) and Km(3) that differ by only one or two amino acids between each allotype. Although interestingly Km(3) is the most commonly used allotype in monoclonal antibodies as some reports cite enhanced specificity from Km(3) over Km(2) and Km(1) where Km(3) has alanine at position 153 and valine at position 191. The Lamba light chain also does not have allotypes discovered in nature. There are several reports that speak of potential immunogenicity from using natural allotypes in therapeutics and even in convalescent plasma and blood transfusions. Although, there is definitive reports of anti-allotype responses patients do routinely express anti-therapeutic antibodies. Although, these antitherapeutic antibodies could be the result of the V-regions and even more specifically the CDR regions rather than the constant regions of the immunoglobulin. (See, e.g. Toraño, A., & Putnam, F. W., 1978, Proceedings of the National Academy of Sciences of the United States of America, 75:966-969.; Li, Z., 2013, Exp Hematol Oncol 2:6)

Immunoglobulins are dimers of heterodimeric proteins that consist of light and heavy chain proteins of moderate size linked together through disulfide bonds and thus, they can be difficult to express from adeno-associated virus (AAV) viral vector constructs due to the AAV capsid limited packaging capacity. Reports of the expression of immunoglobulin vector constructs have focused on the expression of immunoglobulins that are dimers of the heterodimers. That is the immunoglobulin consists of two identical heavy chains and two identical light chains linked together through disulfide bonds. One reason immunoglobulins can be difficult to express in vector constructs due to the limited capacity of the vectors. For example, the maximum capacity of an Adeno-Associated Virus (AAV) capsid is typically about 4.9 kilobases of single-stranded DNA. Typically, to express two gene elements from a single vector construct there must be enough room for both DNA sequences, a promoter that includes a ribosome binding site, an intermediate promoter between the two gene elements that includes a ribosome binding site, a polyadenylation element (e.g. Simian Virus 40 polyadenylation element (SV40 polyA) (For an example see SEQ ID NO. 4) or Bovine Growth Hormone polyadenylation element (BGA polyA)) and potentially a post transcriptional regulatory element (e.g. Woodchuck Hepatitis Virus (WHP) Posttranscriptional Regulatory Element (WPRE). In this context collectively, the sum of these elements approaches or exceeds the 4.9 kilobase capacity of the AAV vector or in the case of an IRES the expression of the section transgene is much lower. Although, in addition even if there is enough room to house the two genes with separate promoters and regulatory elements the expression level of one of the two genes if not both genes is often substantially reduced resulting in a substantial excess of one of the two immunoglobulin chains. There have been a few reports and patents that address the challenge associated with obtaining sufficiently high expression of immunoglobulins that are dimers of heterodimers.

There are two major strategies that may be used. The first is to express the immunoglobulin light and heavy chains as a single open reading frame. When two genes of the immunoglobulin light and heavy chains are expressed as a single open reading frame (that is the stop codon of the upstream gene is not encoded) a furin cleavage site and a 2A self-cleaving peptide are used to ensure efficient yields and viable immunoglobulin production. (See e.g., U.S. Pat. Nos. 7,498,024 B2 and 7,709,224 B2 expressly incorporated by reference herein in their entirety). Alternatively, a 2A self-processing peptide placed between two consecutive transgenes encoded for in a single open reading frame may be used in the absence of the furin cleavage site.

In one embodiment A furin cleavage site is used in conjunction with the 2A self-cleaving peptide to eliminate the large polypeptide residue that would otherwise be left on the immunoglobulin C terminal end on the upstream immunoglobulin chain after self-cleavage or self-processing of the 2A self-cleaving peptide. Furin cleavage sites of four amino acids in length have a consensus sequence of (N-terminus)—RXKR—cleavage point—(C-terminus)— where X can be any amino acid—and furin cleavage sites range from 4 to 6 peptides in length and are cleaved by furin or other proteases. If the furin cleavage site is located C-terminus to a transgene as part of a single open reading frame in a nucleic acid vector the expression of the transgene will occur with a 4-6 amino acid furin cleavage site residue on the C-terminal end of the resulting protein. Additionally, a 2A self-processing or self-cleaving polypeptide has a consensus sequence following cleavage of (N-terminus)—D(X)E(X)NPG—cleavage point/ribosomal skip—P—(C-terminus). Thus, if a 2A self-processing peptide is followed by a transgene as part of a single open reading frame the expression of the transgene will occur with a 1 amino acid (Proline) 2A self-processing peptide residue on the N-terminal end of the resulting protein. However, if there is a leader sequence or signaling peptide that is cleaved from the protein as part of processing such as transporting the protein outside the nucleus then there would be no 2A cleavage or ribosomal skip residue left on the N-terminal end of the functioning protein. It has been demonstrated that the cleavage residue of 2A self-cleaving polypeptide are efficiently removed from the C-terminus of the adjoining upstream immunoglobulin chain by placing a furin cleavage site directly upstream of the 2A residue resulting in an immunoglobulin chain with a furin cleavage site on the C-terminus end following by the 2A self-cleaving peptide directly C-terminus to the furin cleavage site. (See e.g., Fang, J., et al., 2005, Nat. Biotechnol., 23: 584-590.; Krysan, D. J., et. al., 1999, J Biol Chem. 274:23229-23234; U.S. Pat. No. 7,498,024).

An alternative and earlier reported strategy utilized a dual-promoter rAAV vector to express immunoglobulins. CMV and SV40 small T-antigen intron was used as the promoter for the first transcriptional unit that was followed by the leader sequence (or signal peptide) and heavy chain sequence. The leader sequence is a 19 amino acid cleavable sequence that is expressed N-terminal on the heavy (Leader Sequence Mouse: MGWSCIFLFLLSVTVGVFS) (Most common Leader Sequence Human: MDWTWRILFLVAAATGAHS) and light chain (Among most common Leader Sequence found on Kappa light chains MDMRVPAQLLGLLLLWLPG) chain immunoglobulins and is necessary for efficient translocation into the endoplasmic reticulum (For a list of Leader sequences found in human see Table 1). A Bovine Growth Hormone (BGH) polyadenylation (polyA) (For an example see SEQ ID NO. 5) site was used as the post-transcriptional regulatory element for the first transcriptional subunit. The second transcriptional unit utilized a human elongation factor 1α (EF1-a) promoter that was modified to improve stability of DNA and RNA that also contained an intron 1117 (SEQ ID NO. 9) 5′Untranslated Region (5′UTR). An SV40 polyadenylation site was used as the post-transcriptional regulatory element for the second transcriptional subunit. (See e.g., Lewis, A. D., et. al., 2002, J. Virol. 76:8769-8775; Huang, M. T., et. al., 1990, Molecular and cellular biology, 10:1805-1810; Wu, C., et. al., 2004, Molecular and cellular biology, 24:2789-2796).

Previously, these two considered vector constructs were used to express immunoglobulins that are dimers of hetero dimers in mammalian models. (See e.g., Fang, J., et al., 2005, Nat. Biotechnol., 23: 584-590; Lewis, A. D., et. al., 2002, J Virol. 76:8769-8775) However, these reports were not concerned with the expression of dimeric immunoglobulins (dIgA), which only exist for isotype A in human. Dimeric immunoglobulins are tetramers of heterodimers and requires J chain for efficient formation. No such reports or patents concerned with the expression of dIgA from a single AAV vector nor multiple AAV vectors have been identified, nor have such reports been identified for the expression of dIgA from a lentiviral vector nor any vector including an mRNA vector.

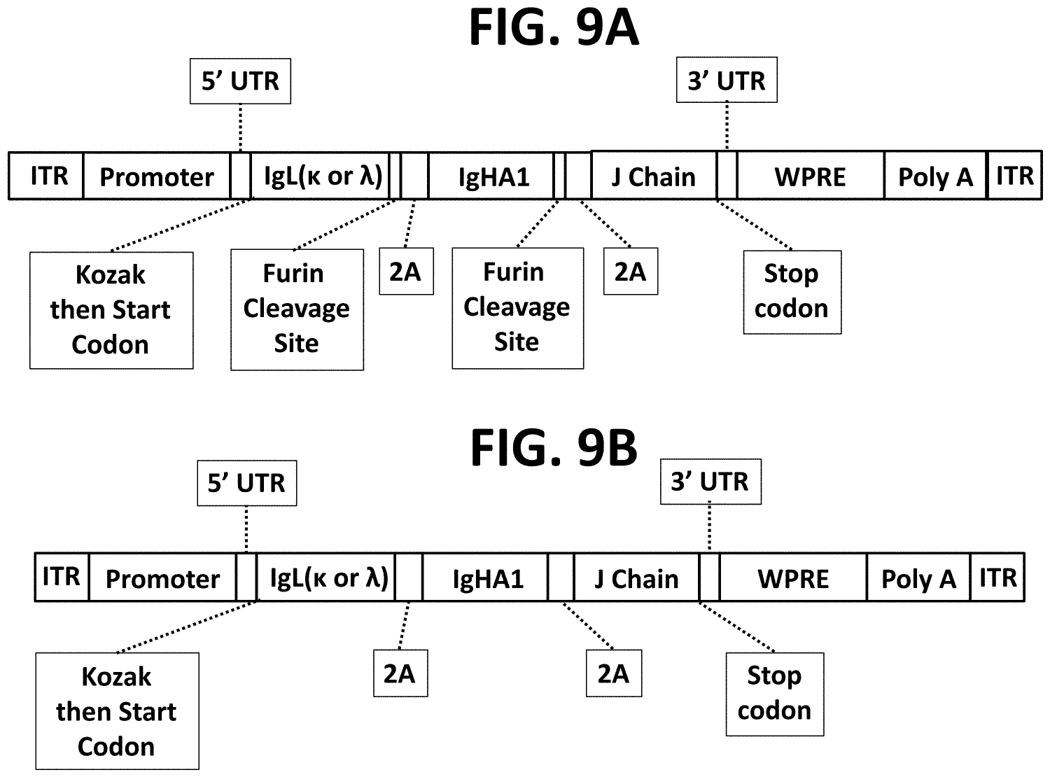

J chain a 159 amino acid (SEQ ID NO. 11) protein with peptide signaling leader sequence MKNHLLFWGVLAVFIKAVHVKA (SEQ ID No. 27) that is cleaved to a 137 amino acid protein (SEQ ID NO. 7) is shown to be required for efficient production of dIgA. (See e.g., Lycke, N., et. al., 1999, J. Immunol. 163:913-919; Sorensen, V., et. al., 1999, J. Immunol., 162:3448-3455; Schroeder, H., et. al., 2008, Fundamental Immunology (Book) 6^{th}:125-151; Castro, C. et. al., 2014, Journal of Immunology, 193:3248-3255; Koshland, M. E., 1985, Annu Rev Immunol. 3:425-453) Without J chain there is a substantial reduction in the amount of dIgA produced by an organism such as mice. J chain forms 2 disulfide bonds (See FIG. 9) with two IgA1 s at the penultimate cysteine (C471) on the heavy chain tail of each IgA1 and C14 and C68 of J chain. J chain has been called the glue for the efficient formation of dIgA. (Bonner, A. et. al., 2009, Mucosal Immunol 2:74-84) J chain has also been shown to be necessary for binding to secretory component on pIgR. (See e.g., Johansen, F. E., et. al. 2000, Scandinavian Journal of Immunology., 52:240-248) The partially buried ligand binding motifs of secretory component are thought to interact with J chain of Dimeric IgA as part of inducing a conformational change to free domain 5 of secretory component on pIgR and form a disulfide bond between C467 of secretory component and C_{H}2 cysteine C311 of the immunoglobulin heavy chain of dIgA. (See e.g., Wang, Y., Wang, G., Li, Y. et al., 2020, Cell Res 30:602-609)

In some embodiments Marginal Zone B1 Cell Specific Protein (MZB1) (SEQ ID NO. 8) is co expressed in the vector not targeting B-cells, where it is naturally expressed in humans, for more efficient formation of some allotypes of dIgA2. MZB1 Double Knockout (MZB1^{-/-}) mice produce significantly lower quantities of dIgA than mice that have a functioning MZB1 gene. (See e.g., Xiong, E., et. al., 2019, Proceedings of the National Academy of Sciences, 116:13480-13489) In mice without the availability of MZB1 and J chain proteins dIgA would form in very small quantities and would fail to convert over to SIgA. MZB1 is proposed to interact with IgA through the α-heavy chain (αHC) tailpiece dependent on the penultimate cysteine residue (where two αHCs form a disulfide bond with J chain cysteine C14 and C68) and prevents intracellular degradation of α-heavy chain—Light chain (αHC-LC) IgA Dimer complexes. MZB1 promotes J chain binding to IgA.

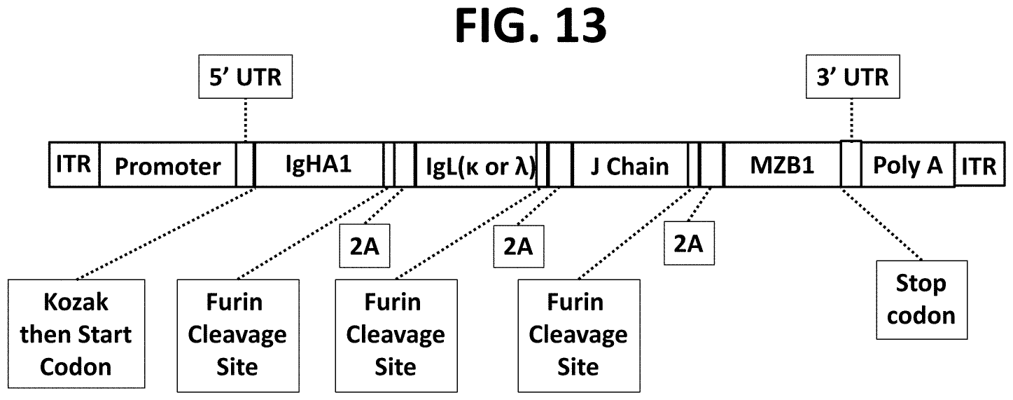

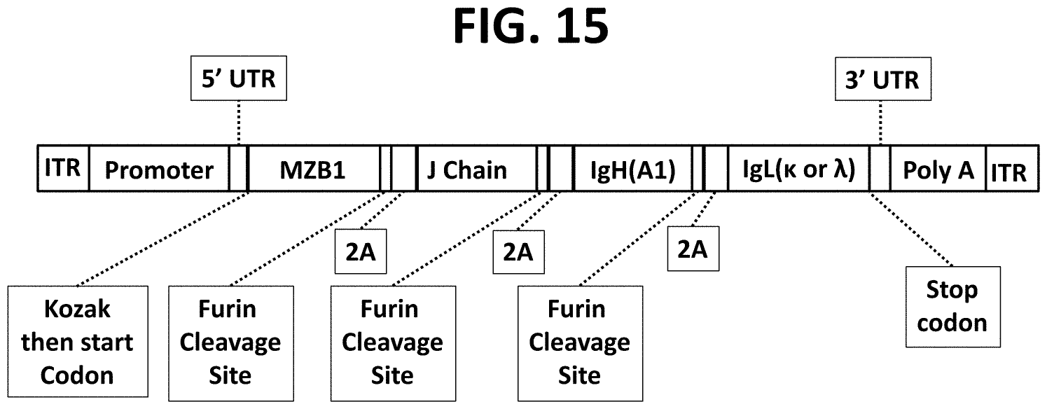

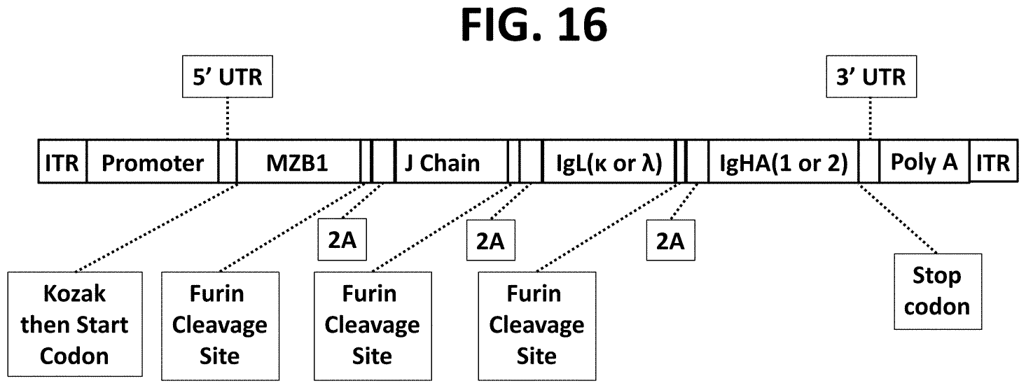

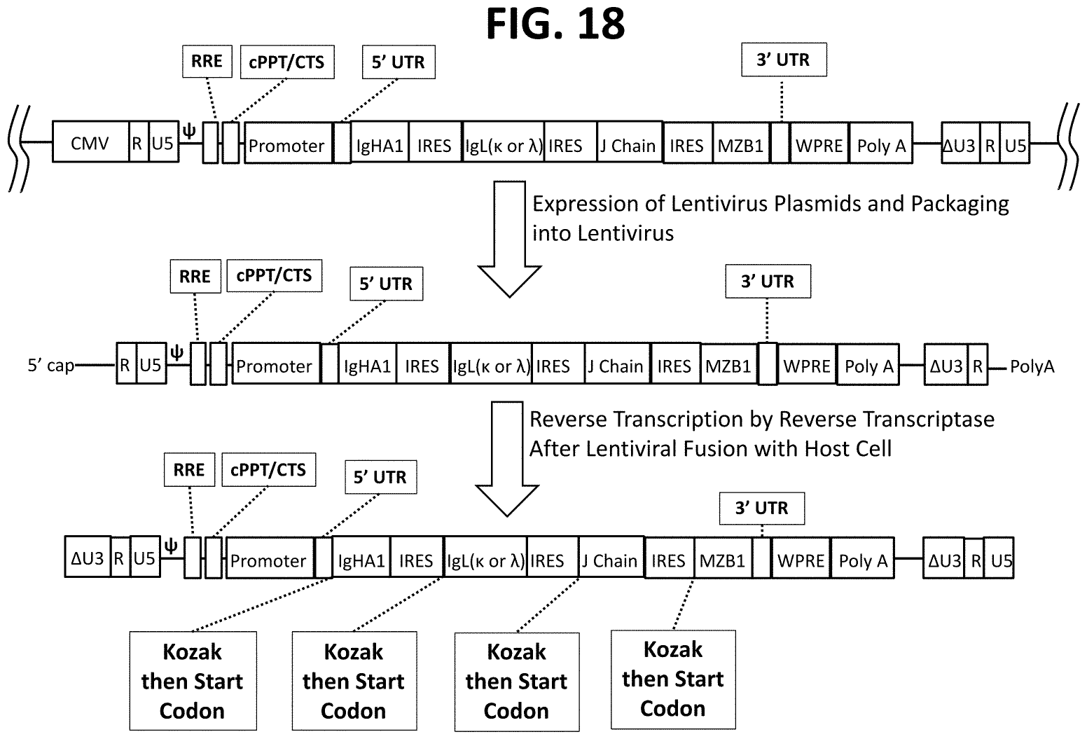

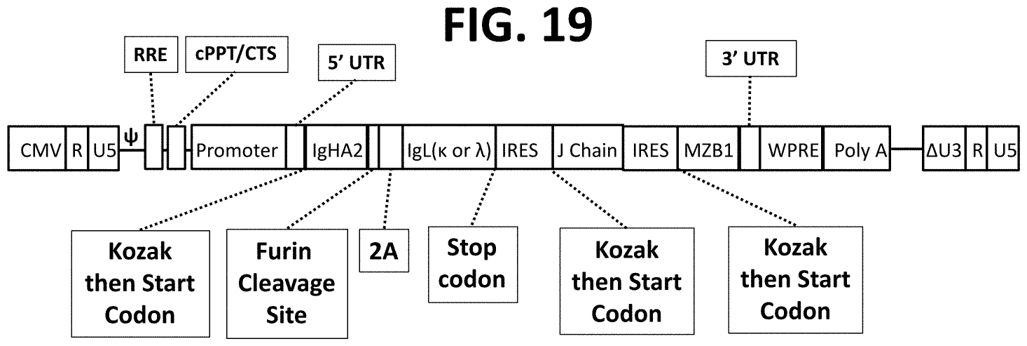

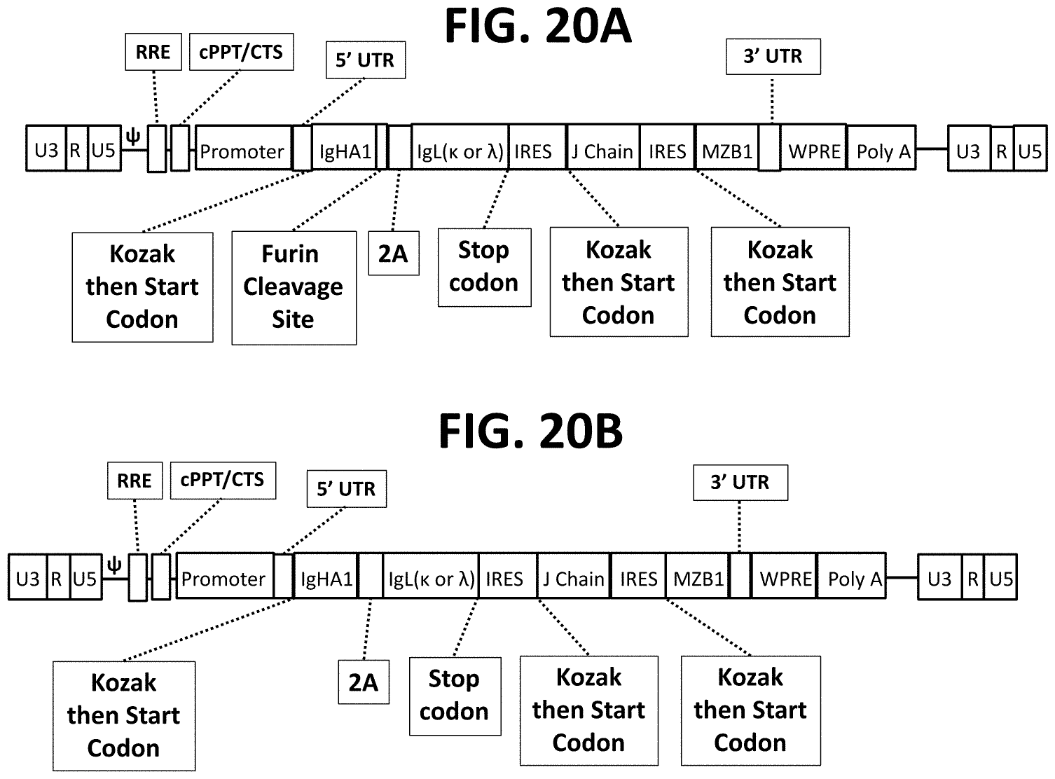

Additionally, this instant patent optionally includes the expression of dIgA with MZB1 (See FIGS. 13, 15, 16, 17, 18, 19, and 20 as examples) and without encoding for MZB1 in the non-viral vector, viral vector or retroviral vector. In some vector constructs dIgA expression may occur through the incorporation of IgH, IgL and J chain in the vector in the absence of encoding for MZB1. Optionally, J chain expression will be encoded in the non-viral vector, viral vector or retroviral vector to occur with the signaling polypeptide (leader sequence) in some constructs and will be encoded in the vector without the signaling polypeptide (leader sequence) in other embodiments.

### Immunoglobulin Regions

Human immunoglobulins are complex proteins with different structural/functional regions. FIG. 3 depicts the different structural/functional regions of immunoglobulins. The domain of the immunoglobulin responsible for binding to the antigen of interest is referred to as the antigen-binding fragment (Fab) domain. The Fab domain is considered to be made up of the entire immunoglobulin light chain (IgL) that includes its V-region (V_{L}) domain and constant region domain (C_{L}) and the immunoglobulin heavy chain's V-region (V_{H}) domain and C_{H}1 domain. Researchers may also classify the Fab to include the hinge domain of the heavy chain and refer to it as F(ab′)2 fragment. Both the Fab and F(ab′)2 domains are considered as part of this patent filing. It is reasonably likely that one may be able to use the Fab or F(ab′)2 fragments of an immunoglobulin with high affinity for an interested and shown in many cases that making some modification to the crystallizable fragment Fc (made up of all peptides that include the hinge, C_{H}2, and remaining domains C-terminal to C_{H}2) and pFc′ (made up of all peptides that include C_{H}2 and remaining domains C-terminal to C_{H}2) and show there is little if any change in affinity of the Fab and F(ab′)2 for their antigen. One may modify or change such Fc and pFc′ regions by (A) changing from another human coding source for the same subclass—to also avoid the potential of an immune response against the Fc and pFc′ regions. (B) Alternatively, one may engineer the Fc and pFc′ regions on IgG immunoglobulin to minimize their binding to the FC receptors such as FcαRI in order to minimize antibody dependent enhancement of infection. (See e.g., Iwasaki, A. & Yang, Y., 2020, Nat. Rev. Immunol. 20:339-341). (C) One may also engineer the Fc and pFc′ regions on any immunoglobulin to improve their immunotolerance. (D) One may incorporate IgA1 and IgA2 constant regions and immunoglobulin light chain constant regions IgL(κ or λ) into expression vectors that express V_{H} and V_{L} respectively as identified from scFv identified in phage display libraries and also co-express J chain (with optional co-expression of MZB1) to enable expression of dIgA1 and dIgA2. Additionally, this patent considers SIgA-virion polymerization or SIgA-bacteria polymerization to be an effective potential means of virus neutralization in the respiratory mucosa. (see e.g., Terauchi, Y., et. al., 2018, Human vaccines & immunotherapeutics, 14:1351-1361.). During affinity maturation B-cells can alter their immunoglobulins by both somatic hyper mutation of the DNA coding for the V_{H} and V_{L} domains. Additionally, during affinity maturation B-cells can alter their immunoglobulins through isotype switching that is through switching the entire constant region polypeptide. This isotype switching can result in changing the Cμ constant region with any downstream constant region. All immunoglobulin class switch recombination events occur from IgM or IgD to IgG, IgA or IgE. For example, IgG to IgA class switching is not reported to be observed. (See e.g., Stavnezer, J., et al., 2014, Journal of immunology, 193:5370-5378.) However, class switching can see an observed increased in affinity. The rational for pairing the V-region of one Ig isotype with that of another is that increased binding affinity can occur simply as a result of an isotype switch from IgM or IgD to IgG or IgA without changing the amino acid sequence of the V-region. Additionally, enhanced or reduced signal transfer from Fab to Fc can occur simply as a result of isotype switching (See e.g., Janda, A., et. al., 2016, Front Microbiol. 7:22).

### Engineering Fc Region of Immunoglobulin to Prevent Antibody Mediated Viral Enhancement or to Improve Other Effector Functions

Engineering the Fc region of immunoglobulins can serve a large variety of functions including altering their half-life, to enhance complement dependent effector function, to enhance or reduce Fc receptor effector functions and to enhance binding affinity of immunoglobulins. Enhancing complement dependent effector functions as enhancing the complement effector functions of an immunoglobulin leads to more rapid response by supporting immune cells and immune signaling proteins allowing the pathogen to be eliminated more rapidly. In cases where antibody cocktails are used and including immunoglobulins other than dIgA there is an inherent probability of cytokine storm with some classes of IgG immunoglobulins binding to some antigens or even potentially allergens as a recent example if the epithelial barrier is not breached. However, should some pathogens breach the epithelial barrier the probably of cytokine storm is significantly higher. In some cases immunoglobulins may target pathogen cell surface proteins and prevent interactions between cell surface proteins and host receptors.

It may be beneficial to reduce or eliminate effector function to prevent cytokine secretions and the secretion of other pro-inflammatory agents that occurs with antibody mediation viral enhancement or antibody dependent enhancement (ADE) where it is possible for pathogen specific antibodies to promote pathology in the lung tissue, urinary tract, reproductive tract and even potentially the stomach without infecting the epithelial lining. Generally, this only occurs when antibody levels specific to the target are low or antibody-binding constants are not high enough and are thus non-neutralizing. This instant patent aims to identify those antibodies developed by humans that are of a high binding affinity. However, in the case where the most potent natural antibodies against the target protein falls below a specific threshold value or below the optimal “therapeutic binding affinity” one may consider engineering Fc fragments and C_{H}1 and C_{L} domains to reduce ADE. ADE occurs mostly due to the interactions between the antibody and Fc gamma receptors (FcγRs) which IgA does not have, rather IgA is detected by Fc alpha receptors (FcαRI) where ADE does not happen as readily as with IgG. ADE may be mediated by the binding of the Fc regions of antigen-bound or allergen-bound antibodies to immune cells. One may also change or engineer the immunoglobulin constant light (C_{L}) domains by adding a furin cleavage site or 2A self-processing peptide to their C-terminal end. These changes might interfere with Fcγ receptor binding to the immunoglobulin C_{H}2 constant region. (See e.g., Lu, J., Sun, P. D., et al. 2015, Proceedings of the National Academy of Sciences of the United States of America, vol. 112, pp. 833-838.) When an immunoglobulin binds to an antigen there is a conformational change in the immunoglobulin that causes the C_{H}1 and C_{L} domains of the Fab to undergo a conformational change and engage the Fc region. (See e.g., Pritsch, O., et al., 1996, J Clin Invest. 98:2235-2243; Janda, A., et. al., 2016, Front Microbiol., 7:22; Iwasaki, A. & Yang, Y., 2020, Nat. Rev. Immunol. 20:339-341;.). ADE occurs due to the interactions between the antibody and Fc receptors including Fc gamma receptors (FcγRs). ADE may also be mediated by the binding of the Fc regions of protein-bound antibodies to immune cells. ADE may occur in the lumen of the lungs without the pathogen breaching the epithelial lining. Internalization of the antibody bound to its target in the lungs has the potential to promote inflammation and tissue damage through increased levels of pro-inflammatory chemokines CCL2 and CCL3 and reduced levels of decreased levels of the anti-inflammatory cytokines IL-10 and TGFβ. CC chemokines such as CCL2 and CCL3 promote the migration of monocytes to the lung. CCL2 and CCL3 attract monocytes through the receptor CCR2B, inducing their migration from the bloodstream to become tissue macrophages. SIgA1 and SIgA2 are not efficient activators of the complement cascade that is involved in cytokine storm nor are they efficient activators of proinflammatory responses but rather are anti-inflammatory in nature. This is a result of partial blockage of the FcαRI by secretory component preventing efficient access by resident macrophages, monocytes, neutrophils and eosinophils of the myeloid lineage that are often resident innate immune cells in the mucosa and exocrine lumen that decreases the chance of activating the complement cascade and initiating damaging inflammation. For example, opsonic activity of SIgA is poor compared with dIgA or IgA this is due to partial blockage of the FcαRI binding site by secretory component. This is consistent with the more anti-inflammatory role of SIgA. In contrast dIgA and IgA have a dual role in immunity providing both anti-inflammatory and proinflammatory roles. (See e.g., F. et al., 2008, J. Immunol., 181:6337-6348; Iwasaki, A. & Yang, Y., 2020, Nat. Rev. Immunol. 20:339-341.; Yip, M. S. et al.; 2016, Hong Kong Med. J. 22:25-31; Wang, S. F. et al., 2014, Biochem. Biophys. Res. Commun. 451:208-214; Mak, T., 2014, Primer to the Immune Response 2nd Ed:269-292; Bakema, J., van Egmond, M., 2011, Mucosal Immunol., 4:612-624)

IgA, dIgA and SIgA all variants of IgA are unique in that they each are capable of a different role in inflammation including antibody mediated cellular cytotoxicity, proinflammatory, anti-inflammatory responses. Crystallographic studies have established that one IgA can simultaneously bind to two FcαRI molecules on the surface of the myeloid derived innate immune cell. Crosslinking of FcαRI by an IgA immune complex induces FcRγ-chain independent redistribution of FcαRI to plasma membrane rafts (glycosphingolipids and cholesterol. These domains, termed membrane “rafts,” have estimated average sizes ranging from 70 nm). Monomeric IgA in unbound form inhibits the activation of heterologous receptors (e.g. other FcRs, cytokine receptors, chemotactic receptors and TLRs) For example, experiments with eosinophils, monocytes and transfectants demonstrated that FcαRI shows low capacity to interact with IgA immune complexes in a resting state, but ligand binding capacity increases profoundly after stimulation with cytokines such as granulocyte-macrophage-colony stimulating factor (GM-CSF) and IL-4 or IL-5. The ambivalent functions of IgA in human cells are regulated by the CD-89 associated FcRγ chain. dIgA while involved in proinflammatory responses on the other hand is not capable of crosslinking two receptors on an innate immune cell with FcαRI thus is not able to promote the same level of inflammatory response as a crosslinked IgA1 in an immune complex. dIgA like IgA1 has a fully exposed region to bind to FcαRI receptors found on myeloid lineage monocytes, macrophages, neutrophils and eosinophils. When in an immune complex dIgA triggers proinflammatory responses in these cells with the FcαRI receptors it can trigger opsonization and phagocytosis in innate immune cells such as neutrophils with an FcαRI receptor. Tissue distribution of FcαRI is mostly defined by the presence of neutrophils and some macrophages SIgA has partial blockage of FcαRI by secretory component this dampens the proinflammatory response and favors an anti-inflammatory response. Additionally, only a few FcαRI positive cells are observed in mucosal areas in homeostatic conditions. For instance, intestinal macrophages lack FcαRI expression which is consistent with an anti-inflammatory role of SIgA to protect mucosal immunity. Langerhans cells (tissue resident macrophages of the skin) do not express FcαRI. The role of SIgA is to preserve the epithelium in the mucosa and ducts. SIgA binding by FcαRI is increased when complement receptor 3 (CR3) functions as a coreceptor. (See, e.g. Bakema, J., van Egmond, M., 2011, Mucosal Immunol, 4:612-624; Ben Mkaddem, S., Benhamou, M., & Monteiro, R. C., 2019, Frontiers in immunology, 10:811.)

### Minimizing Immunogenicity of the Vector Design

The introduction of foreign oligopeptides greater than 8 amino acids is capable of eliciting an immune response in individuals. Most nucleated non-lymphocyte cells express the major histocompatibility complex class I (MHCI) which primarily bind oligopeptides 8-14 amino acids in length generated from degradation of cytosolic proteins. B-cells and T-cells on the other hand present major histocompatibility complex class II (MHCII) that present 13-25 amino acid oligopeptides where there is an increasing affinity between the MHCII and a 16-20 amino acid oligo peptide where there is an order of magnitude reduction in binding affinity for the oligopeptide to the MHCII for each amino acid reduction in oligopeptide length below a 16 amino acid oligopeptide. (See, e.g. O'Brien, C., Flower, D. R., & Feighery, C., 2008, Immunome research, 4:6.) T-cell receptors (TCR) on CD8+ Cytotoxic T-cells detect the oligopeptides presented in MHCI to determine if they are self-antigens or not. Although TCR on CD4+ helper T cells detect the oligopeptides presented in MHCII to determine if they are foreign in nature and if they are determined foreign the B-cell is activated. Thus, B-cells which do not have a CD8+ cytotoxic T-cell mechanism are quite well suited to express the integration competent lentiviral gene therapy. Hepatocytes present MHCI but virally infected hepatocytes are largely resistant to perforin/granzyme-mediated killing by CD8+ cytotoxic T-cells. In mouse and chimpanzee models of Hepatitis B (HBV) infection IFN-γ produced by virus specific CD8+ T-cells correlates with a profound reduction in serum and liver HBV DNA by disrupting viral capsid integrity and helps to reduce hepatocyte damage by inducing cyto-protective proteins that confer resistance to granzyme B-mediated killing. (See, e.g. Gehring, A. J., Sun, D., M., Bertoletti, A., et al., 2007, J Virol., 81:2940-9.) Thus, hepatocyte expression of the gene therapy will be short lived if CD8+ T-cells recognizes non-self antigen peptide sequences. The potential sequences in the antibody gene therapy that could be recognized by T-cells include 2A self-processing peptide fragments. There is there is research that demonstrates that E2A peptide fragments will not elicit a CD8+ cytotoxic T-cell response. In an ex vivo research T-cells from 16 immunocompetent human donors were subjected to an ex vivo culture system to measure whether an ex vivo T-cell response could be elicited against the Thosea Asigna Virus (TAV) 2A self-processing peptide (T2A) or 2A-equine rhinitis virus (ERAV) E2A. The ex vivo culture system was one that was previously validated to induce T cell responses even against weakly immunogenic antigens. Of the 16 donors tested ex vivo 5 released very low levers of IFN-γ in response to T2A peptide mixtures and none produced cytotoxic activity or responded to E2A. (See, e.g. Arber, C., Savoldo, B., et al. 2013, Gene therapy, 20:958-962) This, research supports for the use of hepatocytes to express dIgA.

TABLE 1
Table of Human Signaling Peptides
(Most Common Bolded)

| SEQ | Signaling | | |
|---|---|---|---|
| ID NO: | Peptide | Length | Protein |
| 27 | MKNHLLFWGVL | 22 | J Chain |
| | AVFIKAVHVKA | | |
| 28 | MDWTWRILFLV | 19 | Immunoglobulin Heavy |
| | AAATGAHS | | Variable |
| 29 | MDTLCSTLLLL | 19 | Immunoglobulin Heavy |
| | TIPSWVLS | | Variable |
| 30 | MDMRVPAQLLG | 19 | Immunoglobulin Kappa |
| | LLLLWLPG | | Variable |
| 31 | MDMMVPAQLLG | 19 | Immunoglobulin Kappa |
| | LLLLWFPG | | Variable |
| 32 | MRVPAQLLGLL | 20 | Immunoglobulin Kappa |
| | LLWLPGARC | | Variable |
| 33 | MRLPAQLLGLL | 19 | Immunoglobulin Kappa |
| | MLWVPGKD | | Variable |
| 34 | MRLLAQLLGLL | 19 | Immunoglobulin Kappa |
| | MLWVPGSS | | Variable |
| 35 | MAWTPLWLTLL | 19 | Immunoglobulin Lambda |
| | TLCIGSVV | | Variable |
| 36 | MAWTPLFLFLL | 19 | Immunoglobulin Lambda |
| | TCCPGSNS | | Variable |
| 37 | MAWALLLLTLL | 19 | Immunoglobulin Lambda |
| | TRDTGSWA | | Variable |
| 38 | MPWALLLLTLL | 19 | Immunoglobulin Lambda |
| | THSAVSVV | | Variable |
| 39 | MAWSSLLLTLL | 19 | Immunoglobulin Lamba |
| | AHCTGSWA | | Variable |
| 40 | MAWSPLFLTLI | 19 | Immunoglobulin Lamba |
| | THCAGSWA | | Variable |
| 41 | MIYEVSHRPSG | 19 | Immunoglobulin Lambda |
| | VSTRFSAS | | Variable |

Skeletal muscle cells do not constitutively express or display WIC class I molecules, although they can possibly be induced to do so by proinflammatory cytokines such as IFN-γ and tumor necrosis factor α (TNFα). Although, muscle cells themselves cannot be made to produce IFN-γ nor TNFα under stimulating conditions consistent with gene therapy production of proteins and DNA based immunizations. Also, it was shown that overexpression of MHCI in muscle cells that is observed in some muscular and neuromuscular disorders may be the event that leads to autoimmune disorders of the skeletal muscle such as idiopathic inflammatory myopathies. Muscle has proved resistant to the development of inflammation caused by immunization with heterologous muscle, and inflammation does not persist long after acute viral injury. Although, research has shown that sufficient proinflammatory stimuli in muscles cells of mice induces the expression of other cytokines such as IL-6, transforming growth factor-beta (TGF-β), and granulocyte-macrophage colony-stimulating factor (GM-CSF) by muscle cells themselves, as well as the up-regulation of MHCI and MHCII. Although, none of IL-1α, IL-4, IL-10, IL-12 or IFN-γ synthesis by human skeletal myoblasts was detected in reports. Further, Cytokine proteins for IFN-α, TNF-α, MIP-1α were not detected to be expressed by muscle cells in studies. Consequently, muscle is considered an attractive target for gene therapy and for the administration of DNA based immunizations speaking to their robustness for their purpose for most individuals in the population. Thus, administering the mRNA, AAV and non-integrating DNA based gene therapies into skeletal muscle cells for the expression of dIgA is an attractive target for individuals that do not have neuromuscular disorders, muscular disorders nor constitutively high levels of inflammation in their skeletal muscle or from immune cells acting on the skeletal muscle. (See, e.g. Nagaraju, K., Plotz, P., 2000, Proceedings of the National Academy of Sciences of the United States of America, 97:9209-9214.)

There is the potential that differences in the leader sequences could elicit an immune response I expressed in MHCI presenting cells. Although, for J Chain the leader sequence (seq ID No. 27) in consistent across the human population. Additionally for the immunoglobulin heavy chain the predominate leader sequence found in the human population is MDWTWRILFLVAAATGAHS (seq ID No. 28). For the immunoglobulin light chain the Kappa light chain has high sequence similarity across most if not all of the human population where the small differences in the sequences are insufficient to elicit an immune response for this reason alone. Kappa light chain leader sequences are less likely to elicit an immune response that lambda light chain leader sequences. Kappa light chain is expressed in 60% of B-cells making it the predominate light chain expressed.

Selecting for random recombination of V_{H} and V_{L} regions or Fab regions of immunoglobulin coding DNA (cDNA) through the generation of combinatorial libraries and the use of phage display technology.

In cases where isolated B-cells does not express immunoglobulins with “therapeutic binding affinity” that is may be required for broad neutralization or mitigation of the effect of allergens it may be necessary to randomly recombine V_{H} and V_{L} regions expressed by different B-cells and also to potentially use mutagenesis to further increase binding affinity. This instant patent contemplates the design of vectors of dIgA1 and dIgA2 from libraries that selected for high affinity pairs of randomly and even purposefully recombined V_{H} and V_{L} immunoglobulin regions from different B-cells and even developed in other animals such as transgenic mice or mice or other animals with humanized immune systems. One of the basic principles is to use single chain variable fragments (scFv) in phage display technology to select for highly potent immunoglobulins. One may assemble large libraries of plasmids that code for the random recombination of V_{H} and V_{L} immunoglobulin regions from different B-cells to select for high avidity and high affinity immunoglobulins where V_{H} and V_{L} regions are linked together with a short flexible peptide linker between the VH and VL fragments may occur by either N-terminus—V_{H}-linker-V_{L}—C-terminus or N-terminus—V_{L}-linker-V_{H}—C-terminus where both orientations have been applied but where N-terminus—V_{H}-linker-V_{L}—C-terminus is more common. This linked protein may be linked by a short peptide chain to the N-terminal end of a protein that is displayed on the surface of a filamentous phage for example.

Combinatorial libraries may be assembled in Escherichia coli as one typical example where E. coli contains a plasmid containing the gene encoding for the V_{H} gene and antibiotic resistance from one type of antibiotic may be transfected with a phagemid containing the V_{L} gene where both plasmids may be cleaved at a restriction site resulting in cleaved DNA where sticky ends undergo homologous recombination between the two cleaved plasmids to generate one larger circular plasmid combined that can result in productive expression of a phage that may be positively selected with the co-expression of a gene coding for antibiotic resistance from a different antibiotic that requires the other part of an antibiotic resistant gene found on the phagemid. One restriction site would be located in the region of the gene encoding for the linker that would be expressed between the V_{H} and V_{L} genes or V_{L} and V_{H} genes depending on the intended relative locations of V_{H} and V_{L} to that of the phage cell surface fusion protein. The resulting phagemid expressed a randomly recombined V_{H} and V_{L} gene that is now expressed as a fusion protein to a phage cell surface protein with the configuration N-terminal—V_{H}—linker—V_{L}—linker—phage cell surface protein—C terminal.

Phage expressing single chain variable-fragments (scFv) with high binding affinity to the antigen may be selected for by coating the antigen to a plate which can be accomplished with biotinylation and then submerging the plate in a solution containing the E. coli and phage where following washing the plate with eluting solutions the most strongly binding phage that are not washed away by elution may be identified and have their DNA encoding for the scFv amplified via PCR and sequenced. Such phage may also be used to re-infect E. coli for undergo amplification which being subjected to random mutagenesis of the V_{H} or V_{L} coding regions to create more diversity in the scFv and potentially most strongly binding phage. Random mutagenesis typically occurs at fixed regions along the antibody sequence normally targeted at the complementary-determining regions (CDRs). Site-specific mutagenesis is also employed generally after multiple cycles of random mutagenesis and phage selection based on binding affinity to the antigen biotinylated to the plate. Site-specific mutagenesis selects a defined locus of the V_{H} and/or V_{L} genes or even very specific DNA sequences. Enzyme bases mutagenesis is one form of site specific mutagenesis where a restriction endonuclease cleaves the DNA at a very specific region where oligonucleotide-mediated mutagenesis is widely employed to assist with site-specific mutation by providing internal mismatches that direct point mutations or multiple mutations to the target DNA sequence. Saturation mutagenesis employs the evaluation of substitution of a given residue against the 19 other amino acids at a specific residue.

### HIV Immunization Approach Via Antibody Gene Therapy Cocktail

This patent contemplates an immunization strategy to HIV and potentially a treatment for those infected with HIV. HIV continues to be a top priority to date and no vaccine strategy has induced antibodies with sufficient neutralizing coverage of the quasi-species. Once the host is infected with HIV control of the viral reservoir is significantly dependent on the Fc effector functions of broadly neutralizing antibodies as any loss in Fc effector activity results in the rapid loss of viral control despite potent neutralizing activity. Although, this is not necessarily true prior to infection of the host. As there are other important factors such as agglutination of HIV-1 virions in the mucosa pre infection that may be an equally important part of immune protection against HIV. To the surprise of many researchers, tissue resident natural killer (NK) cells express trivial levels of Fc receptors implying that they are unlikely to contribute to immunoglobulin mediated protection at the site of infection. It was previously reported that FcγRII and FcγRIII expressing macrophages and neutrophils were present in tissues collected from both HIV-seronegative and -seropositive subjects. Additionally, tissue-resident neutrophils while less abundant, mediated more effective phagocytic clearance of immune complexes. It has been suggested that antibody-driven functional activities mediated by cells other than NK cells are more likely to afford protection from infection as well as have therapeutic activity within mucosal and lymphoid tissues. (See e.g. Sips, M., et. al., 2016 Mucosal Immunol., 9:1584-1595.)

Human immunodeficiency virus type-1 (HIV-1) enters the host in virtually all infections through the mucosa of the genital tract or gastrointestinal tract. Once HIV traverses the epithelium in the reproductive tract HIV-1 then may encounter potential target cells such as CD4+ bearing lymphocytes including macrophages, monocytes and CD4+ helper T-cells in the lamina propria. However, it is also possible for HIV-1 to infect CD4+ bearing macrophages, monocytes and helper T-cells in the mucosa of the reproductive tract. In Memory CD4+ T cells and macrophages and monocytes of the myeloid lineage are believed to be an important reservoir for HIV-1. (See e.g. Kruize, Z., 2019, Frontiers in Microbiology, 10:1-17) Although, relatively little is known about HIV-1 infecting macrophages and monocytes in the mucosa as a result of the difficulty associated with isolating macrophages from mucosal tissue. Reproductive tract mucosal macrophages but not intestinal mucosal macrophages support the replication of the R5 HIV-1 strain. Although, intestinal macrophages have been reported to express no detectable, or very low levels of, innate response receptors and HIV-1 receptor/coreceptors and were determined to not support HIV-1 replication. However, these results are not definitive with regard to HIV-1 to infect intestinal tissue macrophages. (See E.g. Shen, R., et. al., 2009. Journal of virology, 83:3258-3267)

One mechanism by which HIV-1 infects CD4+ bearing cells by binding with its HIV-1 envelope glycoproteins which is a trimer of glycoproteins120 (gp120) and gp41. There is a conserved CD4-binding site on the HIV-1 envelope glycoprotein. Additionally, there is a conserved crest on the V3 loop that is necessary for chemokine receptor type 5 (CCR5) or chemokine receptor type 4 (CXCR-4) co-receptor binding that is a key event for HIV envelope fusion with the CD4 bearing T-cell, macrophage or monocyte. Additionally, HIV-1 endocytosis by a micropinocytosis-like mechanism can lead to productive infection in macrophages. In order for micropinocytosis to occur in HIV-1 endocytosis the CCR5 receptor is thought to be present in the engulfed endosome. This pathway specifically requires CCR5 engagement at the cell surface, which in turn suggests that the virus and its coreceptor are present in the endosomal environment simultaneously since the principle of fusion with the endosome membrane is no different than the principle of viral fusion of HIV with an endosome. While HIV undergoes efficient viral degradation following endocytosis, analyses haves supported that HIV-1 transport through the endolysosomal pathway occurs throg in delayed viral degradation following endosomal internalization, possibly allowing the virus to complete its fusion. (See e.g. Gobeil, et. al., 2013, J Virol. 87:735-45, 2013; Carter, G. C., Bernstone, L., Baskaran, D., James, W., 2011, Virology, 409:234-250) This pathway of viral fusion during or after endocytosis is a critical pathway for HIV-1 that must be blocked by antibodies in the mucosal environment for any immunization to be effective. Although, it is generally thought that endocytosis represents a dead end for HIV infection with the caveat of this viral fusion pathway that is thought to require CCR5 or CXCR4 binding to the HIV envelope glycoprotein which occurs at the conserved crest of the V3 loop. In one embodiment a dIgA1 antibody is considered for the conserved crest of the V3 loop is considered.

Although, there are additional sites on the HIV envelope glycoprotein that could be targeted as part of a cocktail strategy to maximize efficacy. Important clues to such a strategy would target conserved regions of the HIV envelop glycoprotein two such conserve regions are the Variable Region 1-Variable Region 2 (V1/V2) regions and the CD4 binding site. A recent HIV-1 clinical trial in Thailand known as RV144 resulted in 31.2% efficacy. (See Tomaras, G. D., et. al., 2013, Proceedings of the National Academy of Sciences of the United States of America, 110:9019-9024.) In this clinical trial the authors reported that “Env-specific plasma IgA/IgG ratios are higher in infected than in uninfected vaccine recipients in RV144.” The report also discussed “Though plasma Env variable region 1 and 2 (V1/V2) IgG correlated with decreased infection risk, high levels of anti-HIV-1 Env plasma IgA correlated with increased infection risk.” That authors attributed this to the difference in effector functions between IgG and IgA. IgG1 for example has FcγRIIIa binding sites for macrophages and neutrophils that IgA does not have where IgA may be less effective at eliciting a pro-inflammatory response from macrophages and neutrophils in the mucosa and also less effective at recruiting natural killer (NK) cells in the bloodstream. The authors reference the following report that stated in the report “the binding of IgG antibodies to variable regions 1 and 2 (V1V2) of HIV-1 envelope proteins (Env) correlated inversely with the rate of HIV-1 infection (estimated odds ratio, 0.57 per 1-SD increase; P=0.02; q=0.08), and the binding of plasma IgA antibodies to Env correlated directly with the rate of infection” (Haynes B F, et al., 2012, N Engl J Med vol. 366:1275-1286). It may be beneficial to incorporate an IgG antibody into an HIV Immunization gene therapy cocktail specific to the V1/V2 regions while having no competing IgA or dIgA specific to the IgG binding site. This is only possible with direct administration of antibodies or a gene therapy based approach. As using a traditional vaccination approach of developing immunity in one immune system from exposure of virus antigens cannot guarantee the ratios of IgG to IgA nor can it guarantee the specificity of the antibodies and their ability to be broadly neutralizing.

The third important binding site is the elusive CD4+ binding site that is typically responsible for HIV's initial binding to the CD4 presenting immune cell. Where a third potential gene therapy antibody cocktail immunoglobulin could be a IgG1 or dIgA1 specific to the HIV CD4 binding site. Such an antibody would block the initiation event of HIV binding to immune cells that precedes the binding to the CCR5 or CXCR-4 binding sites. Although, it is recognized that such binding with the CD4 receptor may not be necessary during HIV-1 endocytosis by a micropinocytosis-like mechanism suggesting that targeting the crest of the V3 loop with dIgA1 is of chief importance and may be sufficient as a stand-alone approach.

In one embodiment an integration competent lentiviral vector or an integration-deficient lentiviral vector encoding for a dIgA1 antibody specific to a conserved target and receptor binding site on the HIV envelope glycoprotein is delivered via a anti CD20+ pseudotyped lentiviral delivery system to B-cells that bears a CD20+ receptor that include naïve B-cells, memory B-cells and germinal center memory B-cells. The vector will be integrated into genomic DNA or be delivered as episomal DNA and will code for a strong promoter that results in a higher level of expression of the vector encoded dIgA1 that the naturally encoded immunoglobulin encoded for by the B-cell resulting in the vector encoded dIgA1 being the major product. After some period following lentivirus administration an mRNA vaccine encoding for the HIV envelop glycoprotein is administered and even a second mRNA booster will be administered some period following. This, will have the effect of activating a large number of B-cells to become memory plasma B-cells potentially persisting for decades to express the dIgA1 specific to the conserved sequence of the envelope glycoprotein. In additional the administration of the mRNA B-cell activator will also result in the development of T follicular helper cells to active Germinal Center B-cells that contain the lentiviral vector.

H. pylori and Other Bacteria

This patent contemplates a strategy to neutralize a variety of bacteria. SIgA is known to neutralize bacterial by binding and agglutinating them preventing the formation of colonies and also binding to their flagella preventing their motility. SIgA also neutralizes bacterial products such as enzymes and toxins. This patent contemplates a strategy to encode for dIgA2 specific to the gram-negative, spiral shaped and microaerophilic bacteria Helicobacter pylori (H. pylori). H. pylori is a leading cause of gastric cancer death worldwide possibly causing over 700,000 deaths yearly and about 15,000 deaths in the U.S. yearly. Additionally, H. pylori is the only bacteria classified by the WHO as a class I carcinogen meaning it is the only bacteria well established to cause cancer. Most bacterial show tropism to specific tissues or cell types and often can use many different adherence mechanisms for attachment. Some speculate, that H. pylori may use as many as 5 different adhesins to attach to gastric epithelial cells. H. pylori has extensive gene variation within its genome that is thought to occur through slipped-strand mispairing within repeats as mechanisms for antigenic variation and adaptive evolution. H. pylori features a genome with high plasticity and high genetic heterogeneity especially in some parts of outer membrane proteins making them difficult targets. Although, there is some sequence identity is observed across some of the H. pylori outer membrane proteins that are thought to be necessary for adherence to the gastric epithelium. A common attachment site on epithelial cells is the Lewis histo-blood group antigens. H. pylori has a large number of sequence-related genes that are expressed on its outer surface. While the exact mechanism related to how H. pylori causes cancer is under investigation there are generally a few requirements for H. pylori to be cancer causing. H. pylori must be able to bind to the gastric epithelium and colonize in addition to burrowing through the epithelium causing stomach or small intestine ulcers in order to cause cancer. Additionally, 50% of H pylori induce vacuolation of epithelial cells that may lead to cancer through the action of vacuolating cytotoxin A (VacA) that is thought to induce apoptosis of epithelial cells that results in vacuoles that have traits of both endosomes and lysosomes. (See e.g., Atherton, J. C., Blaser, M. J., 1995, Cover, T. L., J Biol Chem, Jvol. 270:17771-17777; Kuck, D., Rudi, J., et al. 2001, Infection and immunity, 69:5080-5087) Although, while VacA induces apoptosis in epithelial cells it also disrupts endolysosomal vesicular trafficking and impairs the autophagy pathway. This makes it more difficult for the epithelial cell that has underwent apoptosis to be cleared away and replaced by new epithelial cells. Additionally, the virulence factor cytotoxin associated gene A (CagA) is a known oncoprotein that contributes to the development of gastric cancer. CagA has been shown to be dependent on VacA. It has been shown that in the absence of VacA two different cellular mechanisms proteosome degradation and autophagy degrade CagA. (See e.g., Abdullah, M., Bronte-Tinkew, D. et al., 2019, Sci Rep, vol. 9, pp. 38.; Palframan, S. L., Gabriel, K., 2012, Frontiers in Cellular and Infection Microbiology, 2:1-9) Overall, results suggests that H. pylori may cause stomach cancer through more than one mechanism. Thus, targeting the cancer causing mechanism may not be an effective stand-alone means to mitigate the effects of H. pylori. What is equally important is that H. pylori does not act virulent unless it colonizes. That is the Beta Barrel opening resulting from VacA and resulting cleavage of the virulent peptide does not occur unless H. pylori is adhered to the epithelial cell and colonized and H. pylori does not secrete CagA and other toxins through a membrane channel unless it is colonized. These facts make VacA and CagA difficult targets because they are less likely to present on the outer membrane unless H. pylori is virulent. In order to colonize H. pylori must effectively adhere to epithelial cells. Attachment is mediated through cell surface adhesins. This results in a restricted range of hosts and tissues utilized for colonization. If bacteria are unable to adhere to epithelial cells they tend to be rapidly removed by the shedding of the surface cells and mucus layer. (See, e.g. Borén T, Falk P, Roth K A, Larson G, Normark S., 1993, Science., 262:1892-5) Thus, the most effective preventative measure against H. pylori infection would ideally involve targeting at least one protein that is important in binding to epithelial cells that has a well-conserved sequence on the surface that can be targeted.

The most ideal protein to target would ideally be common among H. pylori strains with a conserved sequence on the outer surface. HopQ I has been reported to be represented in 72.5% of H. pylori strains. HopQ is a porin that facilitates transfer of the CagA. HopQ can exploit the carcinoembryonic antigen-related cell adhesion molecule family (CEACAMs). HopQ binds to the IgV-like domain at the N-terminal of CEACAMs to facilitate the transfer of crucial pathogenic factor CagA to host cells. The HopQ-CEACAM interaction has been measured to be of high affinity (K_{D} from 23 to 268 nM), independent of CEACAM glycosylation. Data has supports that the HopQ-CEACAM interaction contributes to gastric colonization or Hp-induced pathologies. (Koniger, V., Sundberg, E. J., et. al., 2018, The EMBO journal, 37:e98664; Xu, C., Soyfoo, D. M., Wu, Y., & Xu, S., 2020, European journal of clinical microbiology & infectious diseases: official publication of the European Society of Clinical Microbiology, 39:1821-1830) Use of dIgA2 which becomes SIgA2 for this purpose satisfies two requirements first key binding sites can be blocked on H. pylori reducing its ability to bind to epithelial cells. Second, dIgA2 can effectively agglutinate H. pylori facilitating its passage through the digestive system followed by its excretion. For example, it is that cell specific attachment of H. pylori to human gastric surface mucous cells is inhibited by human colostrum secretory immunoglobulin A (SIgA) which is a glycoprotein carrying a highly variable set of N- and O-linked oligosaccharides. The inhibitory activity of SIgA is reduced when it is deglycosylated by digestion with alpha-L-fucosidase. (See, e.g. Boren T, Falk P, Normark S., et al., 1993, Science., 262:1892-5).

This patent contemplates that administration of H. pylori outer membrane proteins and secreted proteins as well as their variants as a vaccine delivered to the lamina propria of the stomach and small intestine. In one embodiment the proteins are administered directly and in another embodiment mRNAs encoding for the proteins are administered via lipid nano particles or other vesicle based deliver vehicles. This patent further contemplates the use of adjuvants administered with the H. pylori proteins to enhance the immune response and the development of memory B-cells sensitive to H. pylori proteins.

### Biodistribution of Antibody in Interstitial Tissues Underlying the Mucosal Epithelium

This instant patent contemplates the major advantage of dIgA1 and dIgA2 over other immunoglobulins and achieving therapeutically relevant levels of dIgA1 and dIgA2 expression in the regions where they are needed to exert their therapeutic benefit. To achieve therapeutically relevant levels of SIgA1 and SigA2 in the mucosa the underlying interstitium of the lamina propria must reach sufficient concentrations of the therapeutically relevant dIgA1 or dIgA2. Lymph nodes typically align the areas directly outside the smooth muscle tissue (muscularis) of the lamina propria. These lymph nodes supply the interstitium of the lamina propria with plasma B-cells, memory plasma B-cells (that may ultimately migrated to the bone marrow) and some memory B-cells that are specialized in that most of them produce dIgA1 and dIgA2 as opposed to monomeric immunoglobulins more typically found for B-cell in the circulating blood. Thus, typically, dIgA antibodies are supplied right at the site where they undergo transcytosis upon binding with pIgR and the interstitium which is between 1 and 2 mm wide. That is the distance between the smooth muscle and the basal face of the epithelium is the interstitium. Thus, if dIgA1 or dIgA2 is supplied from muscle cells, liver cells or circulating memory B-cells or memory B-cell in the lymph nodes support the lamina propria they must be supplied through the blood stream where their concentration in the lamina propria will be less than the concentration in the blood. Alternatively, they may be ultimately supplied by memory plasma B-cells derived from memory B-cells in the circulating blood or Germinal center memory B-cells that received integration competent or integration-deficient lentiviral vectors. Antibody Levels in organs and interstitial tissues such as those that underlie the mucosal epithelium in the digestive tract, reproductive tract, respiratory tract, kidneys and the skin have been evaluated for Immunoglobulin class G monoclonal antibodies. In the stomach and small intestine the IgG monoclonal antibody concentration in the interstitium of the lamina propria is 2-4% of that of the concentration of the circulating blood after subtracting out the residual plasma in the interstitial vasculature. In the lungs the IgG monoclonal antibody concentration is about 7% of that of the circulating blood after subtracting out the residual plasma in the interstitial vasculature. (See E.g. Eigenmann, M. J., Karlsen, T. V., Krippendorff, B. F., et. al., 2017, J Physiol.; 595:7311-7330) In the skin the IgG monoclonal antibody concentration is about 10% of that of the circulating blood. The unidirectional transport of dIgA1 and dIgA2 from the lamina propria to the mucosa will lead to an overall higher concentration in the lung, digestive and parts of the reproductive tracts in the likely in area of a 50% or greater increase over that of IgG up to some threshold concentration that will unquestionably be therapeutically significant since we must rely on these systems throughout our lives to ensure mucosal protection. Thus, lung lamina propria and mucosa dIgA levels will reach a dIgA concentration in the area of 11% of that of the circulating blood level concentration of dIgA. In the stomach and small intestine circulating antibodies can pass freely through the capillary endothelium in the interstitium that has fenestrations of 60-80 nm allowing for fast equilibration and replacement of any dIgA that is transported to the mucosa. dIgA is 22-26 nm in length. The digestive tract lamina propria and mucosa are likely to see even higher concentration increases in dIgA over that reported for IgG due to the rapid equilibration that occurs between the fenestrated capillary endothelium and the lamina propria thus dIgA concentrations of 4-7% of the circulating blood a level are possible. Although, the capillary endothelium of the lung interstitium are continuous and lack fenestrations. In the kidneys the interstitial concentrations are

Because dIgA is actively and unidirectionally transcytosed across the epithelium with pIgR the dIgA mucosa to blood distribution can be enhanced favorably despite low interstitial concentrations relative to dIgA blood levels. In one embodiment dIgA is supplied from hepatocytes, in another embodiment dIgA is supplied by muscle cells. In another embodiment dIgA is supplied by spleen cells that receive and express the gene therapy vectors. (See E.g. Borrok, M. J., DiGiandomenico, A., Beyaz, N., Marchetti, G. M., Barnes, A. S., Lekstrom, K. J., Phipps, S. S., McCarthy, M. P., Wu, H., Dall'Acqua, W. F., Tsui, P., & Gupta, R., 2018, JCI insight, 3:97844:1-9).

### The Interstitium of the Lamina Propria

This patent contemplates direct administration of the gene therapy via endoscopic injection or absorption into the lamina propria. The presence of SIgA in mucosal surfaces is locally produced as dIgA by antibody secreting B-cells and memory plasma B-cells in the lamina propria directly beneath the epithelial cells. The source of these dIgA B-cells is the local lymph nodes for the organ. As an example, the stomach has 4 supporting groups of lymph nodes support 4 distinct regions of the stomach. The small intestine also has a supporting lymph nodes in addition to Payer's patches that are sometimes located in the upper duodenum but more often found in the mid to lower duodenum and in other regions of the small intestine. Nutrient and fluid absorption in the Gastrointestinal tract requires lymphatic networks to both regulate interstitial fluid balance and transport lipids. Lymphatic flow in the stomach begins within the initial lymphatic sinuses near the pyloric glands. These fuse to link lymphatic networks between the muscularis mucosa and the submucosa. (See, e.g., Spencer, J., Sollid, L., 2016, The human intestinal B-cell response. Mucosal Immunol 9, 1113-1124.; Lycke, N., Bemark, M., 2017, Mucosal Immunol, 10:1361-1374; Eichmann, A., 2019, Cellular and Molecular Gastroenterology and Hepatology, 7:503-513)

The embodiment of the invention related to gene therapy delivery to memory B-cells in lymph nodes supporting an organ with a mucosa is achieved through the initial lymphatics in the lamina propria which contain button like openings that allow for flow from the fluid filled interstitum into the lymph vessels into the afferent lymphatic vessels that carry unfiltered lymph fluid passing through local lymph nodes to be filtered. Such initial lymphatics are found in the lungs and GI tract. At the initial lymphatics there are distinct leaf shaped endothelial cells that are the general locations of fluid entry into the lymphatic system. The borders of these epithelial cells have been thought to be the primary valves that permit unidirectional flow of fluid into lymphatics. In the trachea and thought to be the case for the stomach and small intestine the borders of these epithelial cells are thought to contain discontinuous tight junctions made by VE-cadherin that is consistent with their function as connection points along the sides of flaps that are effectively the borders for the button like openings for fluid passage without junctional disassembly. Fluid entry into the button like openings is driven by muscle contraction. The buttons can be thought of as linear segments that are parallel to each other with openings of about 3.2 μm (micrometers) or 3,200 nm (nanometers) that were spaced about 2.9 μm apart in the trachea. (See, e.g., Baluk, P., et. al., The Journal of experimental medicine, vol. 204, pp. 2349-2362, 2007; Eichmann, A., 2019, Cellular and Molecular Gastroenterology and Hepatology, 7:503-513)

When a lymphangion, the functional unit of a lymph vessel, has contractile force exerted on it e.g. as due to muscle contraction of smooth muscle linking lymph vessels lymph can be propelled forward in a unidirectional manner. The semilunar valves are directed towards the flow of the lymph and open when the pressure in the first lymphangion is greater than the pressure in the next lymphangion. Pressure in the first lymphangion may increase because of smooth muscle contraction (in lymph vessel) or because of pressure on the walls from outside. Once the lymph flows into the next lymphangion, it cannot return to the previous lymphangion, as the semilunar valves close tightly. (See, e.g. Venugopal, A. M., Stewart, R. H., Laine, G. A., Dongaonkar, R. M., Quick, C. M., 2007, Am J Physiol Heart Circ., 293: H1183-9)

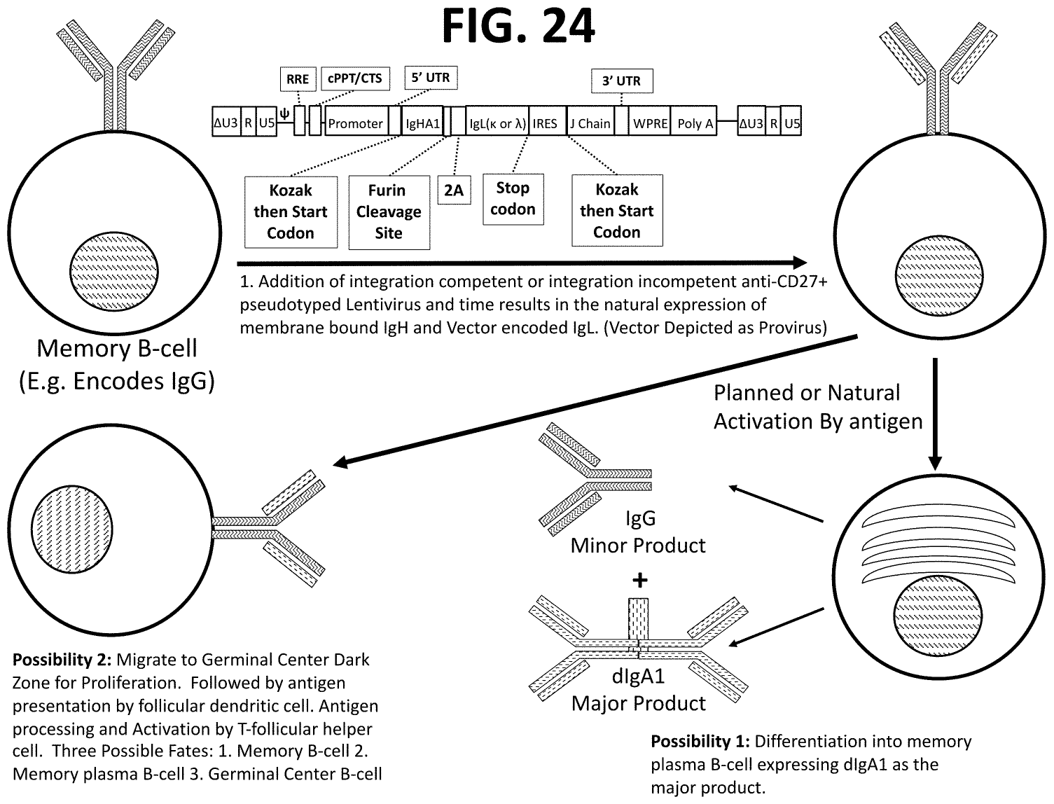

In the stomach and small intestine, lymphatic capillaries, or lacteals, are located exclusively in intestinal villi. Spontaneous lacteal contraction, in concert with adjacent smooth muscles, is essential for drainage into lymph nodes. Lacteal contraction is regulated by the autonomic nervous system, and to be increased by acetylcholine and decreased by norepinephrine. Additionally, such drainage can be accomplished with massaging the stomach in humans. The lacteals lead to lymph nodes supporting the organ. (See e.g., Cifarelli, V., Eichmann, A., 2019, Cellular and Molecular Gastroenterology and Hepatology, 7:503-513) In one embodiment those lymph nodes that house germinal center memory B-cells will receive the gene therapy delivery vehicle such as a Lentivirus that is known to be absorbed by memory B-cells via a pseudotyped lentivirus that can target the CD receptors such as CD19, CD20 or CD27 on the memory B-cell (See FIG. 24) (See, e.g. Cascalho, M., et al., 2018, Sci Rep, 8:11143; Also see, Fusil, F., Cosset, F. L. et. al., 2015, Molecular therapy: the journal of the American Society of Gene Therapy, 23:1734-1747) Memory B-cells and complete retroviral insertion of the gene therapy expression dIgA1 into the genomic DNA of the B-cell where all subsequent plasma secreting B-cells and Germinal Center memory B-cells derived from such B-cells would produce the vector encoded dIgA antibody specific to the target of interest at a much higher level than the naturally encoded immunoglobulin. Upon activation the memory B-cell would give rise to memory plasma B-cells that will migrate to the tissues supported by the lymph node they are derived from e.g. the lungs or stomach and upper duodenum via local blood flow where they would secrete the dIgA encoded for by the vector delivered gene therapy delivery vehicle as the major immunoglobulin product through the use of a strong promoter. In another embodiment CD45+ Plasma B-cells in the Lamina Propria will also be targeted by the integration competent or integration deficient lentivirus. (See e.g., Spencer, J., Sollid, L., 2016, The human intestinal B-cell response. Mucosal Immunol 9, 1113-1124). It was previously demonstrated that a pseudotyped lentiviral vector could target B-cells retrovirally incorporate the retroviral vector and conditionally express both the membrane anchored and secreted forms of antibodies encoded for by the vector as the major immunoglobulin product with the use of a strong promoter and through alternative splicing that was dependent on the B-cell maturation state. (See, e.g., Fusil, F., Cosset, F. L. et. al., 2015, Molecular therapy: The Journal of the American Society of Gene Therapy, 23:1734-1747). (Also see the International patent filed by the authors WO 2017/005923 expressly incorporated by reference herein in its entirety) The authors of this report focused on the expression of IgG1 and considered it as a strategy for both HIV and cancer.

This patent further contemplates the use of proteins or mRNA encoding for proteins delivered to the lamina propria of the at risk organ identifiable as or derived from the (1) virus (s) (2) systemic ailment (3) fungi (4) bacterial infection (4) cancerous tumor (5) biowarfare agent (6) microbial infection, (7) any ailment (8) target protein or variant of interest. This patent targets the lamina propria and underlying interstitium as a means to reach the lymph nodes supporting the organ of interest. In one embodiment the gastrointestinal tract such as stomach and small intestine is targeted. In another embodiment the lungs are targeted. The lymph nodes would be reached by the vaccine traveling though the underlying lymph vessels in the underlying organ.

There is currently an FDA approved endoscopic injection therapy dextranomer/hyaluronic acid copolymer (Deflux) for the treatment of vesicoureteral reflux. The endoscopic injection treatment associated with this approach is considered minimally invasive, performed on an outpatient basis, and technically straightforward, with a relatively short learning curve and low complication rate. These advantages have led to its widespread use in last 2 decades. In extrapolating from this endoscopy based injection to the lamina propria there is a clear precedent that could be extrapolated to a similar procedure for administering the dIgA1 or dIgA2 gene therapy on one or more of the stomach, upper duodenum, esophagus, the trachea and bronchi.

### 3′ Untranslated Regions (3′ UTR) and 5′ Untranslated Regions (5′ UTR)

Important elements of RNA are the 5′ UTR that is upstream of the beneficial gene and the 3′ UTR that is downstream of the beneficial gene. UTRs serve a variety of roles including enhancing the amount of protein that is produced from a single RNA, efficient transport outside the nucleus, stability of the mRNA increasing the mRNA half-life and the ability to control the rate of gene translation.

The 5′ UTR is also known as the leader sequence (not to be confused with the immunoglobulin leader sequence or signal peptide) and is the region of mRNA directly upstream of the initiation codon. 5′ UTRs can form complex secondary structures to regulate translation on two levels by preventing translation or as a tertiary structure necessary for binding and recognition by translation factors. Regulatory elements in the 5′ UTR are also linked to mRNA export outside the nucleus. 5′ UTRs that allow for efficient translation are short, have a low GC content, do not contain upstream AUG codons and are relatively unstructured. A major aim of gene therapy is to minimize the dosage necessary to achieve a therapeutically relevant amount of the protein that is being encoded for which this instant patent contemplates to ensure high expression of vector encoded immunoglobulin in this instant patent. In general it has been shown that shorter 5′ UTR sequences are responsible for higher levels of protein expression when the same 3′ UTR is used to express the same protein. (See e.g., Trepotec, Z., et. al., 2019, Tissue Eng Part A., 1-2:69-79; Leppek, K., Das, R. & Barna, M., 2018, Nat Rev Mol Cell Biol, 19:158-174.; Calvo, S. E., Pagliarini, D. J., et. al., 2009, PNAS, 18:7507-7512; Babendure, J. R., et. al., 2006, RNA, 12:851-861.; Barrett, L. W., et. al., 2012, Cellular and molecular life sciences: CMLS, 69:3613-3634)

Within eukaryotic genes the 5′ UTR is located downstream of the TATA box. The TATA box is the transcription initiation site. Although, there is spacer DNA between the 5′ UTR and the TATA box that is typically about 20 bases. This spacer DNA is necessary for the transcriptional machinery to assemble and begin transcription about 20 bases downstream from the TATA box. For example, TATA box binding protein binding the TATA box with other cofactors followed by RNA polymerase which pushes the transcription start site downstream from the TATA box.

5′ UTRs for proteins intended for high expression typically are very short in the area of 20 to 30 bases including the (−1 through −6 position on the Kozak consensus sequence). For example the protein J chain that is expressed in a dIgA plasma B-cell and in a dIgA memory plasma B-cell at a rate high enough to support 500 dIgA1 immunoglobulins per second (requiring 250 J Chains per second) is has a 5′ UTR of 18 bases including the Kozak consensus sequence. (For a list of 5′ UTRs found in immunoglobulins and selected highly expressed genes see Table 2)

### 3′ UTR

The 3′ UTR is located downstream of the protein coding sequence is involved in numerous regulatory processes including transcript cleavage, stability, translation and mRNA localization. 3′ UTRs effectively determine the fate of mRNA. Sequence constrains are more relaxed in the 3′ UTR allowing for a higher degree of regulation. Although, there are regions of high conservation within the mammalian genome. (Barrett, L. W., et. al., 2012, Cellular and molecular life sciences: CMLS, 69:3613-3634; Siepel, A., et. al., 2005, Genome research, 15:1034-1050). In contrary to promoter regions motifs within the 3′ UTR are conserved on one strand as regulatory proteins regulate translation and even localization by engaging with the conserved motifs. In addition, microRNA binding sites are found in the 3′ UTR that allow miRNAs to decrease the level of expression of the mRNA by the ribosome, blocking regulatory proteins from binding and marking the mRNA for degradation. In addition there are silencer regions within the 3′ UTR that repressor proteins may bind to and inhibit expression.

Recently, it was shown that in human cell lines that 3′ UTRs allow for differentially regulated localization of some proteins to membranes. As an example, the long 3′UTR of the mRNA of CD47 enables efficient transport to the cell surface where it is localized on the surface on the cell. Alternatively the short 3′ UTR of the mRNA of CD47 localizes to the endoplasmic reticulum. The authors of this study concluded that the long 3′UTRs contain additional regulatory elements that can regulate localization and protein abundance. Although, the localization step occurs at the protein level where the mRNA with both the long 3′ UTRs and short 3′ UTRs of CD47 have a similar distribution at the perinuclear endoplasmic reticulum. Thus, the localization of the protein is independent of the localization of mRNA. (See, e.g. Berkovits, B., Mayr, C., 2015, Nature 522:363-367). Immunoglobulins that are expressed on the cell surface likewise have a 3′ UTR that is much longer than the 3′ UTR of the same class and subclass of immunoglobulins that are expressed for secretion. Although, both these immunoglobulins must ultimately reach the surface of the cell. Although, through different pathways. (For a list of 3′ UTRs found in immunoglobulins and selected highly expressed genes see Table 3)

B-Cell Differentiation into Plasma Secreting Cells

The differentiation of B cells into Ig-secreting plasma cells (both memory plasma B-cells and short lived plasma B-cells) requires the expansion of secretory organelles to cope with the increased cargo load. When any B-cells differentiates into a short-lived plasma B-cell or a memory plasma B-cell the cell increases in size significantly while the endoplasmic reticulum (ER) continues to remain close to the nucleus of the cell. Later during interphase the ER fills the entire cell for efficient secretion of immunoglobulins via the extension of ER tubules under the plasma membrane. At the same time the Golgi remains near the nucleus while expanding to 6.5 fold in linear volume mostly linearly while remaining close to the nucleus. Immunoglobulin secretion rate increases dramatically by day 4 following B-cell activation as by day 3 ER proliferation is quite significant and can more readily support high expression rates of immunoglobulins. From this it is evident that without an unusually relatively large dedication to the ER and Golgi plasma secreting B-cells would secrete immunoglobulins at a much lower rate as was empirically observed. (See e.g. Kirk, S. J., Cliff, J. M., Thomas, J. A., Ward, T. H., 2010, J Leukoc Biol. 87:245-255).

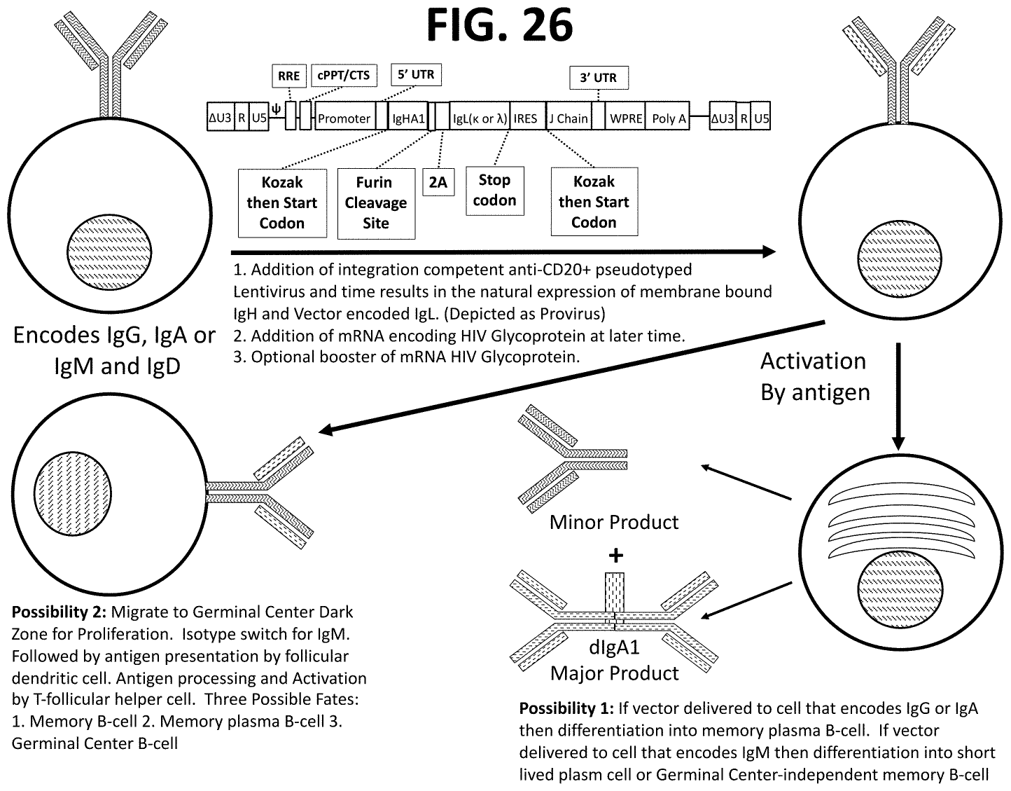

This patent contemplates the delivery of gene therapy vectors encoding for immunoglobulins to cells with a large dedication to production of proteins and secretion of proteins as evident by extensive resources dedicated to the ER and Golgi in addition to a large ER and Golgi. For example liver cells or hepatocytes secrete a large amount of albumin and fibrinogen, alpha-1-globulin, alpha-2-globulin and beta globulin. (See e.g. Feldmann, G., Penaud-Laurencin, J., Crassous, J., Benhamou, J. P., 1972, Gastroenterology, 63:1036-1049; Woo, D. H., et. al., 2012, Gastroenterology. 142:602-11; Sharma, N. S., Nagrath, D., & Yarmush, M. L., 2011, PloS one, 6:e20137) Muscle cells must produce large amounts of actin and myosin necessary to support the muscular-skeletal structure. For this reason muscle cells and hepatocytes are able to produce and immunoglobulins at therapeutically relevant levels. (See e.g. Lei, Y., Huang, T., Su, M. et al., 2014, Lab Invest 94:1283-1295; also see Perez, N., et. al., 2005, Genetic vaccines and therapy, 2:1-5) For a similar reason, memory B-cells are also well suited for genomic integration of lentiviral DNA or episomal addition of lentiviral DNA coding for immunoglobulins as upon their differentiation into memory plasma B-cells will have ample resources dedicated to producing and secreting antibodies. (See, e.g. Cascalho, M., et al., 2018, Sci Rep, 8:11143) This patent contemplates targeting memory B-cells for integration-competent lenviral vectors and integration deficient lentiviral vectors encoding for dIgA. In one embodiments (See FIGS. 24 and 26) the memory B-cell will present on its surface both the endogenous heavy and light chain immunoglobulins as a B-cell receptor as well as the endogenous heavy chain in a heterodimer with the genome integrated lentiviral vector or episomally encoded integration-deficient lentiviral vector encoded light chain immunoglobulin as a B-cell receptor. Thus, having the gene therapy encoded immunoglobulin light chain presented on the memory B-cell will increase the probability that it will be activated from an H. pylori infection—or other bacterial infection or for any antigen or any protein target the B-cell surface immunoglobulin has sufficient affinity for. As the immunoglobulin light chain is ideally suited for antigen recognition as it tends to play a dominant role in target binding. Thus, the light chain binding the target will be sufficient in most cases to activate the memory B-cell to differentiate into a memory plasma B-cell or a germinal center B-cell. (See, e.g. Sun, M., Li, L., Sheng Gao, Q., Pad S., 1994, The Journal of Biological Chemistry, 269:734-738; Also see, Hadzidimitriou, A., Darzentas, N., Belessi, C., et. al., 2009, Blood, 113:403-411).

In some instances plasma B-cells and memory plasma B-cells may code for truncated immunoglobulin heavy or light chains through somatic hypermutations nonsense mutations and would then still produce the vector encoded dIgA and in other instances the cell will undergo apoptosis. Although, it is important to point out that even with the presence of the naturally encoded immunoglobulins by the cell by using a strong promoter in the vector encoded immunoglobulin the major product of a memory plasma B-cell with the vector encoded immunoglobulin will be the vector encoded dIgA1 immunoglobulin. In contrast to the immunoglobulin Heavy chain that has a large 3′ UTR when it is expressed as a cell surface receptor because the immunoglobulin light chain has the same 3′ UTR whether it is expressed as part of a B-cell receptor or secreted it will serve as an adequate substitute for the immunoglobulin light chain that is endogenously encoded for by the cell.

mRNA Production

mRNA intended for gene therapy may be synthesized in vitro as part of template directed synthesis. Common methods include the T #, T7 and SP6 systems. The T7 system derived from the T7 phage of E. Coli is the most common method. The sequence of interest is placed downstream of the T7 promoter which covers the sequence from −17 to +6 (the first 6 RNA nucleotides to be synthesized) Thus, the first 6 RNA nucleotides do not have full flexibility of choice in the T7 system. One such class III T7 promoter is 5′-TAATACGACTCACTATAGGGAGA-3′. Transcription termination occurs at terminator sites called Rho-independent terminators. Here the 3′ end of mRNA forms a hairpin loop structure about 7-20 base pairs in length directly following the U heavy stretch. This hairpin loop formation results in pausing of RNA polymerase and disrupts the transcription complex. Alternatively, the termination of transcription may occur by the RNA polymerase running off at the end of the template where the formation of a hairpin structure is not necessary.

### Allergens

This patent contemplates the use of dIgA1, dIgA2 and engineered variants of dIgA gene therapy to mitigate allergies. In one embodiment allergies of the respiratory tract are targeted with dIgA1. In another embodiment allergies of the gastrointestinal tract are targeted with dIgA1 or dIgA2. Type I hypersensitivity (or immediate hypersensitivity) is an allergic reaction provoked by re-exposure to a specific type of antigen referred to as an allergen. When an antigen is not associated with a pathogen or infectious agent causes hypersensitivity to those exposed to such antigens the hypersensitivity is referred to as an allergic reaction and the antigen is referred to as an allergen. Allergic reactions can be caused by allergens binding 2 adjacent Immunoglobulin class E (IgE) antibodies bound to adjacent IgE receptors (FcεRI) on mast cells.

In many allergic reactions humans become sensitized to the innocuous antigen and produce IgE antibodies against it. Future exposure to the antigen causes the activation of IgE binding cells mainly mast cells and basophils. Allergies may occur when the allergens cross the mucus membrane of the nasal cavity, eyes, lungs or skin as examples. And genetic factors are thought to play a role in allergies. For those with allergies to an allergen IgE specific to that antigen tends to be highly elevated in the interstitium lining airways and thus, the most effective combat to prevent the allergen from binding IgE would be dIgA1 that is present both in the interstitium and at the mucus barrier in its SIgA form. There are other allergies such as forms of asthma that are due to cytokines including interleukins (IL) and do not have a causal link to IgE.

Allergen induced allergies occur through contact with the mucosa of the respiratory tract, digestive tract, through skin absorption or through blood circulation in some cases such as a bee sting. In the respiratory tract common allergies include pollen, birch, dust mite feces, and cat dandruff causing asthma. About half of the U.S. population is sensitive to at least one innocuous antigen or allergen. The most common allergies in developed countries are from airborne allergens resulting in symptoms mainly affecting the nasal passage and lower airways including the lungs which is classified as asthma. In some cases people are sensitive to one specific allergen and in other cases have nonspecific sensitivity to a range of allergens. When people are sensitive to specific allergens or allergen classes strategies that employ neutralization and degradation of the antigens respectively with SIgA and dIgA may effectively be used. Allergens that are ingested such as proteins found in specific foods that result in allergies are sometimes but not always limited to the gastrointestinal tract that has its own lymphatic support system.

Mast cells have high affinity IgE receptors (FcεRI) that tightly bind to IgE and when an allergen the individual is sensitized to crosses the epithelial cells mast cells can detect them generally via two adjacent IgE bound to the FcεRI. Thus, allergens generally have to have more than one identical or nearly identical binding faces (as far as the immunoglobulin is able to detect) to result in an allergic reaction. When an antigen is bound to IgEs bound to FcεR on mast cells it results in the mast cell to release granules containing cytokines, histamine, tryptase, Platelet-activating factor that causes allergic symptoms and in individuals with asthma cause smooth muscle contraction and mucosal edema and can cause anaphylaxis. Eosinophils are also activated by Mast cells with IgE bound to antigens with IL-5 and can result in eczema and deadly anaphylaxis. There are two phases of sensitization to allergens: The induction phase and the effector phase. The induction phase involves many different cells and proteins including epithelial cells, chemokine ligand 27 (CCL27), immature dendritic cells, antigen presenting dendritic cells, TH2 T-helper cells, cytokines, such as interleukin (IL)-4, IL-5 and IL-13, class switching of B cells to IgE secreting B-cells, IgE secretion and binding to the high-affinity IgE receptor (FcεRI) on the membrane of mast cells and basophils, forming sensitized mast cells and basophils. When immature dendric cells respond via pattern recognition receptors to danger signals they mature into competent antigen-presenting myeloid-type dendritic cells. Generally, allergen detection occurs when these mature dendritic cells subsequently bind and processes the allergen presenting a polypeptide fragment of the allergen via the major histocompatibility complex class II (MHCII) receptor where the dendritic cell subsequently migrates to the local lymph node where they interact with naïve T-cells (TN) through their T-cell receptor (TCR) via MHCII and co-stimulatory molecules that results in TH2 T-helper cells maturation and migration to the local tissue such as the lungs interstitium subsequent interactions as a result of the same or sufficiently similar allergen between the MHCII of dendritic cells and the TCR of T_{H}2 T-helper cells results in the secretion of IL-4 and IL-13. IL-4 and IL-13 stimulate immature IgM or IgD B-cells bound to the allergen to class-switch into IgE B-cells and secrete IgE that can bind IgE FcεRI receptors on mast cells. (See, e.g. He, S. H., Zhang, H., Yang, P. C., 2013, Acta pharmacologica Sinica, 34:1270-1283)

The effector phase begins when the same or a sufficiently similar allergen cross-link two adjacent IgEs on sensitized mast cells or basophils. Activated mast cells or basophils subsequently degranulate releasing granules housing proinflammatory mediators and cytokines including histamine, tryptase LTC4, PGD2 thereby causing the clinical manifestations of allergy. Soluble allergens, sIgEs and mast cells or basophils are three key factors in the patho-physiological process of allergic inflammation, representing causative factors, messengers and primary effector cells, respectively. In contrast to primary effector cells, eosinophils and neutrophils are secondary effector cells, which can be accumulated and activated through the mediators released from mast cells or basophils. (See, e.g. He, S. H., Yang, P. C., et al., 2013, Acta pharmacologica Sinica, 34:1270-1283.) A similar sensitization response to that of respiratory allergies occurs when allergens bind to IgE bound mast cells in the gastrointestinal tract where there is an outflow of fluid across the gut epithelium induces vomiting and diarrhea. (See, e.g. Renz, H., Allen, K., Sicherer, S. et al., 2018, Nat Rev Dis Primers vol. 4, pp. 17098)

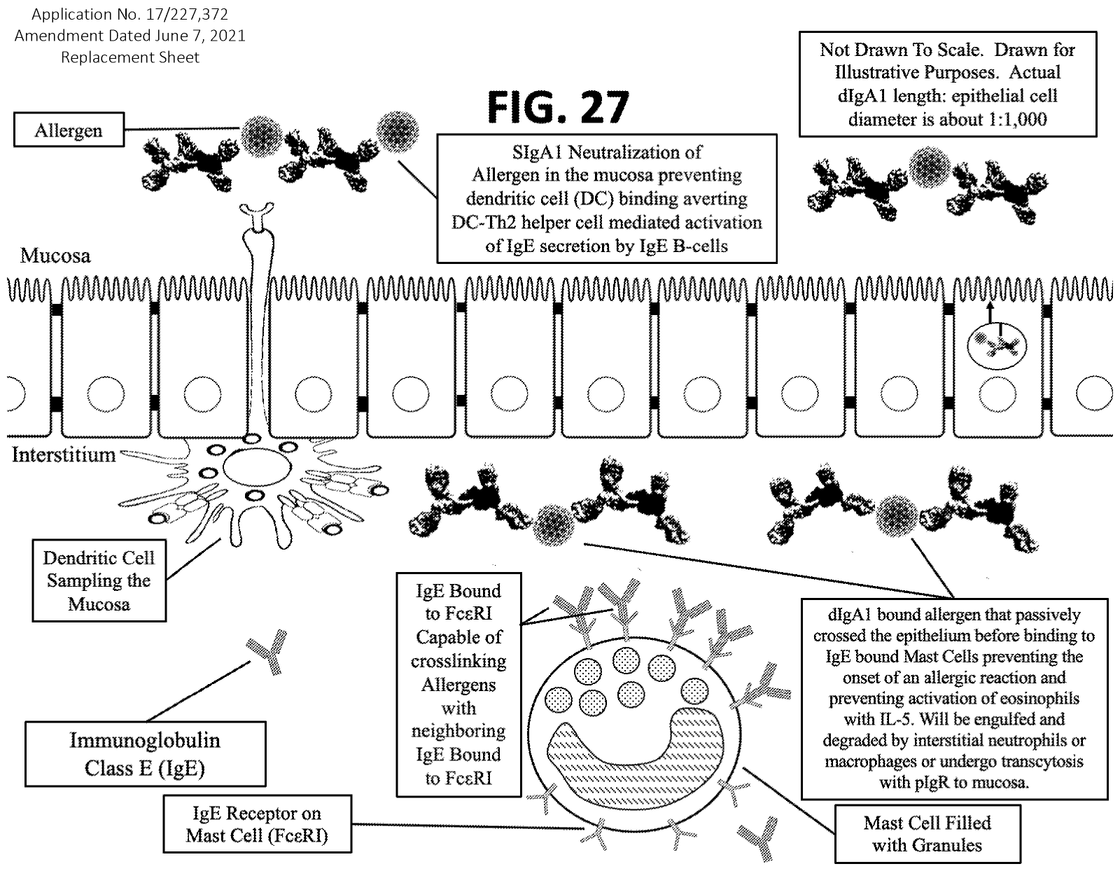

The immune response that leads to an excess of IgE production in response to an allergen is caused by two processes. One process signals cause naïve T cells to differentiate into T_{H}2 T-helper cells that produce cytokines that are specialized for promoting responses against parasites or extracellular bacteria. The second comprises T_{H}2 cells to stimulate B-cells to class switch to produce IgE. This results in higher levels of IgE in the interstitium of the lungs for example and the mast cells in the lung are subsequently heavily bound to IgE through the FcεRI receptor making the individual highly sensitive to that antigen the IgE is specific to making an individual asthmatic or results in GI disorders when the stomach is affected by the allergen. This instant patent contemplates blocking allergens from binding IgE by targeting the allergens. In one embodiment dIgA1 would provide mucosal protection in its SIgA1 form by binding to allergens in the respiratory mucosa and also in its dIgA1 form in the interstitium above and below the basement membrane providing three lines of protection against allergens binding IgE bound mast cells and one or two lines of protection—depending if the dendritic cell is sampling the mucosal environment—against allergens binding IgE bound dendritic cells (see FIG. 27). (See, e.g. Holgate, S., Wenzel, S., Postma, D. et al., 2015, Nat Rev Dis Primers vol. 1, pp. 15025)

In some embodiments dIgA1 encoded gene therapy specific to the allergen of interest is the therapeutic approach that prevents allergic reactions. Because dIgA (dIgA1 and dIgA2) is actively transported to the mucosa of the lungs and gastrointestinal tract with it becomes SIgA it can bind to allergens before they have a chance to bind to dendritic cell extensions in the mucosa or cross the mucosa and reach IgE bound mast cells that are largely responsible for the allergic response in asthma (See FIG. 27). Additionally, any allergens that cross the mucosal barrier will also be bound by dIgA1 in the interstitium. Thus, dIgA1 provides two levels of protection against allergens. The discovered potent dIgA1 immunoglobulins or immunoglobulin binding regions incorporated into a dIgA1 vector construct will code for dIgA1 and be able to bind to the allergen on multiple faces neutralizing the antigen and causing agglutination where macrophages and monocytes can degrade the antigen bound antibodies without causing allergies nor ADE or any adverse reactions.

### Cancer

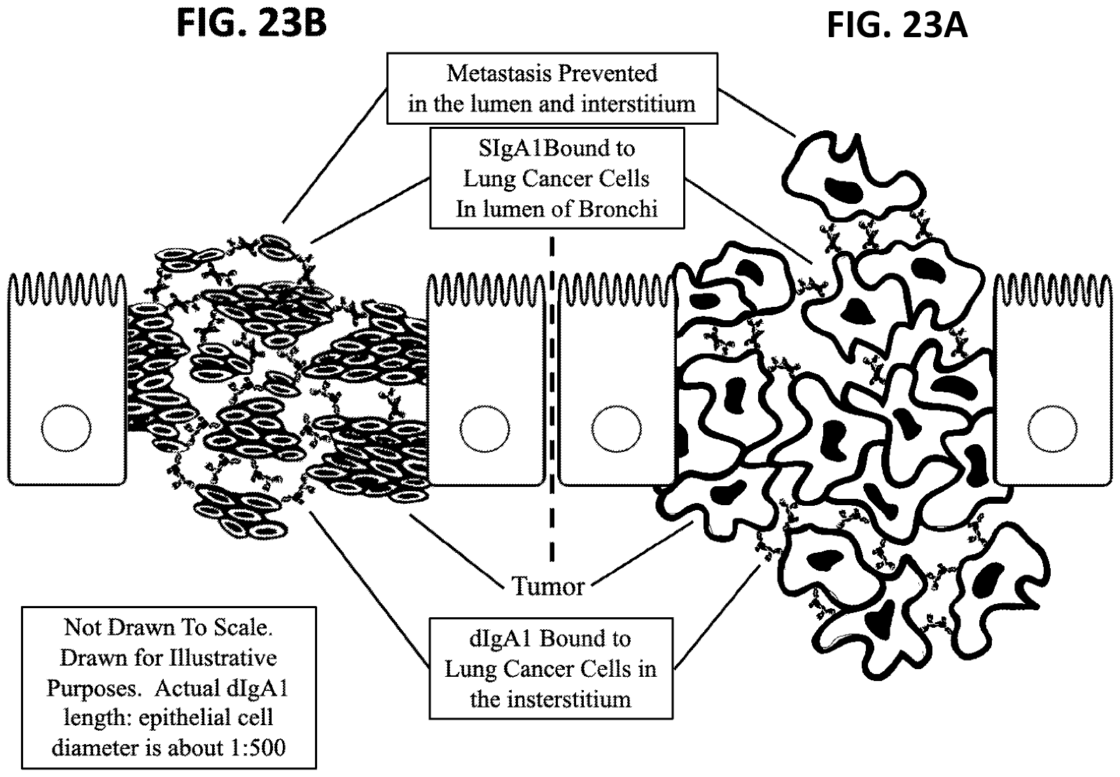

This patent contemplates the use of dIgA (dIgA1, dIgA2 and engineered variants) and even bispecific dIgA to target cancer. Cancer is a complex pathology that is made up of many different forms. Key characteristics of cancer are abnormal and nonspecific cell growth taking the place of healthy cells while not supporting organ function. The most common types of cancer in males include lung cancer, colorectal cancer, prostate cancer and stomach cancer. In females the most common types of cancer include breast cancer (80% invasive ductal carcinomas), lung cancer, colorectal cancer and cervical cancer. In other words most of the more common types of cancer involve mucosal or exocrine tissue and is often referred to as a carcinoma which makes up 80%-90% of all cancer cases. Thus, dIgA has privileged access to the malignancy in the mucosal lumen or exocrine duct to a greater degree than other immunoglobulins as a result of its active transport by polymeric immunoglobulin secreting receptor (pIgR) to the apical face of the epithelial cells lining the mucosal or exocrine tissue. A further benefit of dIgA and its SIgA counterpart over other immunoglobulins is that it can prevent “metathesis” or the spread of cancers cells to other areas of the body because of its two binding faces. When a cancer cell departs from the malignant tumor dIgA or SIgA in the mucosa or exocrine duct can capture the escaping tumor cell(s) by binding the cell on the immunoglobulin face that is opposite the immunoglobulin face bound to the malignancy (see FIGS. 23A and 23B). Further, pIgR is often upregulated and less often down regulated on epithelial cells proximal to the tumorous growth. One example of a defining feature of most cancers is an upregulation of secreted proteins and membrane bound proteins. The upregulation is significant enough that therapeutic antibodies are often used to mitigate the cancer despite healthy cells in the body also producing the same cell surface markers. In one embodiment the dIgA gene therapy will be administered to a lymph node proximal to the exocrine duct of the target organ. Thus, other immunoglobulins may only have access to part of the parts of the cancerous growth that are not facing into the lumen or exocrine duct or bordered by rather tall epithelial cells lining the mucosa and exocrine gland. Many organs with a mucosa and exocrine glands have their own lymphatic support system. A common therapeutic strategy to battle cancer includes use immunoglobulins to target cancer.

The epidermal growth factor receptor (EGFR) is a transmembrane protein that is implicated in cancer. Mutations that cause EGFR overexpression have been associated with a number of cancers including Adenocarcinoma of the lung is the most common type of lung cancer, colon cancer, epithelium tumors of the head and neck at about 80-100% of cases. Somatic mutations involving EGFR lead to its constant activation, which produces uncontrolled cell division. In a wide variety of tissues EGFR expression is at a low level. Excessive expression or activation of EGFR can induce malignancies or malignant transformations. EGFR is observed in 45-75% of non-small cell lung cancer (LSCLC) and is associated with aggressive clinical behavior including increased metastatic rate, high tumor proliferation and advanced stages of cancer. (See, e.g. Zhang, X., & Chang, A., 2007, Journal of medical genetics, vol. 44, pp. 166-172.) Non-small cell lung cancer remains the leading cause of cancer death in the U.S. and accounts for 85% of lung cancer cases in the U.S. The 5-year overall survival rate for patients with metastatic non-small cell lung cancer is 24%.

Another important receptor class involved in mucosal cancers is the PD-1/PD-L1 pathway which controls the maintenance and induction of immune tolerance within the tumor microenvironment. Programmed cell death protein 1 (PD-1) is a T-cell surface protein that regulates the immune response by down regulating the immune system and promoting self-tolerance. This pathway can thus attenuate the immune response of T cells to cancer cells. PD-1 has been targeted in immunotherapies to block the activity of this receptor in T-cells. Effectively, PD-1 antagonists can enhance the T-cell response against cancer cells and also blocks through competitive inhibition particular cancer cell receptors referred to as Programmed Death Ligand 1 (PD-L1) and PD-L2 from binding PD-1 on T-cells. When PD-L1 and PD-L2 bind T-cells they reduce T-cell survival and proliferation, in addition to reducing their cytokine secretion while making it more difficult for such PD-1 bound T-cells from being activated. It has been shown for example, that mice which lack PD-1 can steadily develop autoimmunity. Anti-PD-1/PD-L1 inhibitors have seen success in reducing cancerous tumor growths a number of mechanisms that including enhancing the T-cell response, activating a series immune system responses that combat tumor cells in addition to inducing infiltration into tumors. Additionally, CTLA-4 (CD152) agonist also controls the proliferation of T-cells and thus binding CTLA-4 with an antagonist enhances T-cell proliferation in the presence of cancer. It has also been shown that CTLA-4 activated T-cells can inhibit the proliferation of other T-cells.

TABLE 2
Table of 5′ Human UTRs

| SEQ | | | |
|---|---|---|---|
| ID NO: | 5′ UTR | Length | Protein |
| 42 | agaagaagtg aagtcaag | 18 | J Chain |
| 43 | tgagcgcaga aggcaggact | 36 | Immunoglobulin |
| | cgggacaatc ttcatc | | Lamba Variable |
| | | | Variant 1 |
| 44 | gctgcgggta gagaagacag | 40 | Immunoglobulin |
| | gactcaggac aatctccagc | | Lamba Variable |
| | | | Variant 2 |
| 45 | gcaggaatca gtcccactca | 29 | Immunoglobulin |
| | ggacacagc | | Kappa Variable |
| | | | Variant 1 |
| 46 | aggctggaca cacttcatgc | 53 | Immunoglobulin |
| | aggagtcagac cctgtcaggac | | Kappa Variable |
| | acagcatagac | | Variant 2 |
| 47 | gagagcatca cccagcaacc | 63 | Immunoglobulin |
| | acatctgtcc tctagagatc | | Heavy Variable |
| | ccctgagagc tccgttcctc | | Variant 1 |
| | acc | | |
| 48 | agtgactcct gtgccccacc | 20 | Immunoglobulin |
| | | | Heavy Variable |
| | | | Variant 2 |
| 49 | actcttctgg tccccacaga | 37 | hemoglobin |
| | ctcagagaga acccacc | | subunit alpha 1 |
| 50 | actcttctgg tccccacaga | 37 | hemoglobin |
| | ctcagagaga acccacc | | subunit alpha 2 |
| 51 | gacagtgctg acactacaag | 47 | fibrinogen- |
| | gctcggagct ccgggcactc | | gamma chain |
| | agacatc | | |
| 52 | aagtctac | 8 | fibrinogen- |
| | | | beta chain |
| 53 | aatcctttct ttcagctgga | 55 | fibrinogen- |
| | gtgctcctca ggagccagcc | | alpha chain |
| | ccacccttag aaaag | | |
| 54 | ctagcttttc tcttctgtca | 41 | albumin |
| | accccacacg cctttggcac a | | |

TABLE 3
Table of 3′ Human UTRs

| SEQ | | | |
|---|---|---|---|
| ID | | | |
| NO: | 3′ UTR | Length | Protein |
| 55 | tttaagtcat tgctgactgc atagctcttt ttcttgagag gctctccatt ttgattcaga | 788 | J Chain |
| | aagttagcat atttattacc aatgaatttg aaaccagggc tttttttttt ttttgggtga | | |
| | tgtaaaacca actccctgcc accaaaataa ttaaaatagt cacattgtta tctttattag | | |
| | gtaatcactt cttaattata tgttcatact ctaagtatcaa aatcttccaa ttatcatgct | | |
| | cacctgaaag aggtatgctc tcttaggaat acagtttcta gcattaaaca aataaacaag | | |
| | gggagaaaat aaaactcaag gactgaaaat caggaggtgt aataaaatgt tcctcgcatt | | |
| | cccccccgct tttttttttt tttttgactt tgccttggag agccagagct tccgcatttt | | |
| | ctttactatt ctttttaaaa aaagtttcac tgtgtagaga acatatatgc ataaacatag | | |
| | gtcaattata tgtctccatt agaaaaataa taattggaaa acatgttcta gaactagtta | | |
| | caaaaataat ttaaggtgaa atctctaata tttataaaag tagcaaaata aatgcataat | | |
| | taaaatatat ttggacataa cagacttgga agcagatgat acagacttct ttttttcata | | |
| | atcaggttag tgtaagaaat tgccatttga aacaatccat tttgtaactg aaccttatga | | |
| | aatatatgta tttcatggta cgtattctct agcacagtct gagcaattaa atagattcat | | |
| | aagcata | | |
| 56 | gtgccacggc cggcaagccc ccgctcccca ggctctcggg gtcgcgcgag gatgcttggc | 154 | IgHG1 Immunoglobulin |
| | acgtaccccg tgtacatact tcccaggcac ccagcatgga aataaagcac ccagcgcttc | | Heavy Constant Chain |
| | cctgggcccc tgcg | | |
| 57 | gtgccacggc cggcaagccc ccgctcccca ggctctcggg gtcgcgcgag gatgcttggc | 135 | IgHG2 Immunoglobulin |
| | acgtaccccg tctacatact tcccgggcac ccagcatgga aataaagcac ccagcgctgc | | Heavy Constant Chain |
| | cctgggcccc tgcga | | |
| 58 | gtgccacggc cggcaagccc ccgctcccca ggctctcggg gtcgcgcgag gatgcttggc | 135 | IgHG3 Immunoglobulin |
| | acgtaccccg tctacatact tcccgggcac ccagcatgga aataaagcac ccagcgctgc | | Heavy Constant Chain |
| | cctgggcccc tgcga | | |
| 59 | gtgccacggc cggcaagccc ccgctcccca ggctctcggg gtcgcgcgag gatgcttggc | 135 | IgHG4 Immunoglobulin |
| | acgtaccccg tctacatact tcccgggcac ccagcatgga aataaagcac ccagcgctgc | | Heavy Constant Chain |
| | cctgggcccc tgcga | | |
| 60 | gcaggagccg gcaaggcaca gggaggaagt gtggaggaac ctcttggaga agccagctat | 113 | IgHA1 Immunoglobulin |
| | gcttgccaga actcagccct ttcagacatc accgacccgc ccttactcac atg | | Heavy Constant Heavy A1 |
| | | | for memory B-cell |
| | | | Receptor |
| 61 | gccgcccgcc tgtccccacc cctgaataaa ctccatgctc ccccaagcag | 50 | IgHA1 Immunoglobulin |
| | | | Heavy Constant Heavy A1 |
| 62 | gcgggagccg gcaaggcaca gggaggaagt gtggaggaac ctcttggaga agccagctat | 144 | IgHA2 Immunoglobulin |
| | gcttgccaga actcagccct ttcagacatc accgacccgc ccttactcac gtggcttcca | | Heavy Constant Heavy A2 |
| | ggtgcaataa agtggcccca agga | | for memory B-cell |
| | | | Receptor |
| 63 | gccgcccgcc tgtccccacc cctgaataaa ctccatgctc ccccaagc | 49 | IgHA2 Immunoglobulin |
| | | | Heavy Constant Heavy A2 |
| 64 | gttcccaact ctaaccccac ccacgggagc ctggagctgc aggatcccag gggaggggtc | 140 | IgKL Immunoglovulin |
| | tctctcccca tcccaagtca tccagccctt ctccctgcac tcatgaaacc ccaataaata | | Kappa Light Constant |
| | tcctcattga caaccagaaa | | Chain |
| 65 | gttcccaact ctaaccccac ccacgggagc ctggagctgc aggatcccag gggaggggtc | 140 | IgλL Immunoglovulin |
| | tctctcccca tcccaagtca tccagccctt ctccctgcac tcatgaaacc ccaataaata | | Lambda Light Constant |
| | tcctcattga caaccagaaa | | Chain |
| 66 | gctggagcct cggtggccat gcttcttgcc ccttgggcct ccccccagcc cctcctcccc | 111 | HBA1 hemoglobin subunit |
| | ttcctgcacc cgtacccccg tggtctttga ataaagtctg agtgggcggc a | | alpha 1 |
| 67 | gctggagcct cggtagccgt tcctcctgcc cgctgggcct cccaacgggc cctcctcccc | 110 | HBA2 hemoglobin subunit |
| | tccttgcacc ggcccttcct ggtctttgaa taaagtctga gtgggcagca | | alpha 2 |
| 68 | aaaattatgt ctttttaata tggtttttgt tttgttatat attcacaggc tggagacgtt | 232 | fibrinogen-gamma chain |
| | taaaagaccg tttcaaaaga gatttacttt tttaaaggac tttatctgaa cagagagata | | |
| | taatattttt cctattggac aatggacttg caaagcttca cttcatttta agagcaaaag | | |
| | accccatgtt gaaaactcca taacagtttt atgctgatga taatttatct ac | | |
| 69 | actaagttaa atatttctgc acagtgttcc catggcccct tgcatttcct tcttaactct | 219 | fibrinogen-alpha chain |
| | ctgttacacg tcattgaaac tacacttttt tggtctgttt ttgtgctaga ctgtaagttc | | |
| | cttgggggca gggcctttgt ctgtctcatc tctgtattcc caaatgccta acagtacaga | | |
| | gccatgactc aataaataca tgttaaatgg atgaatgaa | | |
| 70 | catcacatt taaaagcatc tcagcctacc atgagaataa gagaaagaaa atgaagatca | 414 | Albumin |
| | aaagcttatt catctgtttt tctttttcgt tggtgtaaag ccaacaccct gtctaaaaaa | | |
| | cataaatttc tttaatcatt ttgcctcttt tctctgtgct tcaattaata aaaaatggaa | | |
| | agaatctaat agagtggtac agcactgtta tttttcaaag atgtgttgct atcctgaaaa | | |
| | ttctgtaggt tctgtggaag ttccagtgtt ctctcttatt ccacttcggt agaggatttc | | |
| | tagtttcttg tgggctaatt aaataaatca ttaatactct tctaagttat ggattataaa | | |
| | cattcaaaat aatattttga cattatgata attctgaata aaagaacaaa aacca | | |

### Lipid Nanoparticles

This instant patent contemplates Lipid Nanoparticles as delivery vehicles for mRNA encoding for dIgA1 or dIgA2. Lipid Nanoparticles have seen unprecedented advances in medicine. LNPs have advantages over capsid delivered vectors in that they are not immunogenic, are biodegradable, have an impressive safety profile and can be delivered with adjuvants. In addition, LNPs have control over drug release, facilitate endosomal escape and can be effective scaled up for manufacturing.

LNPs surface features such as the addition of antibodies are typically added after the formation of the LNP with its payload. LNPs range in size from 1 nm to 1000 nm. Although ideally LNPs are less then 150 nm to facilitate their ability to reach their target destination. Lipid nanoparticles can efficiently target liver cells (See e.g. Truong, B., et. al., 2019, PNAS, 116:21150-21159; Kim, M., et. al., 2021, 7:1-12, 2021.) LNPs can effectively be made to target any cell type through adding monoclonal antibodies or other binding proteins on their surface that are specific to the target cell surface receptor or other CD receptor. This can be done in a variety of ways such as modular manner where a (See, e.g. Veiga, N., Goldsmith, M., Granot, Y. et al., 2018, Nat Commun, 9:4493) LNPs are very effective at delivering the payload to the cytosol of the cell but not the nucleus of the cell. In one embodiment when mRNA is intended for translation in the rough ER or other destination that information can effectively be encoded for on the 3′ UTR. In another embodiment using the evolutionary conserved 3′ UTR for the immunoglobulin as an example or other 3′ UTR for example the 3′ UTR of a protein that is heavily expressed by humans or highly expressed and secreted in the target-cell can deliver the mRNA for increased protein yield.

LNPs are generally made up of an aqueous core surrounded by a lipid bilayer shell that can include a number of lipids that serve different functions. LNPs generally rely on cationic lipids to efficiently complex negatively charged RNA. Although, anionic and neutral formulations have proven to be effective. Cationic lipids bearing a permanent positive charge are more toxic. Although, amine groups can be incorporated into LNPs allowing them to maintain a cation surface charge at physiological pH which both reduces nonspecific lipid-protein interactions and also allows for RNA release in the cytosol. The cationic charge on the LNP surface allows for efficient interaction with the negatively charged membranes on cell surfaces. This in turn also facilitates engulfment by the cell where the resulting negatively charged endosome is disrupted by the positively charged LNP that facilitates escape of the mRNA. Phospholipids also contribute to the disruption of the endosome. (See e.g., Reichmuth, A. M., Oberli, M. A., Jaklenec, A., Langer, R., Blankschtein, D., 2016, Therapeutic delivery, 7:319-334) There are an extensive number of methods involved in incorporating mRNA in LNPs. In one of many approaches mRNA molecules self-assemble in ionizable molecules in acidic conditions to form uniform LNPs by combining the ingredients of LNPs with the drug. In another approach preassembled LNPs are combined with the drug through a directed microfluidic integration process. The method used in LNP synthesis influences the LNP size and encapsulation efficiency. One method to form LNPs involves condensing lipids from an ethanol solution in water. Aqueous phase mRNA may be encapsulated during condensation. Lipid rafts may be converted into LNPs in a controlled microfluidic process that can accelerate or decelerated the rate of LNP closure influencing the size of the LNP and in turn the total mRNA packaged within it. (See e.g., Reichmuth, A. M., Oberli, M. A., Jaklenec, A., Langer, R., & Blankschtein, D., 2016, Therapeutic delivery, 7:319-334)

### Chimeric Antibodies

This instant patent contemplates the use of chimeric antibodies in the dIgA1 and dIgA2 gene therapy constructs as an effective means to develop potent affinity for the target of interest. Using the immune system of Mice can be an effective means to develop potent binding affinity against the target of interest. However, attempting to take an antibody discovered in mouse for use in human often results in a human anti-murine antibody response. Although, there is are a few examples of a fully murine FDA approved monoclonal antibody for cancer and kidney transplant rejection. This response can be to the constant region of the mouse antibody or even part of the V-region of the mouse antibody. As a result potent antibodies developed in mice have had all or part of the genetic information coding for the binding region incorporated in a human antibody construct that preferably utilizes all of the constant region from one human antibody heavy chain (IgG1, IgG2, IgG3 and IgG4) and part of the V-region that the binding element of the mouse antibody can tolerate without losing binding affinity to its target. This patent contemplates the same hybridization techniques to be used in IgA1 and IgA2 constructs intendent for chimeric dIgA1 and dIgA2 gene therapy encoded vectors. Further, such hybridization techniques may use any mouse IgG or IgA antibody V region and incorporate the necessary components of the V region into an IgA1 or IgA2 antibody. All antibody heavy chains are based on V-regions derived from the same genetic loci and subjected to the same conditions related to affinity maturation making dIgA1 and dIgA2 highly suitable for any chimeric antibody derived from a mouse or other non-human vertebrate. Additionally, there is no significant different in the principle constructs and function behind IgA1 or IgA2 and that of IgG1, IgG2, IgG3 and IgG4 in their engagement with the V-region. That is a V-region that is effective in one construct is generally effective in all constructs.

In antibodies the residues in the variable domains (V region) in each of the heavy and light chains of the region are partitioned into three hypervariable complementarity-determining regions (CDRs) and four framework regions (FRs). The FRs, which make up about 85% of the V region, are a more stable amino acid sequence and function as the scaffolds for the CDRs that directly contact the antigen. The FRs can influence the orientation, relative conformation, phi an psi angles of the amino acids in the CDRs and thus can ultimately improve or disrupt the binding affinity of the CDR derived from a mouse monoclonal antibody. Thus, some of the mouse FRs may be used in the chimeric antibody with potentially some amino acid substitution in the human FRs or non-human vertebrate derived CDRs may be made to ensure the binding affinity remains potent. In one embodiment the CDRs from a mouse IgG1, IgG2A, IgG2B, IgG2C or IgA antibody are incorporated into an otherwise fully human IgA1 or IgA2 antibody intended to be expressed as dIgA1 or dIgA2 respectively. In another embodiment all the CDRs and one or more of FR1, FR2, FR3 and FR4 from a mouse IgG1, IgG2A, IgG2B, IgG2C or IgA antibody are incorporated into an otherwise fully human IgA1 or IgA2 antibody intended to be expressed as dIgA1 or dIgA2 respectively. In a further embodiment amino acid substitutions, additions or deletions are made into the CDRs or FRs from mice or human in the chimeric dIgA1 or dIgA2 antibodies. In a further embodiment another non-human vertebrate such as a rat or rabbit is used in place of a mouse.

Mice or Other Non-Human Vertebrate with Engineered Immune Systems (Transgenic Mice):

This instant patent contemplates the use of mice with engineered immune systems or other non-human vertebrate with engineered immune systems so that it produces fully human antibodies in place of the murine or non-human vertebrate antibodies is an important for the discovery of antibodies with therapeutic potential. Engineered animals such as mice for this purpose are capable of producing a fully antibody repertoire capable of affinity maturation towards the antigen target. Engineered animals are capable of highly evolved in-vivo mechanisms such as hypermutation in germinal centers resulting in fully human (comprising human variable and constant regions) high affinity antibodies with optimal biophysical properties that because they are developed in vivo in the host non-human vertebrate are less likely to cause immunogenicity in humans. Use of non-human vertebrates with engineered immune systems provides a straight-forward approach to rapidly obtain antigen specific antibodies that have undergone recombination, junctional diversification, affinity maturation and isotype switching in vivo in a non-human vertebrate system. Fully human antibodies avoid the problem of attenuating antibody characteristics when humanizing the constant region of chimeric antibodies and thus can lead to more exquisite specificity for the target with reduced overall effort and cost. The earliest in vivo non-human vertebrate used for this purpose in vivo was Xenomouse™ that used completely human transgenic heavy chain loci which comprise human variable regions (human V_{H}, D and JH gene segments) upstream of human constant regions. Subsequently, it has been discovered that the use of 60 totally human transgenic loci in such in vivo systems is detrimental and B-cell development is hampered, leading to relatively small B-cell compartments and restricted utility for generating antibodies. Later-generation transgenic animals (eg, the Velocimouse™) have been created which have 65 chimeric heavy chain loci in which a human variable region is upstream of endogenous (eg, mouse or rat) constant expression. US2008/0196112A1 (Innate Pharma) discloses transgenic animals comprising a single, predetermined human rearranged VDJ from a lead antibody, together with one or more human constant region genes in a locus. (See e.g. For example, see U.S. Pat. Nos. 10,251,377 B2 and 10,667,501 B2 expressly incorporated by reference herein in their entirety; Also see Lee, E. C., Liang, Q., Ali, H. et al., 2014, Nat. Biotechnol., 32:356-363). These mice with engineered immune systems serve as an efficient means to discover immunoglobulins and immunoglobulin V regions that are potent for the target of interest. Potent antibodies may be discovered through FACS cell sorting or magnetic beads biotinylated to the antigen of interest. In one embodiment the incorporation of such V regions is made into dIgA1 and dIgA2 DNA and mRNA vector constructs.

### Tissue Targeting

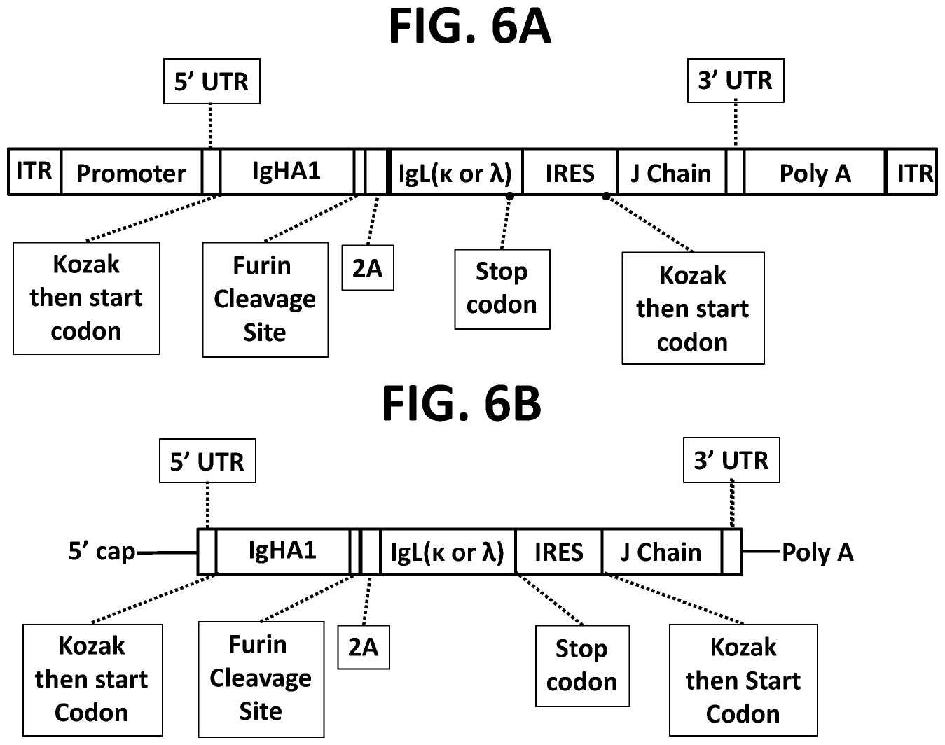

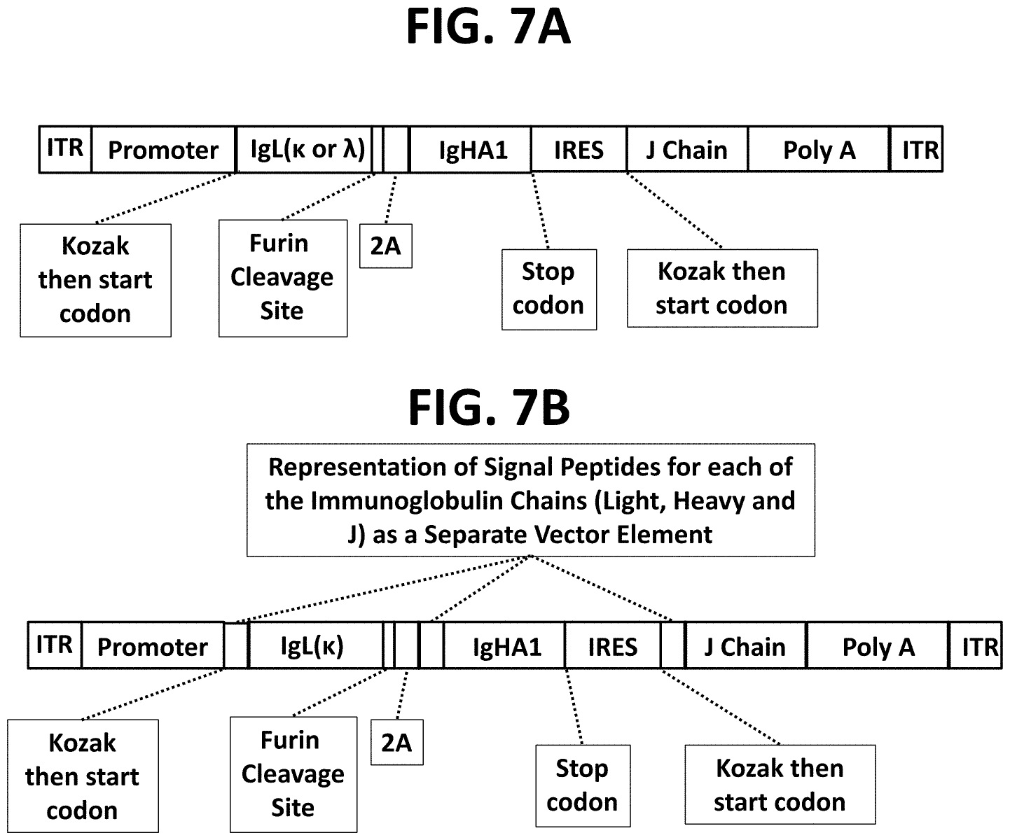

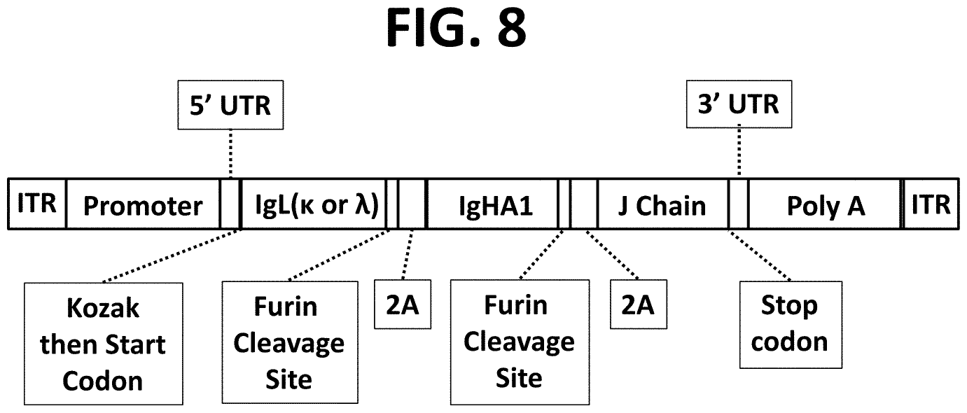

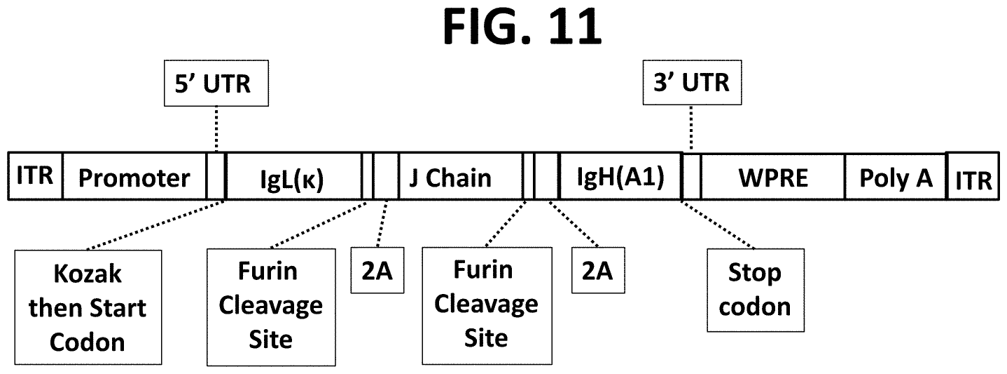

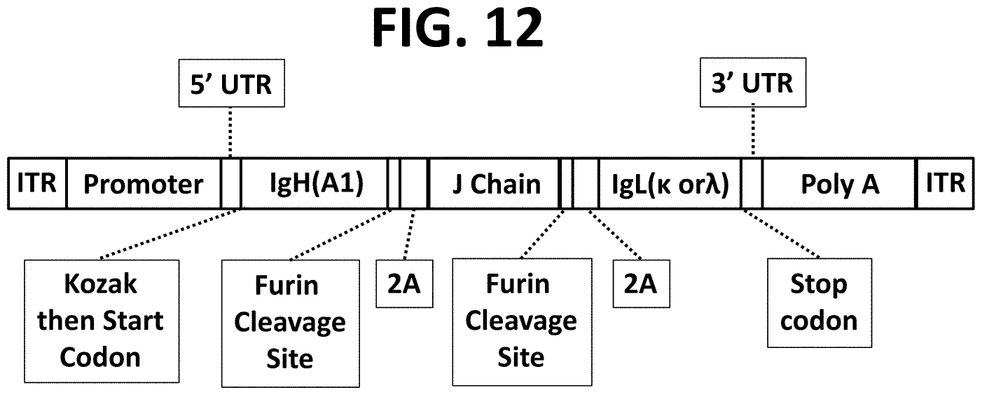

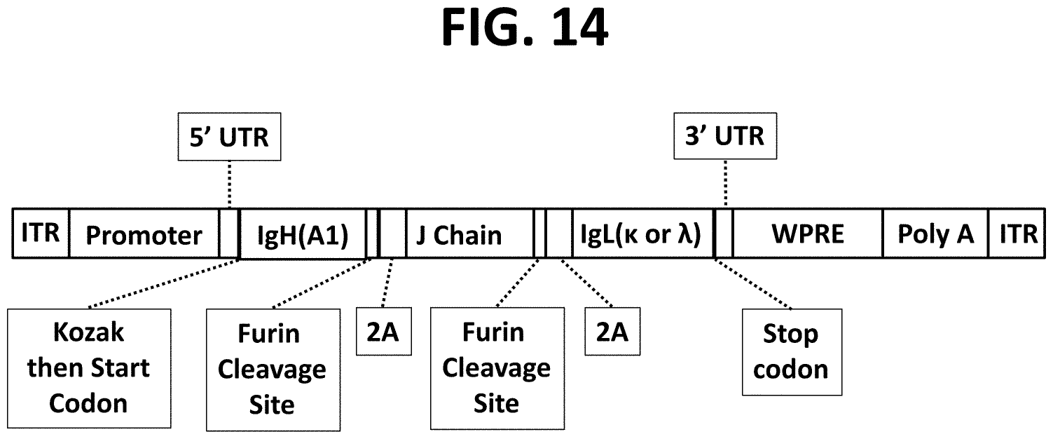

The instant patent invention contemplates the delivery of an episomally-maintained gene therapy based vaccination/immunization as an intramuscular injection, intravenous injection or injection proximal to lymph nodes that can be an intramuscular injection and administration to the lamina propria via endoscopic injection. AAV vectors (See FIGS. 6, 7, 8, 9, 11, 12, 13, 14, 15 and 16 as examples) may be delivered intramuscularly where that AAV capsid may transduce the skeletal muscle cell (myocyte) and deliver the vector to muscle nuclei where the vectors may form concatemers or reside as monomeric circular gene elements. Skeletal muscles are particularly beneficial because they are a non-dividing cell population. Thus, episomes not capable of self-replication may persist for years in the skeletal muscle nuclei. Also, targeted in the liver are hepatocytes that have half-lives of 200-400 days and thus are ideal for longer term expression of dIgA1 and dIgA2. AAV capsids are also effective at targeting hepatocytes. In one embodiment naked vector DNA is delivered to muscle cells with the use of electroporation. It has been shown that electroporation increases gene transfection efficiency to the muscle nuclei by 100 fold. Electroporation may be defined in this instance as low voltage pulses to the muscle, which allows macromolecules such as DNA vectors to enter the cells more efficiently. (See e.g., Tjelle, T. E., et. al., 2004, Mol. Ther., 9:328-336; Andrews, C. D., et al., 2017, Methods and Clinical Development, 7:74-82) Additionally, pseudotyped lentiviral vectors and or lipid nanoparticles with site specific targeting ligands may target liver cells (hepatocytes) bearing specific CD receptors. In another embodiment hepatocytes are targeted such as with AAV8 another privileged cell type capable of expressing Immunoglobulins (See e.g. Lei, Y., Huang, T., Su, M. et al., 2014, Lab Invest 94:1283-1295) and have a half-lives of 200-400 days.) In a further embodiment the lamina propria of the lungs e.g. trachea or the stomach and duodenum are be targeted to deliver lentiviral vectors with the aim of delivering such vectors to the germinal center memory B-cells in the supporting lymph nodes and memory B-cells in the interstitium for the particular tissue. For further invention disclosures on targeting the lamina propria see the section on “The Interstitium of the Lamina Propria”.

### Viral Vectors, Non-Viral Vectors and Retroviral Vectors

The vectors of the invention include heterologous control sequences, which include, but are not limited to, constitutive promoters, such as the human cytomegalovirus (CMV) immediate early enhancer/promoter (SEQ ID NO. 1), Rous sarcoma virus (RSV), simian virus 40 (SV40) and mammalian elongation factor 1α (EF1α), are non-specific promoters and are commonly used in gene therapy vectors. Other promoters that are commonly used in gene therapy include cytomegalovirus enhancer/chicken beta-actin (CAG). Other promoters include mouse phosphoglycerate kinase (mPGK) and human synapsin (hSYN) promoter. Preferred promoters include the EF1-alpha promoter (Kim et al., Gene 91(2): 217-23 (1990)) and Guo et al., Gene Ther. 3(9):802-10 (1996)). Highly preferred promoters include the human cytomegalovirus (CMV) immediate early gene enhancer/promoter, elongation factor 1-alpha (EF1a) promoter, a cytomegalovirus enhancer/chicken beta-actin (CAG) promoter and a simian virus 40 (SV40) promoter. Other promoters include a tetracycline responsive promoter (TRE), a transthyretin promoter (TTR), and a CK6 promoter. The sequences of these and numerous additional promoters are known in the art. The relevant sequences may be readily obtained from public databases and incorporated into vectors for use in practicing the present invention. In some cases the relevant sequences are published in a Journal Article.

Typically, in order to express 2 or more proteins in a vector two or more promoters are required. As an example, an internal ribosome entry site (IRES) sequence is often used to drive expression of individual genes when those genes are located 5′ to at least one gene. Alternatively, an elongation factor 1-alpha promoter (EF1α) may be used where a polyadenylation site may be used to process the mRNA of the preceding gene. When two genes are linked with an IRES sequence, the expression level of the second gene is often significantly weaker than the first gene (Furler et al., Gene Therapy 8:864-873, 2001). However, one may use as will be understood by those of skill in the art, expression vectors typically include a promoter operably linked to the coding sequence or sequences to be expressed, as well as ribosome binding sites, RNA splice sites, a polyadenylation site, and transcriptional terminator sequences, as appropriate to the coding sequence(s) being expressed. Other sequences such as a Kozak sequence (SEQ ID NO: 20) such as “GCCACC” are introduced directly before the transcription start codon to allow for the expression of a transgene. The Kozak sequence is also important for the second transcription start codon that is directly preceded by an IRES or directly preceded by a second promoter. In general the Kozak sequence is important for the translation initiation at any place on the mRNA.

Increased levels of gene expression generally occurs when a Woodchuck hepatitis-virus posttranscriptional regulatory element (WPRE) is incorporated. WPRE is most effective when placed downstream of the transgene, following the 3′ UTR proximal to the polyadenylation signal. Polyadenylation signals and WPRE are incorporated into vectors to ensure high levels of gene expression. In some embodiments WPRE is incorporated into the vectors. In other embodiments two consecutive WPRE regulatory elements are used. Generally, WPRE and a polyadenylation signal can substantially increase gene expression over the vector that only includes the polyadenylation signal.

### Prolonged Gene Expression

Episomally maintained vectors with self-replicating elements are capable of long-term episomal persistence in human cells and may be referred to as episomally-maintained self-replicating vectors. Typically, these vectors are based on few designs. One common design contains components derived from either EBV or bovine papilloma virus, where there is a greater emphasis for gene therapy related applications with EBV. EBV is a human herpesvirus that can maintain its genome extra-chromosomally as an episome in dividing mammalian cells. Maintenance is also achieved with the EBV viral latent origin of replication oriP and EBV nuclear antigen 1 (EBNA1) that act together to replicate and retain the viral genome in the nucleus. Thus, inclusion of these sequences allows them to be maintained in dividing cells. (See e.g., Conese, M., et. al., 2004, Gene Ther., 11:1735-1741) Although, there are substantial safety and immunotolerance challenges associated with the EBNA1 protein making it unsuitable for present application.

### Vectors for Use in Practicing the Invention

The present invention contemplates the use of non-viral vectors, viral vectors and retroviral vectors as an efficient and effective means to express immunoglobulins whose polypeptide sequence is derived from humans especially human memory B-cells or plasmablasts, a transgenic animal, a mouse with humanized immune system, or a mouse any of which that was infected with, exposed to, has immune specificity to, or lives with any of (1) a virus (s) (2) a systemic ailment such as allergies (3) fungi (4) bacterial infection (4) cancerous tumor (5) biowarfare agent (6) microbial infection, (7) any ailment 8) an allergen or cause of allergies, (9) a target protein or variant (10) an immune system protein such as Immunoglobulin class E (IgE) or a cytokine. Additionally, scFv or Fab identified from phage display technology may be used to identify such potent immunoglobulin V or Fab regions. In another context only the V-regions (V_{L} and V_{H}), CDR regions or Fab regions may be used from humans B-cells, B-cells from transgenic mice, B-cells from mice or other non-human vertebrate with humanized immune systems and in some cases portions of the constant regions such as C_{H}1 and C_{L} polypeptide sequence are derived from the immunoglobulin polypeptide sequence of memory B-cells or plasmablasts. This strategy provides flexibility to use entire immunoglobulin polypeptide sequences or only V-region sequences in conjunction with other constant region sequences or including isotype switched constant region sequences, engineered constant region sequences that cannot be detected by Fc receptors and even a combination of constant region sequences from two sources. E.g. using the C_{H}1 and optionally the hinge polypeptide sequence and also the C_{L} sequences derived from potent immunoglobulins where the immunoglobulin heavy and light chain genes or polypeptide sequences were identified while using C_{H}2 and C_{H}3 polypeptide sequences from another human source. Additionally, CDRs may be used in conjunction with FR regions from another human or even mouse source. When mice are used to identify potent immunoglobulins for chimeric immunoglobulins will be systematically designed.

This patent contemplates an approach undertaken so that at risk individuals and individuals that may not be able to develop sufficient adaptive immunity quickly enough if at all to any of (1) a virus (s) (2) a systemic ailment such as allergies (3) fungi (4) bacterial infection (4) cancerous tumor (5) biowarfare agent (6) microbial infection, (7) any ailment (8) an allergen or cause of allergies (9) a target protein or variant (10) an immune system protein such as Immunoglobulin class E (IgE) or a cytokine, can be engendered immunity or immune protection in a relatively short time window in a manner that circumvents their adaptive immune system.

In the invention the vector constructs non-viral, viral and retroviral delivery systems then effectively serve as the mechanism to realize that immunoglobulin-based binding affinity that was discovered in humans, transgenic mice or other non-human vertebrate with an engineered immune system, mice with a resulting chimeric antibody, or mice with humanized immune systems when considering the expression of dIgA1 and dIgA2. However, the present invention further considers the use of vector constructs to express dimeric immunoglobulin A1 (dIgA1) and dIgA2 that encode for the immunoglobulin heavy chain, the immunoglobulin light chain (kappa or lambda), J chain and optional use of MZB1 in some embodiments on a single vector construct in order to also engender recipients with a mucosal immunity against the pathogen of interest. In these contexts the present invention considers the use of a variety of vectors to introduce DNA constructs that comprise these coding sequences. The expression of dIgA1 or dIgA2 in a single vector construct is important since a mix of different episomes that encode for a polyclonal mix of immunoglobulins potent against the pathogen of interest and likely to prevent mutated form of the pathogen from infecting the recipient of the episomal gene therapy can be rendered less effective if two such gene elements were used to express dIgA1. If dIgA required two vector constructs for encoding its expression such a strategy would require a disproportionately large titer of the vectors containing J chain and MZB1 to ensure that a sufficient portion of the same cells receive both vectors.

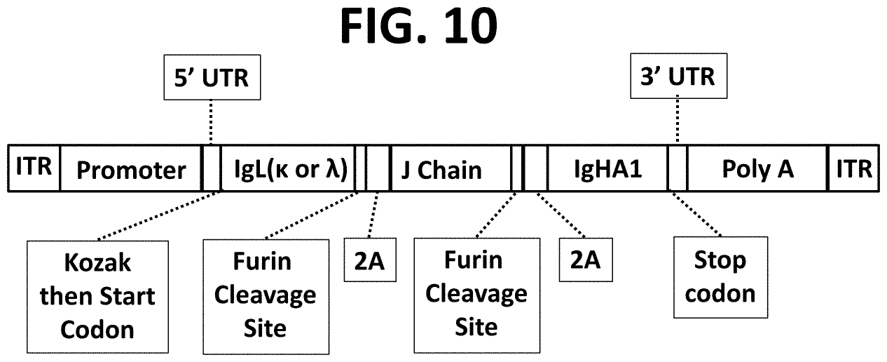

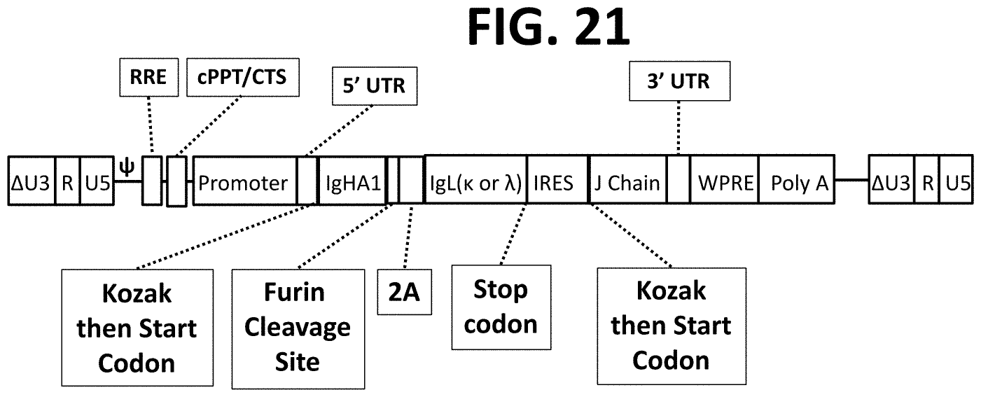

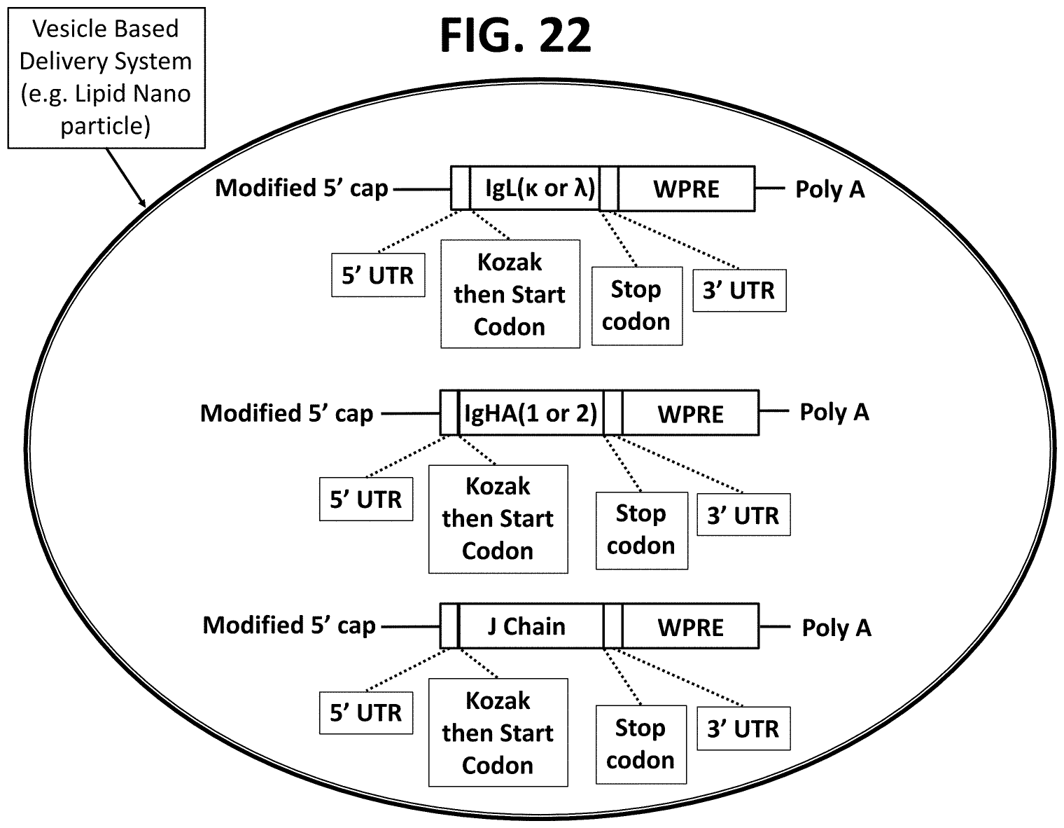

Any of a variety of vectors for introduction of constructs (See FIGS. 6 through 24 as examples) comprising the coding sequence for immunoglobulins that in some embodiments includes the use of 2A self-cleaving peptide sequences and furin cleavage sites. There are an extensive number of examples of gene expression vectors that are known in the art and such vectors may be viral and non-viral as well as self-replicating or replication deficient. Although, the use of self-replicating vectors to express immunoglobulins with integration competent lentiviral based gene delivery methods excludes the use of rAAV vectors that have a 4.9 Kb capacity. Non-viral, viral and retroviral gene delivery methods that may be employed in the practice of the invention include but are not limited to plasmids, lipid nanoparticles, liposomes, nucleic acid/liposome complexes and cationic lipids.

Reference to a vector as “recombinant” refers to the linkage of DNA sequences which are not known in nature and which come from two or more organisms in nature. Reference to the “transgene” refers to the gene encoding for a polypeptide in the vector that is intended for some function in the organism that is unrelated to the maintenance or support of the vector function. Expression of the transgene is regulated by sequences that are operatively linked to a polypeptide coding sequence when the expression and/or control sequences regulate the transcription and/or translation of the nucleic acid sequence. Thus expression and/or control sequences can include promoters, enhancers, transcription terminators, polyadenylation sites, furin cleavage sites, 2A self-cleaving polypeptides, replicating elements, a start codon (e.g., ATG) 5′ to the coding sequence, Kozak sequences, splicing signals for introns, a 5′UTR, a 3′UTR and stop codons. (For example, see U.S. Pat. Nos. 7,498,024 B2 and 7,709,224 B2 expressly incorporated by reference herein in their entirety)

Recombinant Adeno Associated Virus (rAAV) vectors are established to exhibit strong transient expression with excellent titer and the ability to enter dividing and non-dividing human cells without eliciting an immune response for most—upon first time exposure—to the AAV capsid protein structure. However, multiple exposures to an AAV capsid may elicit an immune response. Although, advances in science may likely overcome the immunogenicity of AAV that are also able to attain the same safety profile as currently clinically approved rAAVs. rAAV vectors delivered via AAV capsids have the ability to enter cells that depends in part on AAV serotype. Further, AAVs are favored for in vivo episomal gene transfer of therapeutic genes because of their low immunogenicity, strong safety record, and high efficiency of transduction of a number of cell types in animal models. (Wu, Z., et. al., 2006, Mol Ther. 14:316-327.) Replication-defective AAV virions encapsulating the recombinant AAV vectors of the instant invention are made by standard techniques known in the art using AAV packaging cells and packaging technology. The coding sequence for two or more polypeptides or proteins of interest is commonly inserted into the adenovirus in the deleted E3 region of the virus genome. The recombinant vectors of this invention comprise the ability to express dimeric Immunoglobulin A (dIgA), which is a tetramer of heterodimers linked together by disulfide bonds between immunoglobulin light and heavy chains and disulfide bonds between the J chain protein and the immunoglobulin heavy chain. Further the recombinant vectors of this invention encode for immunoglobulins that are naturally developed in humans especially human memory B-cells or plasmablasts, discovered from a transgenic animal, a mouse with humanized immune system, or a mouse any of which that was infected with, exposed to, has immune specificity to, or lives with any of (1) a virus (s) (2) a systemic ailment such as allergies (3) fungi (4) bacterial infection (4) cancerous tumor (5) biowarfare agent (6) microbial infection, (7) any ailment 8) an allergen or cause of allergies, (9) a target protein or variant (10) an immune system protein such as Immunoglobulin class E (IgE) or a cytokine. In some embodiments only a portion—that must include one or more of the CDRs and V-region segments of both the immunoglobulin light and heavy chains—of the polypeptide sequence encoding for the potent immunoglobulins will be used in the vector construct. The elements of the recombinant AAV vectors may comprise (A) a packaging site that enables the vector to be placed in replication incompetent AAV virions; (B) the coding sequence for immunoglobulin gene elements e.g. heavy chain (IgH), light chain (IgL), J chain and associated proteins e.g. MZB1; (C) the optional use of the sequence encoding for a 2A self-cleaving peptide site located C-terminal to a furin cleavage site. (D) a modified 2A self-cleaving peptide may be substituted for a full length 2A self-cleaving peptide to minimally contain the consensus sequence (C) in some cases with potentially some loss in efficiency (E) the optional use of a multi-promoter rAAV vector for immunoglobulin expression. (F) the optional use of both (C) and (D) in a single vector. (See FIGS. 6A, 7A, 7B, 8, 9A, 10, 11, 12, 13, 14, 15 and 16 as examples) Other elements necessary for or which improve the formation and/or function of the vectors or vector packaging including ITRs. All these methods are well within the practice of those skilled in the art. (For example, see U.S. Pat. Nos. 7,498,024 B2 and 7,709,224 B2)

Adeno-associated virus (AAV) is a helper-dependent human parvovirus, which is able to infect cells latently by chromosomal integration or delivery of an episomally maintain gene element to the nucleus. AAV has significant potential as a human gene therapy vector. For use in practicing the present invention rAAV virions may be produced using standard methodology, known to those of skill in the art and are constructed such that they include, as operatively linked components in the direction of transcription, control sequences including transcriptional initiation and termination sequences, and the coding sequence(s) of interest. More specifically, in the invention in one embodiment the recombinant AAV vectors of the instant invention comprise: (1) a packaging site enabling the vector to be incorporated into replication-defective AAV virions; (2) the coding sequence for two or more polypeptides or proteins of interest, e.g., heavy and light chains of an immunoglobulin of interest; (3) the optional use of a sequence encoding a 2A self-processing cleavage site alone or in combination with an additional furin cleavage site. (4) optional use of separate promoters and regulatory elements for each coding sequence (5) Optional use of a combination of (3) and (4). AAV vectors for use in practicing the invention are constructed such that they also include, as operatively linked components in the direction of transcription, control sequences including transcriptional initiation and termination sequences. In another embodiment these components are flanked on the 5′ and 3′ end by functional AAV ITR sequences. By “functional AAV ITR sequences” is meant that the ITR sequences function as intended for the rescue, replication and packaging of the AAV vectors. Further, AAV vectors for use in practicing the invention in one embodiment will code for (A) immunoglobulins that are derived from immunoglobulins identified from the blood of leukocytes from humans especially human B-cells, memory B-cells or plasmablasts, discovered from a transgenic animal, a mouse with humanized immune system, or a mouse any of which that was infected with, exposed to, has immune specificity to, or lives with any of (1) a virus (s) (2) a systemic ailment such as allergies (3) fungi (4) bacterial infection (4) cancerous tumor (5) biowarfare agent (6) microbial infection, (7) any ailment 8) an allergen or cause of allergies, (9) a target protein or variant (10) an immune system protein such as Immunoglobulin class E (IgE) or a cytokine. Or such FVsc identified from Phage Display technology. (B) immunoglobulins whose CDR or V-regions (V_{L} and V_{H}) are derived from immunoglobulins identified in identified from any of the same sources discussed in part (A) of this section. (C) immunoglobulins whose antibody binding fragments (Fab): V-regions (V_{L} and V_{H}) and constant heavy domain 1 C_{H}1 and constant light (C_{L}) chain domain are derived from immunoglobulins identified from any of the same sources discussed in part (A) of this section. (D) immunoglobulins whose antibody binding fragments that results from pepsin cleavage (Fab′)₂: V-regions (V_{L} and V_{H}) and constant heavy region 1 C_{H}1, hinge and constant light (C_{L}) chain domain are derived from immunoglobulins identified from any of the same sources discussed in part (A) of this section. (E) Dimeric Immunoglobulin A (dIgA) that requires cDNA coding for or the polypeptide sequence of the immunoglobulin class A immunoglobulin heavy and light chains, J chain with optional use of the signaling peptide (see SEQ IDs 11 and 7) and optional encoding of MZB1. (For similar examples, see U.S. Pat. Nos. U.S. Pat. No. 7,498,024 B2 and U.S. Pat. No. 7,709,224 B2; Also see e.g., Fang, J., et al., 2005, Nat. Biotechnol., 23: 584-590.; Krysan, D. J., et. al., 1999, J Biol Chem. 274:23229-23234)

Recombinant AAV (rAAV) vectors are capable of directing the expression and production of selected recombinant polypeptide or protein products in target cells. Thus, the recombinant vectors comprise at least all of the sequences of AAV essential for encapsidation of the vector and the physical structures or inverted terminal repeats (ITRs) for infection of the recombinant AAV virions. Thus, AAV ITRs for use in the vectors of the invention need not have a wild-type nucleotide sequence (e.g., as described in Kotin, H., 1994, Gene Ther., 5:793-801), and may be altered by the insertion, deletion or substitution of nucleotides or the AAV ITRs may be derived from any of several AAV serotypes. Generally, an AAV vector is a vector derived from an adeno-associated virus serotype, including without limitation, AAV-1, AAV-2, AAV-3, AAV-4, AAV-5, AAV-6, AAV-7, AAV-8, etc. Preferred rAAV vectors have the wild type REP and CAP genes deleted in whole or part, but retain functional flanking ITR sequences that are necessary for the vector to be efficiently encapsidated by the AAV capsid to form the rAAV virion. (For example, see U.S. Pat. Nos. 7,498,024 B2 and 7,709,224 B2 expressly incorporated by reference herein in their entirety)

Among the most widely used platforms for producing rAAV involves transfecting Human Embryonic Kidney 293 (HEK293) cells with either two or three plasmids; one encoding the gene of interest, one carrying the AAV rep/cap genes, and another containing helper genes provided by either adeno or herpes viruses which contains the E4, E2a and VA helper genes that mediate AAV replication (See e.g., Janik, J. E., et. al., 1981, “Proc. Natl. Acad. Sci., 78:1925-1929; Buller, R. M. L., et al., 1981, J, Virol. vol. 40:241-24; Matsushita, T., et. al., 1998, Gene Ther., 5:938-945) The helper construct includes AAV coding regions that are capable of being expressed in the producer cell and which complement the AAV helper function that is absent in the AAV vector. Without such helper genes AAV production is generally reduced by at least a factor of 100. The helper construct may be designed to down regulate the expression of the large Rep proteins (Rep78 and Rep68), typically by mutating the start codon following p5 from ATG to ACG. (See e.g., as described in U.S. Pat. No. 6,548,286 expressly incorporated by reference herein in their entirety.) This is followed by introduction of helper virus and/or additional vectors into the producer cell, wherein the helper virus and/or additional vectors provide accessory functions capable of supporting efficient rAAV virus production. The producer cells are then cultured to produce rAAV. These steps are carried out using standard methodology. Replication-defective AAV virions encapsulating the recombinant AAV vectors of the instant invention are made by Standard techniques known in the art using AAV packaging cells and packaging technology. Examples of these methods may be found, for example, in U.S. Pat. Nos. 5,436,146, 6,040,183, 6,093,570 and 6,548,286, expressly incorporated by reference herein in their entirety. Further compositions and methods for packaging are described in Wang et al. (US 2002/0168342), expressly incorporated by reference herein in its entirety. In practicing the invention, host cells for producing rAAV virions include mammalian cells, insect cells, microorganisms and yeast. HEK293 is a commonly used host cell that has seen significant success and has become among the most preferred host cells of choice for AAV production. Host cells can also be packaging cells in which the AAV rep and cap genes are stably maintained in the host cell or producer cells in which the AAV vector genome is stably maintained and packaged. AAV vectors are purified and formulated using techniques known to those familiar in the art.

Lentiviral Vectors and Lentiviral Design for B-cell Targeting and Potentially other Cell Type Targeting

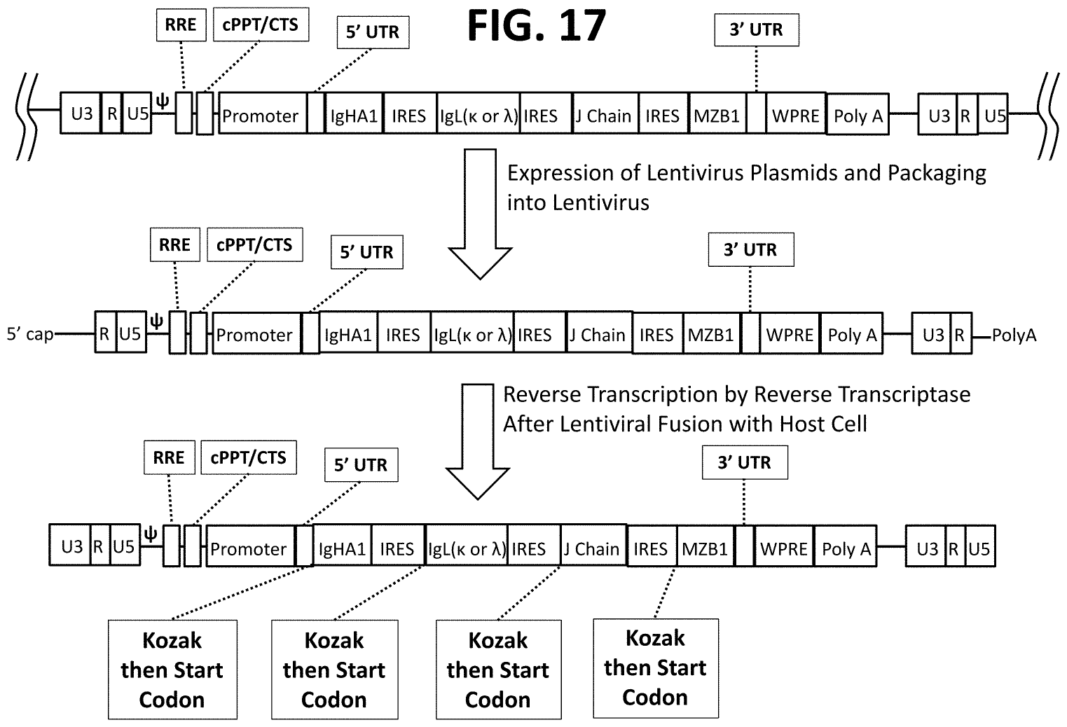

Lentiviral vectors have been extensively investigated and optimized over the past years (See e.g., Milone, M. C., et al., 2017, Leukemia, 32:1529-1541). Lentiviral vectors may be used in practicing the present invention (See FIGS. 17, 18, 19, 20, 21, 24 (vector) and 26 (vector) as examples). It should be noted that while FIGS. 17, 18, 19, 20, 21, 24 (vector) and 26 (vector) depict the dIgA genes encoded for using the coding strand for the LTRs then genes could also be incorporated into the vector using the template strand for the LTRs meaning that transcription of the dIgA1 mRNA would begin from the end closest to the 3′LTR in the direction of the 5′ LTR. In some cell types and even dependent on promoter selection this may result in a higher yield of the dIgA1 immunoglobulin and in other cells types it may result in a lower yield of the dIgA1 immunoglobulin. The relative yields can also differ based on the differentiation state of the cell. This likely has to do with the accessibility of the promoter or accessibility of the matrix associations regions (MAR) if used. Retroviral vectors have been tested and found to be suitable delivery vehicles of genes of interest into the genome of a broad range of target cells. Modified forms of lentiviral vectors may deliver episomes or extrachromosomal elements known as integration-deficient lentiviral vectors that are viral vectors may be used in the invention in one embodiment (See e.g., Wanisch, K., et al., 2009, The journal of the American Society of Gene Therapy, 17:1316-1332.). One major advantage of integration-deficient and integration competent lentiviral vectors over AAV vectors is their packaging capacity. The total packaging capacity of an AAV vector is 4.9 kilobases or about 4.6 kilobases for the transgenes and cis-acting signals after subtracting the ITRs. However, in the instant invention there are vector constructs such as related to the expression of dIgA that involve 3 or 4 transgenes including the immunoglobulin light and heavy chain as well as the J chain protein and optionally MZB1. With 4 transgenes one may not consider the use of a separate promoter for each gene if an AAV capsid is considered as the delivery vehicle. This instant invention contemplates the expression of dIgA with use of a single open reading frame that can be achieved in the tight space of the AAV viral vector. Alternatively, this instant invention contemplates the use of an integration-deficient lentivirus in one embodiment and in another embodiment to deliver the retroviral vector or the use of an integration-competent lentivirus to integrate the vector into the host genomic DNA with use of a single open reading frame and potentially a separate promoter for each transgene in some embodiments. In one embodiment two separate promoters are used to express two or three genes in a lentiviral vector such than the maximum capacity of the lentiviral vector is utilized to maximally separate the polyadenylation of the first encoded transgene and the 5′ UTR of the second encoded transgene. The packaging capacity of the integration-deficient lentiviral vector dedicated to the transgenes and transgene specific cis-acting signals is roughly about 8 kilobases.

This patent further contemplates the use of pseudotyped lentivirus to efficiently transduce targeted cells. Lentiviruses have been shown to transduce B-cells of a murine model with persistent expression of the transduced gene. This was accomplished by generating a lentivirus pseudotyped with an anti-CD19 antibody. The lentivirus anti-CD19 antibody targeted the B-cell surface receptor CD19 that mediated viral fusion upon binding the lentivirus to the B-cell, which permitted persistent expression of the transduced gene. (See e.g., Cascalho, M., et al., 2018, Sci Rep, 8:11143). Additionally, B-cells have been effectively targeted with a lentivirus pseudotyped with an envelope glycoprotein derived from the Baboon endogenous virus. (See, e.g., Fusil, F., Cosset, F. L. et. al., 2015, Molecular therapy: the journal of the American Society of Gene Therapy, 23:1734-1747) Lentivirus have seen use in clinical trials and has received FDA approval for immune based gene therapies. (See e.g., Milone, M. C., et al., 2017, Leukemia, 32:1529-1541; Campochiaro, P. A., et al., 2017, Hum Gene Ther. 28: 99-111; Milani, M., et al. 2017, EMBO Mol Med. 9:1558-73). The ability to direct the delivery of lentivirus vectors encoding one or more target protein coding sequences to specific target cells is desirable in practice of the present invention. Pesudotyping Lentivirus is well known to those familiar with the art. In all lentivirus packaging system the envelope protein or alternatively the pseudotyped protein is encoded for in a separate plasmid under the control of a CMV promoter. If the pseudotyped protein involves two separate proteins such as an immunoglobulin two separate promoters are typically used in the plasmid. This instant patent contemplates the use of pseudotyped integration deficient and also integration competent lentivirus to integrate the vector encoding for an immunoglobulin including dIgA1 and dIgA2 into genomic DNA. In one embodiment (See FIG. 24) the memory B-cell (CD 27+ receptor), naïve B-cell (CD20+ receptor or CD19+ receptor) or (CD38+ receptor or CD138+ receptor) plasma memory B-cells will have present on its surface both the endogenous heavy and light chain immunoglobulins as a B-cell receptor as well as the endogenous heavy chain in a heterodimer with the genome integrated lentiviral vector encoded light chain immunoglobulin as a B-cell receptor. Thus, having the gene therapy encoded immunoglobulin light chain presented on the naïve B-cells or memory B-cell will increase the probability that it will be activated from the target of interest. Where upon activation of memory B-cell they will give rise to either a long-lived plasma secreting cell or plasma secreting cell. The plasma secreting cell and the long-lived plasma secreting cell would produce the vector encoded dIgA1 antibody specific to the target of interest at a much higher level than the naturally encoded immunoglobulin due to a strong promoter in the lentivirus integrated vector vs. endogenous or naturally encoded Ig. The production of the long-lived plasma cell has the added benefit of producing B-cells that can persist for decades. Additionally, in other embodiments the use of pseudotyped lentivirus for delivering the vector targets other cell populations.

The light chain is ideally suited for antigen recognition as it tends to play a dominant role in target binding. Thus, the light chain binding the target will be sufficient in most cases to induce the memory B-cell to be activated and differentiate into a memory plasma B-cell or a Germinal center memory B-cell. (See, e.g. Sun, M., Li, L., Sheng Gao, Q., Pad S., 1994, The Journal of Biological Chemistry, 269:734-738; Also see, Hadzidimitriou, A., Darzentas, N., Belessi, C., et. al., 2009, Blood, 113:403-411). Other B-cells that express CD27+ markers would also be targeted including memory plasma B-cells that can survive for decades. If creating a gene therapy for an HIV immunization incorporating the HGN194 recombinant isotype of dIgA1 into a lentiviral vector construct in one embodiment one may consider to first administer the anti-CD20+ pseudotyped lentivirus carrying the lentiviral vector gene therapy to B-cells with a CD20+ marker. This can be followed by a future exposure to the HIV envelope glycoprotein such as through an mRNA based vaccine some period later that will activate the CD20+B-cells containing the dIgA1 vector that would include immature B-cells and memory B-cells that received the integration-competent or optionally integration-deficient lentiviral vector. This will result in long-term persistence of memory plasma B-cells as well as memory B-cells and Germinal Center Cells many that will encode for HGN194 recombinant isotype of dIgA1 in addition to other B-cells specific to other regions of the HIV glycoprotein that resulted from the mRNA administration of the HIV glycoprotein. Vaccine boosters of the envelope glycoprotein can increase the count of memory plasma B-cells and T follicular helper T-cells would be appropriately developed to aid in the formation of memory B-cell from Germinal Center B-cells. A similar strategy could be used in an H. pylori vaccination as well as for other lethal viruses and pathogens. Because memory plasma B-cells both migrate to the bone marrow they can provide an extra layer of protection potentially for decades if not a lifetime. (See, e.g. Akkaya, M., Kwak, K. & Pierce, S. K., 2020, Nat Rev Immunol., 20:229-238; Also see, Landsverk, 0. J., Jahnsen, F. L, et al., 2017, J. Exp. Med. 214:309-317; Khodadadi, L., Cheng, Q., Radbruch, A., & Hiepe, F., 2019, Frontiers in immunology, 10:721.) In another embodiment an anti CD27+ pseudotyped lentivirus (see FIG. 24) is used to target memory B-cells that would express both the naturally encoded heavy chain and vector encoded light chain as a B-cell receptor. Upon administration of the antigen the light chain binding the antigen will most cases be sufficient to activate the B-cell to differentiate into a memory plasma B-cell. This can also be an effective strategy to develop decades long immunity against HIV and other dangerous viruses.

The present invention provides integration-deficient replication-incompetent self-inactivating system, integration-competent replication-incompetent self-inactivating system and integration-competent replication incompetent systems where lentiviral vectors comprising one or more transgene sequences and lentiviral packaging vectors comprising one or more packaging elements. Additionally, the present invention provides pseudotyped lentiviral vectors encoding a heterologous or functionally modified envelope protein for producing pseudotyped lentivirus.

The term “vector” can refer to a ribonucleic acid (RNA) or deoxyribonucleic acid (DNA) molecule capable transferring or transporting another nucleic acid molecule. The transferred nucleic acid is generally linked to, e.g., inserted into, the vector nucleic acid molecule. A vector may include sequences that direct autonomous replication in a cell. An RNA vector is packaged into the lentivirus packaging system and is reverse transcribed into DNA following viral fusion with the transduced cell.

As is evident to one of skill in the art, the term “retroviral vector” is widely used to refer either to a nucleic acid molecule (e.g., a transfer plasmid) that includes virus-derived nucleic acid elements that typically facilitate transfer of the nucleic acid molecule by integration into the genome of a cell or to a viral particle that mediates nucleic acid retroviral transfer. Retroviral particles will typically include various viral components and sometimes also host cell components in addition to nucleic acid(s).

Once the virus is integrated into the host genome, it is referred to as a “provirus.”, The provirus serves as a template for RNA polymerase II and directs the expression of RNA molecules, which encode the structural proteins and enzymes needed to produce new viral particles. At each end of the provirus are structures called “long terminal repeats” or “LTRs.”, The term “long terminal repeat (LTR)” refers to domains of base pairs located at the ends of lentiviral vector which, in their natural sequence context, are direct repeats and contain U3, R and U5 regions. LTRs generally provide functions fundamental to the expression of lentiviral genes (e.g., promotion, initiation and polyadenylation of gene transcripts) and to viral replication. The LTR contains numerous regulatory signals including transcriptional control elements, polyadenylation signals and sequences needed for replication and integration of the viral genome. The viral LTR is divided into three regions called U3, R and U5. The U3 region contains the enhancer and promoter elements. The U5 region is the sequence between the primer-binding site and the R region and contains the polyadenylation sequence. The R (repeat) region is flanked by the U3 and U5 regions. Typically, the LTR composed of U3, R and U5 regions and appears at both the 5′ and 3′ ends of the viral genome where in the third generation system the 5′ LTR of the RNA vector has the U3 missing.

The term “packaging signal” or “packaging sequence” refers to sequences located within the lentiviral genome which are required for insertion of the viral RNA into the viral capsid or particle, (See e.g., Clever et al., 1995. J. of Virology, Vol. 69, No. 4; pp. 2101-2109.) Many lentiviral vectors use the minimal packaging signal (referred to as psi (Ψ) or (Ψ+) sequence) needed for encapsidation of the viral genome. Thus, as used in this instant patent, the terms “psi” and the symbol “Ψ′,” are used in reference to the non-coding sequence required for encapsidation of retroviral RNA strands during viral particle formation. Although, in some embodiments additional nucleotides are used at both the 5′ and 3′ ends of the psi signal to maximize efficiency of the lentivirus. “Self-inactivating” vectors refers to replication-defective vectors, e.g., lentiviral vectors, in which the 3′ LTR enhancer-promoter region, known as the U3 region, has been modified (e.g., by deletion and/or substitution) to prevent viral transcription beyond the first round of viral replication this is also a characteristic of the third generation lentiviral packaging system. This is because the 3′ LTR U3 region is used as a template for the left 5′ LTR U3 region during viral replication and, thus, the viral transcript cannot be made without the U3 enhancer-promoter. Thus the U3 of the 5′ LTR is inactivated. To render a lentiviral vector replication deficient a large part of the U3 region of the 3′LTR is deleted or modified which eliminates the viral promoter activity and also allowing for the transgene expression to be controlled by the incorporation of an internal promoter. Additionally, this large deletion of the U3 region has the added benefit of increasing transgene expression. (For example, see e.g., U.S. Pat. No. 2013/0004471 paragraph 82). The U3 region of the 5′ LTR may be replaced with a heterologous promoter to drive transcription of the viral genome during production of viral particles. Examples of heterologous promoters, which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system.

In some embodiments for maximum biosafety the 3^{rd} Generation lentiviral packaging systems are preferred. The replication-incompetent 3rd generation lentiviral packaging system splits the viral genome into 4 plasmids: (A) a plasmid containing only the genes necessary for packaging the lentiviral vector which are gag and pol which are operatively linked to a promoter, a rev gene and a polyA site (B) A plasmid that contains only the regulatory gene rev that is operatively linked to a promoter and a polyA site. (C) A plasmid that only contains the envelope gene known as env operatively linked to a promoter and a polyA site. (D) The transgene plasmid that contains the genes whose expression is desired as part of gene therapy. The transgene plasmid also contains modified LTRs at the 5′ and 3′ ends. The LTR includes a deleted or inactivated U3. Also in the LTR are the R and U5 cis-acting signals. The deleted or inactivated U3 enhancer/promoter (known as ΔU3) of the 5′ LTR is replaced with a human cytomegaloviruss (CMV) immediate early enhancer and promoter to eliminate the need for the transcriptional activator tat that has been deleted or inactivated. The viral RNA is stored in the lentivirus delivery system with a 5′LTR as a 5′-cap-R-U5 and the 3′LTR contains the mutated or deleted U3 (ΔU3) and R followed by a poly A tail of about 200 adenosines. That is the 5′ U3 is missing and the 3′ U5 is missing. The provirus that results following reverse transcription in the third generation lentiviral packaging systems contains the ΔU3-R-U5 sequences at the 5′ end and U5-R-ΔU3 sequences at the 3′ end. The first and second generation lentivirus packaging system also store the viral RNA with a 5′LTR as 5′-cap-R-U5 and the 3′LTR as U3-R followed by about 200 adenosines. The provirus that results following reverse transcription in the first and second generation lentiviral packaging systems contains the U3-R-U5 sequences at the 5′ end and U5-R-U3 sequences at the 3′ end. Additional details that describe the transgene plasmid and LTRs are described in later embodiments. The third generation lentiviral packaging system typically employs a heterologous or functionally modified envelope protein for safety. The third generation lentiviral packaging system builds on the first generation packaging system by additionally deleting the accessory genes vif, vpr, vpu and nef. Integration-deficient lentiviral vectors refer to lentiviral vectors that cannot act as retroviruses. They are viral vectors that are episomally-maintained. Both the U5 and U3 regions may be mutated at integrase (IN) attachment (att) sites to eliminate their retroviral function. This occurs through mutating specific recognition sequences necessary for integrase (IN) to attach to the vector and also cut by IN. A common strategy used to generate integration deficient lentiviral vectors is to mutate specific amino acid residues on the integrase, which is found on the pol gene, that are necessary for retroviral function. Such mutations often include either D64 or D116 of the catalytic triad. Mutating RRK (262-264) motif at the C-terminal domain causes IN to fail to bind target genomic DNA. Mutating Lysines at integrase protein positions 264, 266 and 273 impairs target DNA binding and stand transfer. These lysines residues may be acetylated in the target cell where the acetylation is required for strand transfer. Other mutations might occur at N120 or W235 that is necessary for DNA binding and thus either of these mutations block integration. If the gene encoding for the integrase protein is not mutated then the lentivirus would be integration competent and would be expected to integrate the vector into the host genomic DNA, that is also contemplated in some embodiments in this instant patent. The lentivirus (See e.g. Banasik, M. B., McCray, P. B. Jr., 2010, Gene Ther., 17:150-7. See e.g., Apolonia, L., 2009, Ph.D. Thesis, University College London; Yáñez-Muñoz, R. J., et al., 2006, Nat Med. 12:348-353; Conese, M., et. al., 2004, Gene Ther., 11:1735-1741; Karwacz, K., et al., 2009, J. Virol., 83:3094-3103).

U.S. PATENT DOCUMENTS

| | |
|---|---|
| 5,436,146 A | July 1995 Shenk et al. |
| 6,040,183 A | March 2000 Ferrari et al. |
| 6,093,570 A | July 2000 Ferrarietal et al |
| 6,548,286 B1 | April 2003 Samulski et al. |
| 7,498,024 B2 | March 2009 Fang et al. |
| 7,709,224 B2 | May 2010 Fang et al. |
| 10,251,377 B2 | April 2019 Clube et al. |
| US 2002/0168342 | November 2002 Wang et al. |
| WO 2017/005923 | January 2017 Fusil et al. |

### REFERENCED PUBLICATIONS

Lu, R M., Hwang, Y C., Liu, U. et al. Development of therapeutic antibodies for the treatment of diseases. J Biomed Sci 27, 1 (2020). https://doi.org/10.1186/s12929-019-0592-z
Iwasaki, A. & Yang, Y. “The potential danger of suboptimal antibody responses in COVID-19.”, Nat. Rev. Immunol., vol. 20, pp. 339-341, 2020. https://doi.org/10.1038/s41577-020-0321-6
Pandey, S., & Vyas, G. N. “Adverse effects of plasma transfusion.”, Transfusion, vol. 52, supp. 1 (Suppl 1), pp. 65S-79S, 2012. https://doi.org/10.1111/j1537-2995.2012.03663.x
Boe, D. M., Boule, L. A., & Kovacs, E. J. “Innate immune responses in the ageing lung.”, Clinical and experimental immunology, vol. 187, No. 1, pp. 16-25, 2017. https://doi.org/10.1016/cei.12881
Vale, A. M., & Schroeder, H. W., Jr. “Clinical consequences of defects in B-cell development.”, The Journal of allergy and clinical immunology, vol. 125, No. 4, 778-787, 2010. https://doi.org/10.1016/j.jaci.2010.02.018
Pilette, C., Ouadrhiri, Y., Sibille, Y., et al. “Lung mucosal immunity: immunoglobulin-A revisited.”, European Respiratory Journal, vol. 18, No. 3, pp. 571-588, 2001. https://erj.ersjournals.com/content/18/3/571
Terauchi, Y., Sano, K., Hasegawa, H., et al. “IgA polymerization contributes to efficient virus neutralization on human upper respiratory mucosa after intranasal inactivated influenza vaccine administration.”, Human vaccines & immunotherapeutics, vol. 14, No. 6, pp. 1351-1361, 2018. https://doi.org/10.1080/21645515.2018.1438791
Bakema, J., van Egmond, M. “The human immunoglobulin A Fc receptor FcαRI: a multifaceted regulator of mucosal immunity.” Mucosal Immunol vol 4, pp. 612-624, 2011. https://doi.org/10.1038/mi.2011.36
Melamed, D., Benschop, R. J., Cambier, J. C., & Nemazee, D., “Developmental regulation of B lymphocyte immune tolerance compartmentalizes clonal selection from receptor selection.”, Cell., vol. 92, No. 2, pp. 173-182, 1998. https://doi.org/10.1016/s0092-8674(00)80912-5
Akkaya, M., Kwak, K. & Pierce, S. K. “B cell memory: building two walls of protection against pathogens.” Nat Rev Immunol., vol. 20, pp. 229-238, 2020. https://doi.org/10.10381/s41577-019-0244-
Ziegner, M., Steinhauser, G., & Berek, C., “Development of antibody diversity in single germinal centers: selective expansion of high-affinity variants.”, Eur. J. Immunol. Vol. 24, pp. 2393-2400, 1994.
Shlomchik, M. J., Litwin, S., & Weigert, M., “The Influence of Somatic Mutation on Clonal Expansion.”, Prog. in Immunol. Proc. 7^{th} Int. Cong. Immunol. Vol. 7, pp. 415-423, 1990. https://doi.org/10.1007/978-3-642-83755-5_55
Berek, C., & Milstein, C., “Mutation drift and repertoire shift in the maturation of the immune response.”, Immunol Rev. vol. 96, pp. 23-41, 1987. doi:10.1111/j.1600-065x.1987.tb00507.x
Tiller, T., Tsuiji, M., Wardemann, H., et al. “Autoreactivity in human IgG+ memory B cells.”, Immunity, vol. 26, No. 2, pp. 205-213, 2007. https://doi.org/10.1016/j.immuni.2007.01.009
Fsuiji, M., Yurasov, S., Wardemann, H., et al. “A checkpoint for autoreactivity in human IgM+ memory B cell development.”, The Journal of Experimental Medicine vol. 203, No. 2, pp. 393-400, 2006. doi:10.1084/jem.20052033
Wardemann, H., Yurasov, S., Nussenzweig, M. C., et al. “Predominant autoantibody production by early human B cell precursors.”, Science vol. 30, pp. 1374-1377, 2003. doi:10.1126/science.1086907
Cao, Y., Su, B., Geng, C., et al. “Potent neutralizing antibodies against SARS-CoV-2 identified by high-throughput single-cell sequencing of convalescent patients' B cells.”, Cell, vol. 182, pp. 73-84, 2020. https://doi.org/10.1016/j.cell.2020.05.025.
Weiss, G. E., Ndungu, F. M., & Pierce, S. K. “High efficiency human memory B cell assay and its application to studying Plasmodium falciparum-specific memory B cells in natural infections.”, Journal of immunological methods, vol. 375, No. 1-2, pp. 68-74. 2012. https://doi.org/10.1016/j.jim.2011.09.006
Hunter, S. A., & Cochran, J. R., “Cell-Binding Assays for Determining the Affinity of Protein-Protein Interactions: Technologies and Considerations.”, Methods Enzymol. vol. 580, pp. 21-44, 2016. doi:10.1016/bs.mie.2016.05.002
Li, P., Selvaraj, P., & Zhu, C. “Analysis of competition binding between soluble and membrane-bound ligands for cell surface receptors.”, Biophysical journal, vol. 77, No. 6, pp. 3394-3406, 1999. https://doi.org/10.1016/S0006-3495(99)77171-7
Amanna, I. J., & Slifka, M. K., “Quantitation of rare memory B cell populations by two independent and complementary approaches.”, Journal of immunological methods, vol. 317, No. 1-2, pp. 175-185, 2006. https://doi.org/10.1016/j.jim.2006.09.005
Shen S, et al. (2007) Expression, glycosylation, and modification of the spike (S) glycoprotein of SARS CoV. Methods Mol Biol. 379: 127-35.
Du L, et al. (2009) The spike protein of SARS-CoV—a target for vaccine and therapeutic development. Nat Rev Microbiol. 7 (3): 226-36.
Xiao X, et al. (2004) The SARS-CoV S glycoprotein. Cell Mol Life Sci. 61 (19-20): 2428-3
Setliff, I., Shiakolas, A. R Ronsard, L., et al., “High-Throughput Mapping of B Cell Receptor Sequences to Antigen Specificity.” Cell, vol. 179, pp. 1636-1646, 2019.
Wu, X., Yang, Z. Y., Kwong, P. D., et al. “Rational design of envelope identifies broadly neutralizing human monoclonal antibodies to HIV-1.” Science, vol. 3295, pp. 856-861, 2010. https:/doi.org/10.1126science.1187659
Niu, X., Zhao, L., Zhang, X., et al. Chen, L. (2019). Convalescent patient-derived monoclonal antibodies targeting different epitopes of E protein confer protection against Zika virus in a neonatal mouse model. Emerging microbes & infections, 8(1), 749-759. https://doi.org/10.1080/22221751.2019.1614885
Crotty, S., Aubert, R. D., Glidewell, J., & Ahmed, R. “Tracking human antigen-specific memory B cells: a sensitive and generalized ELISPOT system.”, J Immunol Methods., vol. 286, No. 1-2, pp. 111-122, 2004. doi:10.1016/j.jim.2003.12.015
Antonio, E., Muruato, C. R., Fontes-Garfias, P. R., Mariano G.-B., Vineet, D., Menachery, X. X., Pei-Yong, S. “A high-throughput neutralizing antibody assay for COVID-19 diagnosis and vaccine evaluation” bioRxiv 2020.05.21.109546; doi: https://doi.org/10.1101/2020.05.21.109546
Robbiani, D. F., Gaebler, C., Muecksch, F. et al. Convergent antibody responses to SARS-CoV-2 in convalescent individuals. Nature vol. 584, pp. 437-442 (2020). https://doi.org/10.1038/s41586-020-2456-9
Sarzotti-Kelsoe, M., Daniell, X., Montefiori, D. C., et al. “Optimization and validation of a neutralizing antibody assay for HIV-1 in A3R5 cells.”, Journal of immunological methods, vol. 409, pp. 147-160, 2014. https:./doi.org/10.1016/j.jim.2014.02.013
Klein, U., Rajewsky, K., Kuppers, R., “Human immunoglobulin (Ig)M+ IgD+ peripheral blood B cells expressing the CD27 cell surface antigen carry somatically mutated variable region genes: CD27 as a general marker for somatically mutated (memory) B cells.”, The Journal of Experimental Medicine vol. 188, pp. 1679-1689, 1998.
Vale, A. M., Cavazzoni, C. B., Nobrega, A., & Schroeder, H. W. “The Global Self-Reactivity Profile of the Natural Antibody Repertoire Is Largely Independent of Germline DH_{H} Sequence.”, Front. Immunol. vol. 7, No. 296., 2016. doi: 10.3389/fimmu.2016.00296
Nobrega, A., Haury, M, Coutinho, A. et al. “Global analysis of antibody repertoires. II. Evidence for specificity, self-selection and the immunological “homunculus” of antibodies in normal serum.”, Eur J Immunol., vol. 23, No. 11, pp. 2851-2859, 1993. doi:10.1002/eji.1830231119
Haury, M., Grandien, A., Sundblad, A., Coutinho, A., & Nobrega, A. “Global analysis of antibody repertoires. 1. An immunoblot method for the quantitative screening of a large number of reactivities.”, Scand. J. Immunol. vol. 39, No. 1, pp. 79-87, 1994. doi:10.1111/j.1365-3083.1994.tb03343.x
Lu, J., Chu, J., Zou, Z., Hamacher, N. B., Rixon, M. W., & Sun, P. D. (2015). Structure of FcγRI in complex with Fc reveals the importance of glycan recognition for high-affinity IgG binding. Proceedings of the National Academy of Sciences of the United States of America, 112(3), 833-838. https://doi.org/10.1073/pnas 1418812112
Pritsch, O., Hudry-Clergeon, G., Buckle, M., et al. “Can immunoglobulin C(H)1 constant region domain modulate antigen binding affinity of antibodies?.”, J Clin Invest. Vol. 98. No. 10, pp. 2235-2243, 1996. doi:10.1172/JCI119033
Janda, A., Bowen, A., Greenspan, N. S., Casadevall, A. “Ig Constant Region Effects on Variable Region Structure and Function.”, Front Microbiol. vol. 7 No. 22, 2016. Published 2016 Feb. 4. doi:10.3389/fmicb.2016.00022
Kumar Bharathkar, S., Parker, B. W., Malyutin, A., Haloi, N., Tajkhorshid, E., Stadtmueller, B. M. “The Structures of Secretory and Dimeric Immunoglobulin A” bioRxiv 2020.02.16.951780; doi: https://doi.org/10.1101/2020.02.16.951780
Toraño, A., & Putnam, F. W., “Complete amino acid sequence of the alpha 2 heavy chain of a human IgA2 immunoglobulin of the A2m (2) allotype.” Proceedings of the National Academy of Sciences of the United States of America, vol. 75, pp. 966-969, 1978 https://doi.org/10.1073/pnas.75.2.966;
Li, Z., “The forgotten tale of immunoglobulin allotypes in cancer risk and treatment.” Exp Hematol Oncol 2, 6, 2013.
Fang, J., Qian, J., Yi, S. et al. “Stable antibody expression at therapeutic levels using the 2A peptide.”, Nat. Biotechnol., vol. 23, pp. 584-590, 2005. https://doi.org/10.1038/nbt1087
Krysan, D. J., Rockwell, N. C., Fuller, R. S. “Quantitative characterization of furin specificity. Energetics of substrate discrimination using an internally consistent set of hexapeptidyl methylcoumarinamides.”, J Biol Chem. vol. 274, No. 33, pp. 23229-23234, 1999. doi:10.1074/jbc.274.33.23229
Lewis, A. D., Chen, R., Montefiori, D. C., Johnson. P. R., Clark, K. R. “Generation of neutralizing activity against human immunodeficiency virus type 1 in serum by antibody gene transfer.”, J Virol. vol. 76, No. 17, pp. 8769-8775, 2002. doi:10.1128/jvi.76.17.8769-8775.2002
Huang, M. T., & Gorman, C. M. “The simian virus 40 small-t intron, present in many common expression vectors, leads to aberrant splicing.”, Molecular and cellular biology, vol. 10, No. 4, pp. 1805-1810, 1990. https.//doi.org/10.1128/mcb. 10.4.1805
Wu, C., & Alwine, J. C. “Secondary structure as a functional feature in the downstream region of mammalian polyadenylation signals.”, Molecular and cellular biology, vol. 24, No. 7, pp. 2789-2796, 2004. https://doi.org/10.1128/mcb.24.7.2789-2796.2004
Lycke, N., Erlandsson, L., Ekman, L., Schön, K., Leanderson, T. “Lack of J chain inhibits the transport of gut IgA and abrogates the development of intestinal antitoxic protection.”, J. Immunol., vol. 163, pp. 913-919, 1999.
Sørenson V., Sundvold, V., Michaelsen, T. E., Sandlie, I. “Polymerization of IgA and IgM: Roles of Cys309/Cys414 and the secretory tailpiece.”, J. Immunol., vol. 162, pp. 3448-3455, 1999.
Schroeder, H., Wald, D., Greenspan, N., “Chapter 4: Immunoglobulins: Structure and Function”. In Paul, William (ed.). Fundamental Immunology (Book) (6th ed.). Philadelphia, Pa.: Lippincott Williams & Wilkins. pp. 125-151, 2008. ISBN 978-0-7817-6519-0.
Stadtmueller, B. M., Huey-Tubman, K. E., Bjorkman, P. J., et al. “The structure and dynamics of secretory component and its interactions with polymeric immunoglobulins.”, Elife. vol. 5, e10640, 2016. Published 2016 Mar. 4. doi:10.7554/eLife.10640
Castro, C. D., & Flajnik, M. F., “Putting J chain back on the map: how might its expression define plasma cell development?” Journal of Immunology vol. 193, No. 7, pp. 3248-3255, 2014. https://doi.org/10.4049/jimmunol.1400531
Koshland, M. E., “The coming of age of the immunoglobulin J chain.”, Annu Rev Immunol. vol. 3, pp. 425-453, 1985. doi:10.1146/annurev.iy.03.040185.002233
Bonner, A., Almogren, A., Furtado, P. et al. “Location of secretory component on the Fc edge of dimeric IgA1 reveals insight into the role of secretory IgA1 in mucosal immunity.”, Mucosal Immunol. vol. 2, pp. 74-84, 2009. https://doi.org/10.1038/mi.2008.68
Johansen, F. E., Braathen, R., Brandtzaeg, P. “Role of J Chain in Secretory Immunoglobulin Formation”. Scandinavian Journal of Immunology. vol. 52, No. 3, pp. 240-248, 2000. doi:10.1046/j.1365-3083.2000.00790.x. PMID 10972899
Wang, Y., Wang, G., Li, Y. et al. “Structural insights into secretory immunoglobulin A and its interaction with a pneumococcal adhesin.” Cell Res, vol. 30, pp. 602-609, 2020. https://doi.org/10.1038/s41422-020-0336-3
Xiong, E., Li, Y., Wang, J. Y., et al. “MZB1 promotes the secretion of J chain-containing dimeric IgA and is critical for the suppression of gut inflammation.”, Proceedings of the National Academy of Sciences of the United States of America, vol. 116, No. 27, pp. 13480-13489, 2019. https://doi.org/10.1073/pnas.1904204116
Mak, T. W., Saunders, M. E., Jett, B. D., Primer to the Immune Response (Second Edition), Academic Cell, 2014, pp. 269-292, ISBN 9780123852458, https://doi.org/10.1016/B978-0-12-385245-8.00012-1
Ben Mkaddem, S., Benhamou, M., & Monteiro, R. C., “Understanding Fc Receptor Involvement in Inflammatory Diseases: From Mechanisms to New Therapeutic Tools.” Frontiers in immunology, vol. 10, pp. 811, 2019. https://doi.org/10.3389/fimmu.2019.00811
O'Brien, C., Flower, D. R., & Feighery, C., “Peptide length significantly influences in vitro affinity for MHC class II molecules.” Immunome research, 4, 6, 2008. https://doi.org/10.1186/1745-7580-4-6
Gehring, A. J., Sun, D., Bertoletti, A., et al. “The level of viral antigen presented by hepatocytes influences CD8 T-cell function.” J Virol., vol. 81, pp. 2940-9, 2007. https://doi.10.1128/JVI.02415-06
Arber, C., Abhyankar, H., Heslop, H. E., Brenner, M. K., Liu, H., Dotti, G., & Savoldo, B. (2013). “The immunogenicity of virus-derived 2A sequences in immunocompetent individuals.” Gene therapy, vol. 20, pp. 958-962. https://doi.org/10.1038/gt2013.25
Nagaraju, K., Raben, N., Plotz, P., et al. “Conditional up-regulation of MHC class I in skeletal muscle leads to self-sustaining autoimmune myositis and myositis-specific autoantibodies.” Proceedings of the National Academy of Sciences of the United States of America, vol. 97, pp. 9209-9214. https://doi.org/10.1073/pnas.97.16.9209
Sips, M., Krykbaeva, M., Alter, G., et al. “Fc receptor-mediated phagocytosis in tissues as a potent mechanism for preventive and therapeutic HIV vaccine strategies.” Mucosal Immunol. vol. 9, pp. 1584-1595, 2016 doi: 10.1038/mi.2016.12.
Kruize, Z., Kootstra, N., “The Role of Macrophages in HIV Persistence and Pathogenesis” Frontiers in Microbiology, vol. 10, pp. 1-17, 2019.
Shen, R., Richter, H. E., Smith, P. D., et al. “Macrophages in vaginal but not intestinal mucosa are monocyte-like and permissive to human immunodeficiency virus type 1 infection”. Journal of virology, vol. 83, pp. 3258-3267, 2009. https://doi.org/10.1128/JVI.01796-08
Gobeil, L. A., Lodge, R., Tremblay, M. J. “Macropinocytosis-like HIV-1 internalization in macrophages is CCR5 dependent and leads to efficient but delayed degradation in endosomal compartments.” J Virol. Vol. 87, 735-45, 2013. doi: 10.1128/JVI.01802-12.
Carter, G. C., Bernstone, L., Baskaran, D., James, W. “HIV-1 infects macrophages by exploiting an endocytic route dependent on dynamin, Rac1 and Pak1.” Virology, vol. 409, pp. 234-250, 2011.
Watkins, J. D., Sholukh, A. M., Mukhtar, M. M., Siddappa, N. B., Lakhashe, S. K., Kim, M., Reinherz, E. L., Gupta, S., Forthal, D. N., Sattentau, Q. J., Villinger, F., Corti, D., Ruprecht, R. M.; CAVD Project Group. “Anti-HIV IgA isotypes: differential virion capture and inhibition of transcytosis are linked to prevention of mucosal R5 SHIV transmission.” AIDS. 2013 vol. 27, pp. F13-20, 2013.
Tomaras, G. D., Ferrari, G., Haynes, B. F. et al. “Vaccine-induced plasma IgA specific for the C1 region of the HIV-1 envelope blocks binding and effector function of IgG.” Proceedings of the National Academy of Sciences of the United States of America, vol. 110, pp. 9019-9024, 2013. https://doi.org/10.1073/pnas.1301456110
Haynes B F, et al., Immune-correlates analysis of an HIV-1 vaccine efficacy trial. N Engl J Med, vol. 366, pp. 1275-1286, 2012.
Abdullah, M., Greenfield, L. K., Bronte-Tinkew, D. et al. VacA promotes CagA accumulation in gastric epithelial cells during Helicobacter pylori infection. Sci Rep 9, 38 (2019). https://doi.org/10.1038/s41598-018-37095-4
Atherton, J. C., Cao, P., Peek, R. M. Jr, Tummuru, M. K., Blaser, M. J., Cover, T. L., “Mosaicism in vacuolating cytotoxin alleles of Helicobacter pylori. Association of specific vacA types with cytotoxin production and peptic ulceration.” J Biol Chem, Jvol. 270, pp. 17771-17777, 1995. doi: 10.1074/jbc.270.30.17771.
Kuck, D., Kolmerer, B., Iking-Konert, C., Krammer, P. H., Stremmel, W., & Rudi, J., “Vacuolating cytotoxin of Helicobacter pylori induces apoptosis in the human gastric epithelial cell line AGS.” Infection and immunity, vol. 69, pp. 5080-5087, 2001. https://doi.org/10.1128/IAI.69.8.5080-5087.2001
Palframan, S. L., Kwok, T., Gabriel, K., “Vacuolating cytotoxin A (VacA), a key toxin for Helicobacter pylori pathogenesis” Frontiers in Cellular and Infection Microbiology, v. 2, pp. 1-9, 2012. doi:10.3389/fcimb.2012.00092
Xu, C., Soyfoo, D. M., Wu, Y., & Xu, S., “Virulence of Helicobacter pylori outer membrane proteins: an updated review.” European journal of clinical microbiology & infectious diseases: official publication of the European Society of Clinical Microbiology, vol. 39, pp. 1821-1830, 2020. https://doi.org/10.1007/s10096-020-03948-y
Königer, V., Holsten, L., Harrison, U. et al. Helicobacter pylori exploits human CEACAMs via HopQ for adherence and translocation of CagA. Nat Microbiol, vol. 2, pp. 16188, 2017. https://doi.org/10.1038/nmicrobiol.2016.188
Bonsor, D. A., Zhao, Q., Sundberg, E. J., et al. The Helicobacter pylori adhesin protein HopQ exploits the dimer interface of human CEACAMs to facilitate translocation of the oncoprotein CagA. The EMBO journal, vol. 37, pp. e98664, 2018. https://doi.org/10.15252/embj.201798664
Borén, T., Falk, P., Roth, K. A., Larson, G., Normark, S., “Attachment of Helicobacter pylori to human gastric epithelium mediated by blood group antigens.” Science. Vol. 262, pp. 1892-5, 1993. doi: 10.1126/science.8018146.
Spencer, J., Sollid, L. “The human intestinal B-cell response.” Mucosal Immunol, vol. 9, pp. 1113-1124, 2016. https://doi.org/10.1038/mi.2016.59
Lycke, N., Bemark, M. “The regulation of gut mucosal IgA B-cell responses: recent developments.” Mucosal Immunol, vol. 10, pp. 1361-1374, 2017. https://doi.org/10.1038/mi.2017.62
Cifarelli, V., Eichmann, A., “The Intestinal Lymphatic System: Functions and Metabolic Implications” Cellular and Molecular Gastroenterology and Hepatology, 7, pp. 503-513, 2019
Baluk, P., Fuxe, J., McDonald, D. M., et al. “Functionally specialized junctions between endothelial cells of lymphatic vessels.” The Journal of experimental medicine, vol. 204, pp. 2349-2362, 2007. https://doi.org/10.1084/jem.20062596
Venugopal, A. M., Stewart, R. H., Laine, G. A., Dongaonkar, R. M., Quick, C. M., “Lymphangion coordination minimally affects mean flow in lymphatic vessels.” Am J Physiol Heart Circ vol. 293, pp. H1183-9, 2007. doi: 10.1152/ajpheart.01340.2006.
Fusil, F., Calattini, S., Cosset, F. L., et al. “A Lentiviral Vector Allowing Physiologically Regulated Membrane-anchored and Secreted Antibody Expression Depending on B-cell Maturation Status.” Molecular therapy: the journal of the American Society of Gene Therapy, vol. 23, pp. 1734-1747. https://doi.org/10.1038/mt.2015.148
Trepotec, Z., Aneja, M. K., Geiger, J., Hasenpusch, G., Plank, C., Rudolph, C., “Maximizing the Translational Yield of mRNA Therapeutics by Minimizing 5′-UTRs.” Tissue Eng Part A. vol 1-2, 69-79, 2019. doi: 10.1089/ten.TEA.2017.0485.
Leppek, K., Das, R. & Barna, M. “Functional 5′ UTR mRNA structures in eukaryotic translation regulation and how to find them.” Nat Rev Mol Cell Biol, vol. 19, pp. 158-174, 2018. https://doi.org/10.1038/nrm.2017.103
Calvo, S. E., Pagliarini, D. J., Mootha, V. K., “Upstream open reading frames cause widespread reduction of protein expression and are polymorphic among humans.” PNAS, v. 18, pp. 7507-7512, 2009. https://www.pnas.org/content/106/18/7507
Babendure, J. R., Babendure, J. L., Ding, J. H., & Tsien, R. Y. “Control of mammalian translation by mRNA structure near caps.” RNA, vol. 12, pp. 851-861, 2006. https://doi.org/10.1261/rna.2309906
Barrett, L. W., Fletcher, S., & Wilton, S. D., “Regulation of eukaryotic gene expression by the untranslated gene regions and other non-coding elements.” Cellular and molecular life sciences: CMLS, vol. 69, pp. 3613-3634, 2012. https://doi.org/10.1007/s00018-012-0990-9
Siepel, A., Bejerano, G., Haussler, D. et al. “Evolutionarily conserved elements in vertebrate, insect, worm, and yeast genomes.” Genome research, vol. 15, pp. 1034-1050, 2005. https://doi.org/10.1101/gr.3715005
Berkovits, B., Mayr, C. “Alternative 3UTRs act as scaffolds to regulate membrane protein localization”. Nature, vol. 522, pp. 363-367, 2015. https://doi.org/10.1038/nature14321
Kirk, S. J., Cliff, J. M., Thomas, J. A., Ward, T. H. “Biogenesis of secretory organelles during B cell differentiation.” J Leukoc Biol. Vol. 87, pp. 245-55, 2010. doi: 10.1189/jlb.1208774.
Lei, Y., Huang, T., Su, M. et al. “Expression and distribution of immunoglobulin G in the normal liver, hepatocarcinoma and postpartial hepatectomy liver.” Lab Invest vol. 94, pp. 1283-1295, 2014. https://doi.org/10.1038/labinvest.2014.114
Feldmann, G., Penaud-Laurencin, J., Crassous, J., Benhamou, J. P., “Albumin Synthesis in Liver Cells: It's Morphlogical Demonstration” Gastroenterology, vol. 63, pp. 1036-1049, 1972
Woo, D. H., Kim, S. K., Kim, J. H. et al. “Direct and indirect contribution of human embryonic stem cell-derived hepatocyte-like cells to liver repair in mice.” Gastroenterology. Vol. 142, pp. 602-11, 2012. doi: 10.1053/j.gastro.2011.11.030.
Sharma, N. S., Nagrath, D., & Yarmush, M. L. “Metabolic profiling based quantitative evaluation of hepatocellular metabolism in presence of adipocyte derived extracellular matrix.” PloS one, vol. 6, e20137, 2011. https://doi.org/10.1371/journal.pone.0020137
Reichmuth, A. M., Oberli, M. A., Jaklenec, A., Langer, R., & Blankschtein, D., “mRNA vaccine delivery using lipid nanoparticles.” Therapeutic delivery, vol. 7, 319-334, 2016. https://doi.org/10.4155/tde-2016-0006
Renz, H., Allen, K., Sicherer, S. et al. “Food allergy.” Nat Rev Dis Primers, pp. 4, vol. 17098, 2018. https://doi.org/10.1038/nrdp.2017.98
He, S. H., Zhang, H. Y., Zeng, X. N., Chen, D., & Yang, P. C., “Mast cells and basophils are essential for allergies: mechanisms of allergic inflammation and a proposed procedure for diagnosis.” Acta pharmacologica Sinica, vol. 34, pp. 1270-1283, 2013. https://doi.org/10.1038/aps.2013.88
Holgate, S., Wenzel, S., Postma, D. et al. Asthma. Nat Rev Dis Primers, vol. 1, pp. 15025, 2015. https://doi.org/10.1038/nrdp.2015.25
Choy, D. F., Bradding, P., et. al. “TH2 and TH17 inflammatory pathways are reciprocally regulated in asthma.” Sci Transl Med., vol. 7, pp. 301, 2015. doi: 10.1126/scitranslmed.aab3142. PMID: 26290411.\
Liu, J., Pang, Z., Wang, G., Guan, X., Fang, K., Wang, Z., & Wang, F. “Advanced Role of Neutrophils in Common Respiratory Diseases.” Journal of immunology research, 2017, 6710278. https://doi.org/10.1155/2017/6710278
Sun, L., Zhang, L., Yu, J. et al. “Clinical efficacy and safety of anti-PD-1/PD-L1 inhibitors for the treatment of advanced or metastatic cancer: a systematic review and meta-analysis.” Sci Rep, vol. 10, pp. 2083, 2020. https://doi.org/10.1038/s41598-020-58674-4
Zhang, X., & Chang, A., “Somatic mutations of the epidermal growth factor receptor and non-small-cell lung cancer.” Journal of medical genetics, vol. 44, pp. 166-172, 2007. https://doi.org/10.1136/jmg.2006.046102
Owens, M. B., Hill, A. D., & Hopkins, A. M., “Ductal barriers in mammary epithelium.” Tissue barriers, 1:e25933, 2013. https://doi.org/10.4161/tisb.25933
Truong, B., Allegri, G., Liu, X. B., Burke, K. E., Zhu, X., Cederbaum, S. D., Häberle, J. P., Martini, G. V., Lipshutz, G. S., “Lipid nanoparticle-targeted mRNA therapy as a treatment for the inherited metabolic liver disorder arginase deficiency” PNAS, vol. 116, pp. 21150-21159, 2019. DOI: 10.1073/pnas.1906182116
Kim, M., Jeong, M., Woo, H. A., Nam, K. T., Lee, K., Lee, H., “Engineered ionizable lipid nanoparticles for targeted delivery of RNA therapeutics into different types of cells in the liver” vol. 7, pp. 1-12, 2021. DOI: 10.1126/sciadv.abf4398
Veiga, N., Goldsmith, M., Granot, Y. et al. “Cell specific delivery of modified mRNA expressing therapeutic proteins to leukocytes.” Nat Commun, vol. 9, pp. 4493, 2018. https://doi.org/10.1038/s41467-018-06936-1
Yip, M. S. et al. “Antibody-dependent enhancement of SARS coronavirus infection and its role in the pathogenesis of SARS.”, Hong Kong Med. J. vol. 22, pp. 25-31, 2016
Wang, S. F. et al. “Antibody-dependent SARS coronavirus infection is mediated by antibodies against spike proteins.”, Biochem. Biophys. Res. Commun. vol. 451, pp. 208-214, 2014.
Yasui, F. et al. “Prior immunization with severe acute respiratory syndrome (SARS)-associated coronavirus (SARS-CoV) nucleocapsid protein causes severe pneumonia in mice infected with SARS-CoV.”, J. Immunol., vol. 181, pp. 6337-6348, 2008.
Janeway, C., Travers, P., Walport, M., Shlomchik, M., Janeway's Immunobiology, Ninth Edition. 2016. Garland Science: New York, N.Y. ISBN: (Paperback) 978-0815345053.
Stavnezer, J., & Schrader, C. E. “IgH chain class switch recombination: mechanism and regulation.”, Journal of immunology (Baltimore, Md.: 1950), vol. 193, No. 11, pp. 5370-5378, 2014. https://doi.org/10.4049/jimmunol.1401849
Eigenmann M. J., Karlsen, T. V., Wiig, H., et al. “Interstitial IgG antibody pharmacokinetics assessed by combined in vivo- and physiologically-based pharmacokinetic modelling approaches”. J Physiol. Vol. 595, pp. 7311-7330, 2017. doi: 10.1113/JP274819. Epub 2017 Oct. 29.
Borrok, M. J., DiGiandomenico, A., Beyaz, N., Marchetti, G. M., Barnes, A. S., Lekstrom, K. J., Phipps, S. S., McCarthy, M. P., Wu, H., Dall'Acqua, W. F., Tsui, P., & Gupta, R. “Enhancing IgG distribution to lung mucosal tissue improves protective effect of anti-Pseudomonas aeruginosa antibodies.” JCI insight, vol. 3, pp. e97844) https://doi.org/10.1172/jci.insight.97844
Kim, H. Y., Tsai, S., Izadjoo, M. J., et al. “Production and characterization of chimeric monoclonal antibodies against Burkholderia pseudomallei and B. mallei using the DHFR expression system.” PloS one, vol. 6, pp. e19867, 2011. https://doi.org/10.1371/journal.pone.0019867
Lee, E. C., Liang, Q., Ali, H. et al. “Complete humanization of the mouse immunoglobulin loci enables efficient therapeutic antibody discovery.” Nat Biotechnol vol. 32, pp. 356-363, 2014. https://doi.org/10.1038/nbt.2825
Tjelle, T. E., Corthay, A., Lunde, E., Sandlie, I., Michaelsen, T. E., Mathiesen, I., Bogen, B., “Monoclonal antibodies produced by muscle after plasmid injection and electroporation” Mol. Ther., vol. 9, pp. 328-336, 2004.
Lei, Y., Huang, T., Su, M. et al. Expression and distribution of immunoglobulin G in the normal liver, hepatocarcinoma and postpartial hepatectomy liver. Lab Invest vol. 94, pp. 1283-1295, 2014. https://doi.org/10.1038/labinvest.2014.114
Andrews, C. D., Luo, Y., Sun, M, Ho D. D., et al. “In Vivo Production of Monoclonal Antibodies by Gene Transfer via Electroporation Protects against Lethal Influenza and Ebola Infections Molecular Therapy”—Methods and Clinical Development, vol. 7, No. 15, pp. 74-82, 2017
Kim, et al., “Use of the Human Elongation Factor 1 O. Promoter as a Versatile and Efficient Expression System’. Gene, vol. 91, pp. 217-223, 1990.
Guo, et al., “Evaluation of Promoter Strength for Hepatic Gene Expression In Vivo Following Adenovirus-Mediated Gene Transfer”, Gene Ther, vol. 3, pp. 802-810, 1996.
Furler, et al., “Recombinant AAV Vectors Containing the Foot-and-Mouth Disease Virus 2A Sequence Confer Efficient Bicistronic Gene Expression in Cultured Cells and Rat Substantia Nigra Neurons'. Gene Ther, vol. 8, pp. 864-873, 2001.
Conese, M., Auriche, C. & Ascenzioni, F. Gene “Therapy Progress and Prospects: Episomally maintained self-replicating systems.”, Gene Ther., vol. 11, pp. 1735-1741, 2004. https://doi.org/10.1038/sj.gt.3302362
Kotin, “Prospects for the Use of Adeno-Associated Virus as a Vector for Human Gene Therapy’. Hum. Gene Ther . . . vol. 5, pp. 793-801, 1994.
Janik, J. E., Huston, M. M., Rose, J. A., “Locations of adenovirus genes required for the replication of adenovirus-associated virus.”, Proc. Natl. Acad. Sci., vol. 78 pp. 1925-1929, 1981.
Buller, R. M. L., et al. “Herpes simplex virus types 1 and 2 completely help adenovirus-associated virus replication.”, J Virol vol. 40, pp. 241-24, 1981.
Matsushita, T., Elliger, S., Elliger, C., et al. “Adeno-associated virus vectors can be efficiently produced without helper virus.”, Gene Ther., vol. 5, No. 7, pp. 938-945, 1998) doi:10.1038/sj.gt.3300680
Milone, M. C., O'Doherty, U. “Clinical use of lentiviral vectors.”, Leukemia. vol. 32, No. 7, pp. 1529-1541, 2017. doi:10.1038/s41375-018-0106-0
Wanisch, K., & Yáñez-Muñoz, R. J., “Integration-deficient lentiviral vectors: a slow coming of age. Molecular therapy.”, The journal of the American Society of Gene Therapy, vol. 17, No. 8, pp. 1316-1332, 2009. https://doi.org/10.1038/mt.2009.122
Campochiaro P. A., Lauer, A. K., Sohn, E. H., Mir, T. A., Naylor S., Anderton, M. C., et al. “Lentiviral vector gene transfer of endostatin/angiostatin for macular degeneration (GEM) study.”, Hum Gene Ther. vol. 28, pp. 99-111, 2017.
Milani M, Annoni A, Biffi M, Di Tomaso T, et al. Genome editing for scalable production of alloantigen-free lentiviral vectors for in vivo gene therapy. EMBO Mol Med. 2017; 9:1558-73.
Cascalho, M., Huynh, D., Lefferts, A. R. et al. “Durable targeting of B-lymphocytes in living mice.”, Sci Rep, vol. 8, pp. 11143, 2018. https://doi.org/10.1038/s41598-018-29452-0
Wu, Z., Asokan, A., Samulski, R. J., “Adeno-associated virus serotypes: vector toolkit for human gene therapy.”, Mol Ther. vol. 14, pp. 316-327, 2006.
Sun, M., Li, L., Sheng Gao, Q., Pad S., “Antigen Recognition by an Antibody Light Chain” The Journal of Biological Chemistry, vol. 269, pp. 734-738, 1994.
Hadzidimitriou, A., Darzentas, N., Belessi, C., et. al., “Evidence for the significant role of immunoglobulin light chains in antigen recognition and selection in chronic lymphocytic leukemia” Blood, vol. 113, pp. 403-411, 2009
Landsverk, O. J., Snir, O., Jahnsen, F. L. et al., “Antibody-secreting plasma cells persist for decades in human intestine.” The Journal of experimental medicine, vol. 214, pp. 309-317. https://doi.org/10.1084/jem.20161590
Khodadadi, L., Cheng, Q., Radbruch, A., & Hiepe, F., “The Maintenance of Memory Plasma Cells.” Frontiers in immunology, vol. 10, pp. 721. https://doi.org/10.3389/fimmu.2019.00721
Banasik, M. B., McCray, P. B. Jr. “Integrase-defective lentiviral vectors: progress and applications.” Gene Ther., vol. 17, pp. 150-7, 2010. doi: 10.1038/gt.2009.135
Apolonia, L., “Development and Application of Non-Integrating Lentiviral Vectors for Gene Therapy” Ph.D. Thesis, University College London; London, U K, 2009.
Yañez-Muñoz, R. J., Balaggan, K. S., MacNeil, A., et al. “Effective gene therapy with nonintegrating lentiviral vectors.”, Nat Med. Vol. 12, No. 3, pp. 348-353, 2006. doi:10.1038/nm1365
Karwacz, K., Mukherjee, S., Thrasher. A. J., et al. “Nonintegrating lentivector vaccines stimulate prolonged T-cell and antibody responses and are effective in tumor therapy.”, J. Virol. Vol. 83, pp. 3094-3103, 2009.

What is claimed:

1. Episomal, genomic integrated lentiviral vector or mRNA expression of monoclonal or polyclonal antibodies (immunoglobulins) of one or more of isotypes IgG1, IgG2, IgG3, IgA1, dIgA1, IgA2, or dIgA2 where the episome, genomic integrated lentiviral vector or mRNA encodes for the polypeptide sequence for immunoglobulins light and heavy chains as well as J Chain for dIgA1 and dIgA2 that are expressed in the same cell and are identified from one or more of (A) CD27+ IgG memory B-cells (B) CD27+ IgA memory B-cells, (C) any memory B-cell (D) memory plasma B-cell (E) plasma B-cell (F) plasmablasts (G) from any transgenic animal (H) from a mouse or rabbit with a humanized immunized system (I) from a mouse other non-human vertebrate antibody converted into a chimeric antibody. Where IgG and IgA memory B-cells are derived from the blood of persons or animals who are currently infected with, were previously infected, were previously exposed to, has immune specificity to, or are affected by one or more of (1) a virus (s) (2) a systemic ailment such as allergies (3) Allergens (4) fungi (5) bacterial infection (6) cancerous tumor (7) an unnatural virus or toxin (8) microbial infection, (9) any ailment (10) a target protein or variant including self-antigens

2. An mRNA, viral, non-viral or retroviral vector coding for one or more of dimeric immunoglobulin A1 (dIgA1) and/or dIgA2 where the vector contains the transgenes in any order for (1) the immunoglobulin heavy chain of isotype A1 (IgHA1), the immunoglobulin light chain that may be kappa (IgLκ) or lambda (IgLλ) as determined from gene sequencing of the B-cell of interest in claim 1 and J chain or (2) the immunoglobulin heavy chain of isotype A1 (IgHA1), the immunoglobulin light chain that may be kappa (IgLκ) or lambda (IgLλ) as determined from gene sequencing of the B-cell of interest in claim 1, J chain and MZB1. Where the immunoglobulin light and heavy chains encoded for in any nucleic acid vector were expressed by the same B-cell. Where (A) The vector encoding for dIgA 1 or dIgA2 comprising in the 5′ to 3′ direction a promoter operably linked to all transgenes expressed as a single open reading frame where each transgene is separated from the subsequent transgene in the 5′ to 3′ direction by (1) a furin cleavage site, (2) a sequence encoding 2A self-processing cleavage site (B) The vector encoding for dIgA1 or dIgA2 comprising in the 5′ to 3′ direction the use of separate promoters and regulatory elements for each transgene (C) The vector encoding for dIgA 1 or dIgA2 comprising the 5′ to 3′ direction a promoter operably linked to three or four transgenes where first transgene is separated from the second transgene in the 5′ to 3′ direction by a (1) furin cleavage site, (2) a sequence encoding 2A self-processing cleavage site where the second transgene has a stop codon and (3) in the 5′ to 3′ direction is followed by an internal ribosome entry site (IRES), J Chain and optionally followed by (IRES) and MZB1 and a polyadenylation element. The optional incorporation of a Woodchuck hepatitis virus post-transcriptional regulatory element (WPRE) to immediately precede any polyadenylation element (D) The mRNA vectors encoding for each of the immunoglobulin heavy chain, immunoglobulin light chain and J Chain as two or three separate vectors intended to be contained together in a single vehicle such as a vesicle or lipid nano particle.

3. An mRNA, viral vector, non-viral vector or retroviral vector coding for any one of IgG1, IgG2, IgG3, IgA1 or IgA2 where the vector contains in any order the transgenes for (1) the immunoglobulin heavy chain IgH, the immunoglobulin light chain that may be kappa (IgLκ) or lambda (IgLλ) as determined from gene sequencing of the cell of interest where (A) The vector comprising in the 5′ to 3′ direction a promoter operably linked to the two transgenes expressed as a single open reading frame where each transgene is separated from the subsequent transgene in the 5′ to 3′ direction by (1) a furin cleavage site, (2) a sequence encoding a 2A self-processing cleavage site. (B) The vector comprising in the 5′ to 3′ direction the use of separate promoters and polyadenylation elements for each transgene with the optional use of a woodchuck hepatitis virus posttranscriptional regulatory element (WPRE) to precede one or more polyadenylation elements.

4. Construction of viral vectors, retroviral vectors, non-viral vectors or mRNA vectors in one or more of claims 1, 2 and 3 wherein the vector is selected from one or more of the group consisting of an adeno-associated virus (AAV) viral vector, an AAV vector, an adenovirus viral vector, a self-inactivating replication-incompetent lentivirus retroviral vector, a self-inactivating replication-incompetent lentivirus viral vector, a self-inactivating lentivirus vector, a non-viral vector, an mRNA vector.

5. Delivery of mRNA, viral vector, non-viral vector or retroviral vector in one or more of claim 1, 2, 3, 4, 9, 10, 11, 12, 15, or 16, with an AAV capsid, a self-inactivating integration-deficient lentivirus, a self-inactivating integration competent lentivirus, a pseudotyped lentivirus, a vesicle based delivery system, a lipid nanoparticle, or as a naked vector via electroporation.

6. The vector according to claims 1, 2, 3, 9, 10 and 11 where the sequence encoding the furin cleavage site encodes an oligopeptide with the consensus sequence from a group consisting of RXK(R)R (SEQ ID NO: 12), RXRYKR (SEQ ID NO: 13), RXRFKR (SEQ ID NO: 14)

7. The vector according to claims 1, 2, 3, 9, 10, 11, 12, 14, 15 and 16 where the 2A self-processing cleavage site is from a group consisting of (SEQ ID NO: 15), (SEQ ID NO: 17) or (SEQ ID NO: 19).

8. Administration of the mRNA, viral, non-viral or retroviral vectors in any of claims 1, 2, 3, 4, 5, 9, 10, 11, 12, 13, 14, 15, 16 to one or more of an animal and/or a human via intramuscular administration to skeletal muscle, intramuscular administration to skeletal muscle with the use of electroporation, intravenous administration, tissue specific administration proximal to a supporting lymph node, direction injection or micro injection into the lamina propria of the stomach, direct injection into the lamina propria of the small intestine, direct injection into the lamina propria of the trachea or bronchi which may be administered with an endoscope or administration proximal to lymph nodes. Optional, one or more additions of target antigens or target proteins or mRNA encoding for them to activate B-cells that received the vectors.

9. Episomal, genomic integrated lentiviral vector or mRNA expression of monoclonal or polyclonal antibodies (immunoglobulins) whose polypeptide sequence for V_{H} and V_{L} immunoglobulin light and heavy chains are determined and identified from claim 1 where IgG and IgA memory B-cells are derived from the blood of persons or animals who are currently infected with or were previously infected with, exposed to, has immune specificity to, or affected by one or more of (1) a virus (s) (2) a systemic ailment such as allergies (3) Allergens (4) fungi (5) bacterial infection (6) cancerous tumor (7) an unnatural virus or toxin (8) any ailment (9) a target protein or variant (10) an immune system protein such as Immunoglobulin class E (IgE) or a cytokine, are modified in the following way: V-regions (both V_{L} and V_{H}) are coded for exactly as they were identified from the source in the cell expressing the potent immunoglobulin or one or more of V_{L} and V_{H} may optionally have one or more of the Complementary Determining Regions (CDR) or Framework regions (FR) modified. Where one or more of the domains of the immunoglobulin heavy chain constant domains consisting of C_{H}1, hinge, C_{H}2 and C_{H}3 are replaced by one or more of (1) natural human derived constant regions to reduce immunogenicity and/or modulate effector functions, (2) engineered constant regions to modulate effector functions and (3) adding a furin cleavage site residue to the C-terminal end of the immunoglobulin heavy chain. The immunoglobulin light chain's (IgL) constant regions (C_{L}) is optionally added or modified by one of more of (4) changing type e.g. kappa (κ) to lambda (λ) or lambda (λ) to kappa (κ), (5) adding a furin cleavage site residue on the C-terminal end, (6) modifying the hinge length, (7) modifying the hinge amino acids or amorphous chain amino acids. Where the dIgA immunoglobulins use immunoglobulins heavy and light chains identified from a single IgA and through incorporating J Chain into the vector where J chain may optionally be modified by adding to its C-terminal end a furin cleavage site residue.

10. Episomal, genomic integrated lentiviral vector or mRNA expression of monoclonal or polyclonal antibodies (immunoglobulins) whose polypeptide sequence for V_{H} and V_{L} immunoglobulin light and heavy chains are determined and identified from claim 1 or from a previously identified potent immunoglobulin where IgG and IgA memory B-cells from claim 1 are derived from the blood of persons or animals who are currently infected with or were previously infected with, exposed to or affected by one or more of ((1) a virus (s) (2) a systemic ailment such as but not limited to allergies (3) Allergens (4) fungi (5) bacterial infection (6) cancerous tumor (7) an unnatural virus or toxin (8) any ailment (9) a target protein or variant (10) an immune system protein such as Immunoglobulin class E (IgE) or a cytokine, are modified in the following way. An IgG1, IgG3, IgA1, IgA2 dIgA2 of dIgA2 immunoglobulin identified to be of moderate to high association constant against the protein of interest may be modified such that such as by engineering the constant domains to minimize Fcγ receptor binding or with mixes of two constant regions from two isotypes or subclass that may be accomplished with (C) combinations of Fab—as identified from the memory B-cell where the dIgA1 may be optionally modified on the immunoglobulin light chain by adding to its C-terminal end a furin cleavage site residue as a result of a byproduct of furin cleavage—where Fc domains are replaced with IgG2 Fc domains or engineered Fc domains or (D) F(ab′)2—as identified from (A) or (B) where the F(ab′)2 of dIgA1 may be optionally modified on the immunoglobulin light chain by adding to its C-terminal end a furin cleavage site residue as a result of a byproduct of furin cleavage. Where the dIgA immunoglobulins are expressed through incorporating, the immunoglobulin heavy and light chains of dIgA, J chain into the vector and optionally MZB1.

11. Episomal, genomic integrated lentiviral vector or mRNA expression of polyclonal or monoclonal antibodies (immunoglobulins) based on one or more of the dIgA 1 and dIgA2 where both of the V_{H} and V_{L} regions or the antibody binding fragment (Fab) are identified or derived from single chain variable fragments (scF_{V}) or Fab from combinatorial libraries assessed by phage display technology. Where scFᵥ used to identify V_{H} and V_{L} used for the formation in one or more of dIgA1 and/or dIgA2 produced by random recombination and shuffling with optional mutagenesis of human V_{H} and V_{L} regions of scFv from human antibody libraries derived different human B-cells including, naïve B-cells, memory B-cells and even plasma secreting B-cells where cells may be derived from the blood of humans of that recovered from the virus of interest or from another human source or from mice with humanized immune systems where the potent single chain variable fragment fragments expressed in antibody libraries are used to identify potent immunoglobulin V_{H} and V_{L} regions pairs that may be used to recombine the V_{L} with the constant regions of the immunoglobulin light chain (IgLκ) or (IgLλ) and combining the V_{H} regions with any of the constant region of IgA1 and IgA2 and their engineered variants, including modified hinge variants that may be used to reduce immunogenicity to produce engineered dimeric immunoglobulins of one or more of dIgA 1 and dIgA2. Where dIgA 1 and dIgA2 will be produced from the dimerization of an IgA1 and IgA2 respectively from their co-expression with J-chain and optionally MZB1 in the same vector. Where up to one or more of IgL, IgH, J-chain and MZB1 may be optionally modified by adding to their C-terminal ends a furin cleavage site residue as a result of a byproduct of furin cleavage. Where MZB1 may be optionally modified by adding to its N-terminal end a 2A self-cleaving peptide residue.

12. Modification of dIgA1 or dIgA2 as defined in claim 11 whose V-regions V_{H} and V_{L} are derived from single chain scFv variable fragments derived phage display technology and subsequent mutagenesis to modulate effector functions or reduce antibody-dependent enhance of infection. The constant regions of such dIgA1 and dIgA2 antibodies may be modified such (A) that one or more of the C_{H}1, hinge, C_{H}2 or C_{H}3 domains of the immunoglobulin heavy chain may be modified to modulate effector functions, reduce antibody dependent enhancement of infection, increase half-life, or modify flexibility between the Fc and the Fab afforded by the hinge amino acids and (B) optional modification of the C_{L} domain.

13. Construction of viral vectors, retroviral vectors, episomal vectors or mRNA vectors in one or more of claims 9, 10, 11, 12, 14 and 15 wherein the vector is selected from one or more of the group consisting of an adeno-associated virus (AAV) viral vector, an AAV vector, an adenovirus viral vector, a self-inactivating replication-incompetent lentivirus retroviral vector, a self-inactivating replication-incompetent lentivirus viral vector, a self-inactivating lentivirus vector, a non-viral vector, an mRNA vector.

14. The vector according to claims 1, 2, 3, 9, 10, 11, 12, 15 and 16 wherein the promoter and intermediate promoter is selected from the group consisting of an elongation factor 1-alpha promoter (EF1α) promoter, a phosphoglycerate kinase-1 promoter (PGK) promoter, a human cytomegalovirus immediate early gene promoter (CMV), an internal ribosome entry site (IRES) substitution for an intermediate promoter that has a similar function to an intermediate promoter, a chimeric liver specific promoter (LSP), a cytomegalovirus enhancer/chicken beta-actin promoter (CAG), a tetracycline responsive promoter (TRE), a transthyretin promoter (TTR), a simian virus 40 promoter (SV40), a CK6 promoter and a RNA polymerase III (Pol III) promoter, the natural promoter established for any gene highly expressed by a cell in humans or highly expressed in the target cell.

15. A viral vector, non-viral vector or retroviral vector coding for one or more of dimeric immunoglobulin A1 (dIgA1) and/or dIgA2 in claims 9, 10, 11, and 12 where the viral vector, non-viral vector or retroviral vector contains the transgenes in any order for (1) the immunoglobulin heavy chain of isotype A1 (IgHA1), the immunoglobulin light chain that may be kappa (IgLκ) or lambda (IgLλ) as determined from gene sequencing of the B-cell of interest in claim 1 and J chain or (2) the immunoglobulin heavy chain of isotype A1 (IgHA1), the immunoglobulin light chain that may be kappa (IgLκ) or lambda (IgLλ) as determined from gene sequencing of the B-cell of interest in claim 1, J chain and MZB1. Where the immunoglobulin light and heavy chains encoded for in any vector were expressed by the same B-cell. Where (A) The vector encoding for dIgA1 comprising in the 5′ to 3′ direction a promoter operably linked to all transgenes expressed as a single open reading frame where each transgene is separated from the subsequent transgene in the 5′ to 3′ direction by (1) a furin cleavage site, (2) a sequence encoding 2A self-processing cleavage site. (B) The vector encoding for dIgA1 comprising in the 5′ to 3′ direction the use of separate promoters and regulatory elements for each transgene with the optional the use of an internal ribosome entry site (IRES) between two transgenes in place of a promoter. (C) The vector encoding for dIgA1 comprising the 5′ to 3′ direction a promoter operably linked to three transgenes where first transgene is separated from the subsequent transgene in the 5′ to 3′ direction by a furin cleavage site, a sequence encoding 2A self-processing cleavage site where the second transgene has a stop codon and in tine 5′ to 3′ direction is followed by an internal ribosome entry site (IRES), the third transgene and a polyadenylation element.

16. A viral vector, non-viral vector or retroviral vector coding for any of IgG1, IgG3, IgG3 and IgA1 as it is described in claims 9 and 10 where the viral vector, non-viral vector or retroviral vector contains the transgenes for (1) immunoglobulin heavy chain (IgH) and immunoglobulin light chain that may be kappa or lambda as determined from gene sequencing of the B-cell of interest in claim 9 or 10 (IgLλ) or (IgLλ). Where (A) The vector comprising in the 5′ to 3′ direction a promoter operably linked to all transgenes expressed as a single open reading frame where each transgene is separated from the subsequent transgene in the 5′ to 3′ direction by (1) a furin cleavage site, (2) a sequence encoding 2A self-processing cleavage site. (B) The vector comprising in the 5′ to 3′ direction the use of separate promoters and regulatory elements for each transgene (C) The mRNA vectors encoding for each of the immunoglobulin heavy chain, immunoglobulin light chain and J chain as two or three separate vectors.

17. The vector according to claims 1, 2, 3, 4, 9, 10, 11, 12, 14, 15 and 16 wherein (A) the intermediate promoter is selected from the group consisting of an elongation factor 1-alpha promoter (EF1α), or internal ribosome entry site (IRES) promoter substitute, a human cytomegalovirus immediate early gene promoter (CMV), a chimeric liver specific promoter (LSP), a cytomegalovirus enhancer/chicken beta-actin promoter (CAG) and a simian virus 40 promoter (SV40). (B) the polyadenylation site is selected from a group consisting of simian virus 40 polyadenylation site (SV40 polyA) and Bovine Growth Hormone polyadenylation site (BGH polyA) and (C) The optional use of one or more Woodchuck hepatitis virus posttranscriptional regulatory elements (WPRE)

18. Any combination of one of more of claims 1, 2, 3, 9, 10, 11, 12, 14, 15, 16 and 21. That is polyclonal expression of immunoglobulins may consist of a mix of naturally identified immunoglobulins, artificially modified immunoglobulins and engineered immunoglobulins.

19. The vector according to claims 9, 10, 11, 12, 14, 15 and 16 where the sequence encoding the furin cleavage site encodes an oligopeptide with the consensus sequence from a group consisting of RXKRR (SEQ ID NO: 12), RXRYKR (SEQ ID NO: 13), RXRFKR (SEQ ID NO: 14)

20. The vectors according to claims 2, 3, 9, 10, 11, 12, 14, 15, and 16 with any unnatural 5′ UTR and 3′ UTR or any natural 5′ UTR and 3′ UTR used by humans is substituted in for one or more of the natural 5′ UTR and 3′ UTR normally transcribed with the gene where the 5′ UTR is placed directly before a transgene with a start codon to start translation and promoter that is not an IRES and the 3′ UTR is placed directly following any transgene with a stop codon that is directly followed by either of a WPRE or polyadenylation element.

21. Delivery of a vaccine including (A) target proteins such as antigens or variants with optional use of adjuvants or (B) mRNA encoding for target protein or variants with optional use of adjuvants via administration via injection or microinjection to the lamina propria of part of the respiratory tract such as the trachea or bronchi or Gastrointestial tract to produce potent dIgA1 and dIgA2 based immunity in addition to other immunoglobulin classes that is both at a systems level but also localized to the organ of interest.

22. The stepwise safety evaluation of the dIgA1 gene therapy by evaluating the individuals suitability for the gene therapy both for moderate term and long term use by sequentially administering two or more of the following (A) One or more administrations of mRNA that is equivalent in encoded proteins to one or more of the DNA-based gene therapies (B) Administration of the integration incompetent lentivirus based delivery system (C) Administration of any non-integrating DNA based viral delivery system. (D) Administration of an integration-competent lentivirus based delivery system.

23. The vectors and immunoglobulins according to claims 2, 3, 9, 10, 11, 15, and 16 where (A) any vector construct has optionally excluded one or more furin cleavage sequences leaving only 2A self-processing peptide sequences between two or more consecutive transgenes that are part of a single open reading frame and (B) one or more of IgH, IgL or J Chain that is modified by adding to their C-terminal ends a 2A self-processing peptide residue as a result of a byproduct 2A cleavage.


SEQUENCE LISTING (103 sequences)

SEQ ID NO: 1 - DNA, 589 bp, Artificial
  Vector Element
  Misc._Feature (1)..(589): Name/Key: Human Cytomegalovirus Immediate Early Enhancer/Promoter (CMV)
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatc

SEQ ID NO: 2 - DNA, 588 bp, Artificial
  Vector Element
  Misc._Feature (1)..(588): Name/Key: Internal Ribosome Entry Site (IRES)
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacc

SEQ ID NO: 3 - DNA, 598 bp, Artificial
  Vector Element
  Misc._Feature (1)..(598): Name/Key: Woodchuck Hepatitis Virus Posttranscriptional Regulatory Element (WPRE)
cgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgg

SEQ ID NO: 4 - DNA, 236 bp, Artificial
  Vector Element
  Misc._Feature (1)..(236): Name/Key: Simian Virus 40 Polyadenylation Signal (SV40 Poly A)
agctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatgg

SEQ ID NO: 5 - DNA, 208 bp, Artificial
  Vector Element
  Misc._Feature (1)..(208): Name/Key: Bovine Growth Hormoen Polyadenylation Signal (BGH Poly A)
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 6 - skipped

SEQ ID NO: 7 - protein, 136 aa, Human
  Misc._Feature (1)..(136): Name/Key: J Chain Without Signaling Peptide
EDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 8 - protein, 189 aa, Human
  Misc._Feature (1)..(189): Name/Key: Marginal Zone B-1 Cell Specific Protein (MZB1)
MRLSLPLLLLLLGAWAIPGGLGDRAPLTATAPQLDDEEMYSAHMPAHLRCDACRAVAYQMWQNLAKAETKLHTSNSGGRRELSELVYTDVLDRSCSRNWQDYGVREVDQVKRLTGPGLSEGPEPSISVMVTGGPWPTRLSRTCLHYLGEFGEDQIYEAHQQGRGALEALLCGGPQGACSEKVSATREEL

SEQ ID NO: 9 - DNA, 117 bp, Artificial
  Vector Element
  Misc._Feature (1)..(117): Name/Key: Intron I117 5′ Untranslated Region (5′ UTR)
gtaagtgatatctactagatttatcaaaaagagtgttgacttgtgagcgctcacaattgatacttagattcatcgagagggacacgtcgactactaaccttcttctctttcctacag

SEQ ID NO: 10 - protein, 4 aa, Artificial
  Vector Element
  Misc._Feature (1)..(4): Name/Key: Consensus Sequence of Furin Cleavage Site (Furin Cleavage Site)
  Misc._Feature (2)..(2): Xaa = any amino acid
RXKR

SEQ ID NO: 11 - protein, 159 aa, Human
  Misc._Feature (1)..(159): Name/Key: J Chain with Signaling Peptid
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 12 - protein, 5 aa, Artificial
  Vector Element
  Misc._Feature (1)..(5): Name/Key: Consensus Sequence of Furin Cleavage Site 2 (Furin Cleavage Site)
  Misc._Feature (2)..(2): Xaa = any amino acid
RXKRR

SEQ ID NO: 13 - protein, 6 aa, Artificial
  Vector Element
  Misc._Feature (1)..(6): Name/Key: Consensus Sequence of Furin Cleavage Site 3 (Furin Cleavage Site)
  Misc._Feature (2)..(2): Xaa = any amino acid
RXRYKR

SEQ ID NO: 14 - protein, 6 aa, Artificial
  Vector Element
  Misc._Feature (1)..(6): Name/Key: Consensus Sequence of Furin Cleavage Site 4 (Furin Cleavage Site)
  Misc._Feature (2)..(2): Xaa = any amino acid
RXRFKR

SEQ ID NO: 15 - protein, 25 aa, Artificial
  Vector Element
  Misc._Feature (1)..(25): Name/Key: Foot and Mouth Disease Virus 2A Self Cleaving/Self Processing Peptide (F2A) or (2A)
GSGVKQTLNFDLLKLAGDVESNPGP

SEQ ID NO: 16 - protein, 19 aa, Artificial
  Vector Element
  Misc._Feature (1)..(19): Name/Key: Porcine Teschovirus 2A Self Cleaving/Self Processing Peptide (P2A) or (2A)
ATNFSLLKQAGDVEENPGP

SEQ ID NO: 17 - protein, 18 aa, Artificial
  Vector Element
  Misc._Feature (1)..(18): Name/Key: Thosea Asigna Virus-2A Self Cleaving/Self Processing Peptide (T2A) or (2A)
EGRGSLLTCGDVEENPGP

SEQ ID NO: 18 - skipped

SEQ ID NO: 19 - protein, 20 aa, Artificial
  Vector Element
  Misc._Feature (1)..(20): Name/Key: Equine Rhinitis A Virus-2A Self Cleaving/Self Processing Peptide (E2A) or (2A)
QCTNYALLKLAGDVESNPGP

SEQ ID NO: 20 - DNA, 278 bp, Artificial
  Vector Element
  Misc._Feature (1)..(278): Name/Key: Chicken Beat Actin Promoter (CAG)
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 21 - DNA, 6 bp, Human
  Misc._Feature (1)..(6): From Page 88 of the Specification
  Misc._Feature (1)..(6): Name/Key: Kozak Sequence
gccacc

SEQ ID NO: 22 - skipped

SEQ ID NO: 23 - protein, 353 aa, Human
  Misc._Feature (1)..(353): Name/Key: Immunoglobulin Heavy Chain Constant Alpha 1 (IgHCA1 Constant Region)
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 24 - protein, 340 aa, Human
  Misc._Feature (1)..(340): Name/Key: Immunoglobulin Heavy Chain Constant Alpha 2 Allotype A2m (IgHCA2 Constant Region Allotype A2m)
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 25 - protein, 106 aa, Human
  Misc._Feature (1)..(1): Xaa = any amino acid
  Misc._Feature (1)..(106): Name/Key: (IgLC_Lambda) Immunoglobuin Lambda Light Chain Constant Region
XQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 26 - protein, 107 aa, Human
  Misc._Feature (1)..(107): Name/Key: (IgLC_Kappa_Km(1)) Immunoglobuin Kappa Light Chain Constant Region Allotype Km(1)
  Misc._Feature (1)..(1): Xaa = any amino acid
  Misc._Feature (56)..(56): Allotype Identifier
  Misc._Feature (94)..(94): Allotype Identifier
XTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 22 aa, Human
  Misc._Feature (1)..(22): Name/Key: Signaling Peptide for J Chain
MKNHLLFWGVLAVFIKAVHVKA

SEQ ID NO: 28 - protein, 19 aa, Human
  Misc._Feature (1)..(19): Name/Key: Signaling Peptide for the Immunoglobulin Heavy Chain (Seq. ID 28)
  Misc._Feature (1)..(19): Most Common in Population
MDWTWRILFLVAAATGAHS

SEQ ID NO: 29 - protein, 19 aa, Human
  Misc._Feature (1)..(19): Name/Key: Signaling Peptide for the Immunoglobulin Heavy Chain (Seq. ID 29)
MDTLCSTLLLLTIPSWVLS

SEQ ID NO: 30 - protein, 19 aa, Human
  Misc._Feature (1)..(19): Name/Key: Signaling Peptide for the Immunoglobulin Kappa Light Chain (Seq. ID 30)
  Misc._Feature (1)..(19): Most Common in Population
MDMRVPAQLLGLLLLWLPG

SEQ ID NO: 31 - protein, 19 aa, Human
  Misc._Feature (1)..(19): Name/Key: Signaling Peptide for the Immunoglobulin Kappa Light Chain (Seq. ID 31)
MDMMVPAQLLGLLLLWFPG

SEQ ID NO: 32 - protein, 20 aa, Human
  Misc._Feature (1)..(20): Name/Key: Signaling Peptide for the Immunoglobulin Kappa Light Chain (Seq. ID 32)
MRVPAQLLGLLLLWLPGARC

SEQ ID NO: 33 - protein, 19 aa, Human
  Misc._Feature (1)..(19): Name/Key: Signaling Peptide for the Immunoglobulin Kappa Light Chain (Seq. ID 33)
MRLPAQLLGLLMLWVPGKD

SEQ ID NO: 34 - protein, 19 aa, Human
  Misc._Feature (1)..(19): Name/Key: Signaling Peptide for the Immunoglobulin Kappa Light Chain (Seq. ID 34)
MRLLAQLLGLLMLWVPGSS

SEQ ID NO: 35 - protein, 19 aa, Human
  Misc._Feature (1)..(19): Name/Key: Signaling Peptide for the Immunoglobulin Lambda Light Chain (Seq. ID 35)
MAWTPLWLTLLTLCIGSVV

SEQ ID NO: 36 - protein, 19 aa, Human
  Misc._Feature (1)..(19): Name/Key: Signaling Peptide for the Immunoglobulin Lambda Light Chain (Seq. ID 36)
MAWTPLFLFLLTCCPGSNS

SEQ ID NO: 37 - protein, 19 aa, Human
  Misc._Feature (1)..(19): Name/Key: Signaling Peptide for the Immunoglobulin Lambda Light Chain (Seq. ID 37)
MAWALLLLTLLTRDTGSWA

SEQ ID NO: 38 - protein, 19 aa, Human
  Misc._Feature (1)..(19): Name/Key: Signaling Peptide for the Immunoglobulin Lambda Light Chain (Seq. ID 38)
MPWALLLLTLLTHSAVSVV

SEQ ID NO: 39 - protein, 19 aa, Human
  Misc._Feature (1)..(19): Name/Key: Signaling Peptide for the Immunoglobulin Lambda Light Chain (Seq. ID 39)
MAWSSLLLTLLAHCTGSWA

SEQ ID NO: 40 - protein, 19 aa, Human
  Misc._Feature (1)..(19): Name/Key: Signaling Peptide for the Immunoglobulin Lambda Light Chain (Seq. ID 40)
MAWSPLFLTLITHCAGSWA

SEQ ID NO: 41 - protein, 19 aa, Human
  Misc._Feature (1)..(19): Name/Key: Signaling Peptide for the Immunoglobulin Lambda Light Chain (Seq. ID 41)
MIYEVSHRPSGVSTRFSAS

SEQ ID NO: 42 - DNA, 18 bp, Human
  Misc._Feature (1)..(18): Name/Key: 5′ Untranslated Region (5′ UTR) of J Chain
agaagaagtgaagtcaag

SEQ ID NO: 43 - DNA, 36 bp, Human
  Misc._Feature (1)..(36): Name/Key: 5′ Untranslated Region (5′ UTR) of the Immunoglobulin Lambda Light Chain (Seq. ID 43)
tgagcgcagaaggcaggactcgggacaatcttcatc

SEQ ID NO: 44 - DNA, 40 bp, Human
  Misc._Feature (1)..(40): Name/Key: 5′ Untranslated Region (5′ UTR) of the Immunoglobulin Lambda Light Chain (Seq. ID 44)
gctgcgggtagagaagacaggactcaggacaatctccagc

SEQ ID NO: 45 - DNA, 29 bp, Human
  Misc._Feature (1)..(29): Name/Key: 5′ Untranslated Region (5′ UTR) of the Immunoglobulin Kappa Light Chain (Seq. ID 45)
gcaggaatcagtcccactcaggacacagc

SEQ ID NO: 46 - DNA, 53 bp, Human
  Misc._Feature (1)..(53): Name/Key: 5′ Untranslated Region (5′ UTR) of the Immunoglobulin Kappa Light Chain (Seq. ID 46)
aggctggacacacttcatgcaggagtcagaccctgtcaggacacagcatagac

SEQ ID NO: 47 - DNA, 63 bp, Human
  Misc._Feature (1)..(63): Name/Key: 5′ Untranslated Region (5′ UTR) of the Immunoglobulin Heavy Chain (Seq. ID 47)
gagagcatcacccagcaaccacatctgtcctctagagatcccctgagagctccgttcctcacc

SEQ ID NO: 48 - DNA, 20 bp, Human
  Misc._Feature (1)..(20): Name/Key: 5′ Untranslated Region (5′ UTR) of the Immunoglobulin Heavy Chain (Seq. ID 48)
agtgactcctgtgccccacc

SEQ ID NO: 49 - DNA, 37 bp, Human
  Misc._Feature (1)..(37): Name/Key: 5′ Untranslated Region (5′ UTR) of the Hemoglobin Subunit Alpha 1
actcttctggtccccacagactcagagagaacccacc

SEQ ID NO: 50 - DNA, 37 bp, Human; identical to SEQ ID NO: 49
  Misc._Feature (1)..(37): Name/Key: 5′ Untranslated Region (5′ UTR) of the Hemoglobin Subunit Alpha 2

SEQ ID NO: 51 - DNA, 47 bp, Human
  Misc._Feature (1)..(47): Name/Key: 5′ Untranslated Region (5′ UTR) of the Fibrinogen-Gamma Chain
gacagtgctgacactacaaggctcggagctccgggcactcagacatc

SEQ ID NO: 52 - DNA, 8 bp, Human
  Misc._Feature (1)..(8): Name/Key: 5′ Untranslated Region (5′ UTR) of the Fibrinogen-Beta Chain
aagtctac

SEQ ID NO: 53 - DNA, 55 bp, Human
  Misc._Feature (1)..(55): Name/Key: 5′ Untranslated Region (5′ UTR) of the Fibrinogen-Alpha Chain
aatcctttctttcagctggagtgctcctcaggagccagccccacccttagaaaag

SEQ ID NO: 54 - DNA, 41 bp, Human
  Misc._Feature (1)..(41): Name/Key: 5′ Untranslated Region (5′ UTR) for Albumin
ctagcttttctcttctgtcaaccccacacgcctttggcaca

SEQ ID NO: 55 - DNA, 788 bp, Human
  Misc._Feature (1)..(788): Name/Key: 3′ Untranslated Region (3′ UTR) of J Chain
tttaagtcattgctgactgcatagctctttttcttgagaggctctccattttgattcagaaagttagcatatttattaccaatgaatttgaaaccagggcttttttttttttttgggtgatgtaaaaccaactccctgccaccaaaataattaaaatagtcacattgttatctttattaggtaatcacttcttaattatatgttcatactctaagtatcaaaatcttccaattatcatgctcacctgaaagaggtatgctctcttaggaatacagtttctagcattaaacaaataaacaaggggagaaaataaaactcaaggactgaaaatcaggaggtgtaataaaatgttcctcgcattcccccccgcttttttttttttttttgactttgccttggagagccagagcttccgcattttctttactattctttttaaaaaaagtttcactgtgtagagaacatatatgcataaacataggtcaattatatgtctccattagaaaaataataattggaaaacatgttctagaactagttacaaaaataatttaaggtgaaatctctaatatttataaaagtagcaaaataaatgcataattaaaatatatttggacataacagacttggaagcagatgatacagacttctttttttcataatcaggttagtgtaagaaattgccatttgaaacaatccattttgtaactgaaccttatgaaatatatgtatttcatggtacgtattctctagcacagtctgagcaattaaatagattcataagcata

SEQ ID NO: 56 - DNA, 135 bp, Human
  Misc._Feature (1)..(135): Name/Key: 3′ Untranslated Region (3′ UTR) of the Immunoglobulin Heavy Chain Class G Subclass 1 (IgHG1)
gtgccacggccggcaagcccccgctccccaggctctcggggtcgcgcgaggatgcttggcacgtaccccgtgtacatacttcccaggcacccagcatggaaataaagcacccagcgcttccctgggcccctgcga

SEQ ID NO: 57 - DNA, 135 bp, Human; identical to SEQ ID NO: 56
  Misc._Feature (1)..(135): Name/Key: 3′ Untranslated Region (3′ UTR) of the Immunoglobulin Heavy Chain Class G Subclass 2 (IgHG2)

SEQ ID NO: 58 - DNA, 135 bp, Human; identical to SEQ ID NO: 56
  Misc._Feature (1)..(135): Name/Key: 3′ Untranslated Region (3′ UTR) of the Immunoglobulin Heavy Chain Class G Subclass 3 (IgHG3)

SEQ ID NO: 59 - DNA, 135 bp, Human; identical to SEQ ID NO: 56
  Misc._Feature (1)..(135): Name/Key: 3′ Untranslated Region (3′ UTR) of the Immunoglobulin Heavy Chain Class G Subclass 4 (IgHG4)

SEQ ID NO: 60 - DNA, 113 bp, Human
  Misc._Feature (1)..(113): Name/Key: 3′ Untranslated Region (3′ UTR) of the Immunoglobulin Heavy Chain Class A Subclass 1 (IgHA1) for B-cell Receptor
gcaggagccggcaaggcacagggaggaagtgtggaggaacctcttggagaagccagctatgcttgccagaactcagccctttcagacatcaccgacccgcccttactcacatg

SEQ ID NO: 61 - DNA, 50 bp, Human
  Misc._Feature (1)..(50): Name/Key: 3′ Untranslated Region (3′ UTR) of the Immunoglobulin Heavy Chain Class A Subclass 1 (IgHA1)
gccgcccgcctgtccccacccctgaataaactccatgctcccccaagcag

SEQ ID NO: 62 - DNA, 144 bp, Human
  Misc._Feature (1)..(144): Name/Key: 3′ Untranslated Region (3′ UTR) of the Immunoglobulin Heavy Chain Class A Subclass 2 (IgHA2) for B-cell Receptor
gcgggagccggcaaggcacagggaggaagtgtggaggaacctcttggagaagccagctatgcttgccagaactcagccctttcagacatcaccgacccgcccttactcacgtggcttccaggtgcaataaagtggccccaagga

SEQ ID NO: 63 - DNA, 48 bp, Human
  Misc._Feature (1)..(48): Name/Key: 3′ Untranslated Region (3′ UTR) of the Immunoglobulin Heavy Chain Class A Subclass 2 (IgHA2)
gccgcccgcctgtccccacccctgaataaactccatgctcccccaagc

SEQ ID NO: 64 - DNA, 140 bp, Human
  Misc._Feature (1)..(140): Name/Key: 3′ Untranslated Region (3′ UTR) of the Immunoglobulin Kappa Light Chain (IgKappaL)
gttcccaactctaaccccacccacgggagcctggagctgcaggatcccaggggaggggtctctctccccatcccaagtcatccagcccttctccctgcactcatgaaaccccaataaatatcctcattgacaaccagaaa

SEQ ID NO: 65 - DNA, 140 bp, Human; identical to SEQ ID NO: 64
  Misc._Feature (1)..(140): Name/Key: 3′ Untranslated Region (3′ UTR) of the Immunoglobulin Lambda Light Chain (IgLambdaL)

SEQ ID NO: 66 - DNA, 111 bp, Human
  Misc._Feature (1)..(111): Name/Key: 3′ Untranslated Region (3′ UTR) of the Hemoglobulin Subunit Alpha 1 (HBA1)
gctggagcctcggtggccatgcttcttgccccttgggcctccccccagcccctcctccccttcctgcacccgtacccccgtggtctttgaataaagtctgagtgggcggca

SEQ ID NO: 67 - DNA, 110 bp, Human
  Misc._Feature (1)..(110): Name/Key: 3′ Untranslated Region (3′ UTR) of the Hemoglobulin Subunit Alpha 2 (HBA2)
gctggagcctcggtagccgttcctcctgcccgctgggcctcccaacgggccctcctcccctccttgcaccggcccttcctggtctttgaataaagtctgagtgggcagca

SEQ ID NO: 68 - DNA, 232 bp, Human
  Misc._Feature (1)..(232): Name/Key: 3′ Untranslated Region (3′ UTR) of the Fibrinogen-Gamma Chain
aaaattatgtctttttaatatggtttttgttttgttatatattcacaggctggagacgtttaaaagaccgtttcaaaagagatttacttttttaaaggactttatctgaacagagagatataatatttttcctattggacaatggacttgcaaagcttcacttcattttaagagcaaaagaccccatgttgaaaactccataacagttttatgctgatgataatttatctac

SEQ ID NO: 69 - DNA, 219 bp, Human
  Misc._Feature (1)..(219): Name/Key: 3′ Untranslated Region (3′ UTR) of the Fibrinogen-Alpha Chain
actaagttaaatatttctgcacagtgttcccatggccccttgcatttccttcttaactctctgttacacgtcattgaaactacacttttttggtctgtttttgtgctagactgtaagttccttgggggcagggcctttgtctgtctcatctctgtattcccaaatgcctaacagtacagagccatgactcaataaatacatgttaaatggatgaatgaa

SEQ ID NO: 70 - DNA, 414 bp, Human
  Misc._Feature (1)..(414): Name/Key: 3′ Untranslated Region (3′ UTR) of Albumin
catcacatttaaaagcatctcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaagttatggattataaacattcaaaataatattttgacattatgataattctgaataaaagaacaaaaacca

SEQ ID NO: 71 - RNA, 588 nt, Artificial
  Vector Element
  Misc._Feature (1)..(588): Single Stranded RNA form of Sequence 2
  Misc._Feature (1)..(588): Name/Key: Internal Ribosome Entry Site (IRES) mRNA
gccccucucccuccccccccccuaacguuacuggccgaagccgcuuggaauaaggccggugugcguuugucuauauguuauuuuccaccauauugccgucuuuuggcaaugugagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacauguguuuagucgagguuaaaaaaacgucuaggccccccgaaccacggggacgugguuuuccuuugaaaaacacgaugauaauauggccacaacc

SEQ ID NO: 72 - RNA, 598 nt, Artificial
  Vector Element
  Misc._Feature (1)..(598): Single Stranded RNA Form of Sequence 3.
  Misc._Feature (1)..(598): Name/Key: Woodchuck Hepatitis Virus Posttranscriptional Regulatory Element (WPRE) mRNA
cgauaaucaaccucuggauuacaaaauuugugaaagauugacugguauucuuaacuauguugcuccuuuuacgcuauguggauacgcugcuuuaaugccuuuguaucaugcuauugcuucccguauggcuuucauuuucuccuccuuguauaaauccugguugcugucucuuuaugaggaguuguggcccguugucaggcaacguggcguggugugcacuguguuugcugacgcaacccccacugguuggggcauugccaccaccugucagcuccuuuccgggacuuucgcuuucccccucccuauugccacggcggaacucaucgccgccugccuugcccgcugcuggacaggggcucggcuguugggcacugacaauuccgugguguugucggggaagcugacguccuuuccauggcugcucgccuguguugccaccuggauucugcgcgggacguccuucugcuacgucccuucggcccucaauccagcggaccuuccuucccgcggccugcugccggcucugcggccucuuccgcgucuucgccuucgcccucagacgagucggaucucccuuugggccgccuccccgcaucgg

SEQ ID NO: 73 - RNA, 6 nt, Human
  Misc._Feature (1)..(6): Single Stranded RNA form of Sequence 21
  Misc._Feature (1)..(6): Name/Key: Kozak Sequence mRNA
gccacc

SEQ ID NO: 74 - RNA, 18 nt, Human
  Misc._Feature (1)..(18): Single Stranded RNA form of Sequence 42.
  Misc._Feature (1)..(18): Name/Key: 5′ Untranslated Region (5′ UTR) of J Chain
agaagaagugaagucaag

SEQ ID NO: 75 - RNA, 36 nt, Human
  Misc._Feature (1)..(36): Single Stranded RNA form of Sequence 43
  Misc._Feature (1)..(36): Name/Key: 5′ Untranslated Region (5′ UTR) of the Immunoglobulin Lambda Light Chain (Seq. ID 75) mRNA
ugagcgcagaaggcaggacucgggacaaucuucauc

SEQ ID NO: 76 - RNA, 40 nt, Human
  Misc._Feature (1)..(40): Single Stranded RNA form of Sequence 44
  Misc._Feature (1)..(40): Name/Key: 5′ Untranslated Region (5′ UTR) of the Immunoglobulin Lambda Light Chain (Seq. ID 76) mRNA
gcugcggguagagaagacaggacucaggacaaucuccagc

SEQ ID NO: 77 - RNA, 29 nt, Human
  Misc._Feature (1)..(29): Single Stranded RNA form of Sequence 45
  Misc._Feature (1)..(29): Name/Key: 5′ Untranslated Region (5′ UTR) of the Immunoglobulin Kappa Light Chain (Seq. ID 77) mRNA
gcaggaaucagucccacucaggacacagc

SEQ ID NO: 78 - RNA, 53 nt, Human
  Misc._Feature (1)..(53): Single Stranded RNA form of Sequence 46
  Misc._Feature (1)..(53): Name/Key: 5′ Untranslated Region (5′ UTR) of the Immunoglobulin Kappa Light Chain (Seq. ID 78) mRNA
aggcuggacacacuucaugcaggagucagacccugucaggacacagcauagac

SEQ ID NO: 79 - RNA, 63 nt, Human
  Misc._Feature (1)..(63): Single Stranded RNA form of Sequence 47; Name/Key: 5′ Untranslated Region (5′ UTR) of the Immunoglobulin Heavy Chain (Seq. ID 79) mRNA
gagagcaucacccagcaaccacaucuguccucuagagauccccugagagcuccguuccucacc

SEQ ID NO: 80 - RNA, 20 nt, Human
  Misc._Feature (1)..(20): Single Stranded RNA form of Sequence 48
  Misc._Feature (1)..(20): Name/Key: 5′ Untranslated Region (5′ UTR) of the Immunoglobulin Heavy Chain (Seq. ID 80) mRNA
agugacuccugugccccacc

SEQ ID NO: 81 - RNA, 37 nt, Human
  Misc._Feature (1)..(37): Single Stranded RNA form of Sequence 49
  Misc._Feature (1)..(37): Name/Key: 5′ Untranslated Region (5′ UTR) of the Hemoglobin Subunit Alpha 1 mRNA
acucuucugguccccacagacucagagagaacccacc

SEQ ID NO: 82 - RNA, 37 nt, Human; identical to SEQ ID NO: 81
  Misc._Feature (1)..(37): Single Stranded RNA form of Sequence 50
  Misc._Feature (1)..(37): Name/Key: 5′ Untranslated Region (5′ UTR) of the Hemoglobin Subunit Alpha 2 mRNA

SEQ ID NO: 83 - RNA, 47 nt, Human
  Misc._Feature (1)..(47): Single Stranded RNA form of Sequence 51
  Misc._Feature (1)..(47): Name/Key: 5′ Untranslated Region (5′ UTR) of the Fibrinogen-Gamma Chain mRNA
gacagugcugacacuacaaggcucggagcuccgggcacucagacauc

SEQ ID NO: 84 - RNA, 8 nt, Human
  Misc._Feature (1)..(8): Single Stranded RNA form of Sequence 52
  Misc._Feature (1)..(8): Name/Key: 5′ Untranslated Region (5′ UTR) of the Fibrinogen-Beta Chain mRNA
aagucuac

SEQ ID NO: 85 - RNA, 55 nt, Human
  Misc._Feature (1)..(55): Single Stranded RNA form of Sequence 53
  Misc._Feature (1)..(55): Name/Key: 5′ Untranslated Region (5′ UTR) of the Fibrinogen-Alpha Chain mRNA
aauccuuucuuucagcuggagugcuccucaggagccagccccacccuuagaaaag

SEQ ID NO: 86 - RNA, 41 nt, Human
  Misc._Feature (1)..(41): Single Stranded RNA form of Sequence 54
  Misc._Feature (1)..(41): Name/Key: 5′ Untranslated Region (5′ UTR) for Albumin mRNA
cuagcuuuucucuucugucaaccccacacgccuuuggcaca

SEQ ID NO: 87 - RNA, 788 nt, Human
  Misc._Feature (1)..(788): Single Stranded RNA form of Sequence 55
  Misc._Feature (1)..(788): Name/Key: 3′ Untranslated Region (3′ UTR) of J Chain mRNA
uuuaagucauugcugacugcauagcucuuuuucuugagaggcucuccauuuugauucagaaaguuagcauauuuauuaccaaugaauuugaaaccagggcuuuuuuuuuuuuuugggugauguaaaaccaacucccugccaccaaaauaauuaaaauagucacauuguuaucuuuauuagguaaucacuucuuaauuauauguucauacucuaaguaucaaaaucuuccaauuaucaugcucaccugaaagagguaugcucucuuaggaauacaguuucuagcauuaaacaaauaaacaaggggagaaaauaaaacucaaggacugaaaaucaggagguguaauaaaauguuccucgcauucccccccgcuuuuuuuuuuuuuuuugacuuugccuuggagagccagagcuuccgcauuuucuuuacuauucuuuuuaaaaaaaguuucacuguguagagaacauauaugcauaaacauaggucaauuauaugucuccauuagaaaaauaauaauuggaaaacauguucuagaacuaguuacaaaaauaauuuaaggugaaaucucuaauauuuauaaaaguagcaaaauaaaugcauaauuaaaauauauuuggacauaacagacuuggaagcagaugauacagacuucuuuuuuucauaaucagguuaguguaagaaauugccauuugaaacaauccauuuuguaacugaaccuuaugaaauauauguauuucaugguacguauucucuagcacagucugagcaauuaaauagauucauaagcaua

SEQ ID NO: 88 - RNA, 135 nt, Human
  Misc._Feature (1)..(135): Single Stranded RNA form of Sequence 56
  Misc._Feature (1)..(135): Name/Key: 3′ Untranslated Region (3′ UTR) of the Immunoglobulin Heavy Chain Class G Subclass 1 (IgHG1) mRNA
gugccacggccggcaagcccccgcuccccaggcucucggggucgcgcgaggaugcuuggcacguaccccguguacauacuucccaggcacccagcauggaaauaaagcacccagcgcuucccugggccccugcga

SEQ ID NO: 89 - RNA, 135 nt, Human; identical to SEQ ID NO: 88
  Misc._Feature (1)..(135): Single Stranded RNA form of Sequence 57
  Misc._Feature (1)..(135): Name/Key: 3′ Untranslated Region (3′ UTR) of the Immunoglobulin Heavy Chain Class G Subclass 2 (IgHG2) mRNA

SEQ ID NO: 90 - RNA, 135 nt, Human; identical to SEQ ID NO: 88
  Misc._Feature (1)..(135): Single Stranded RNA form of Sequence 58
  Misc._Feature (1)..(135): Name/Key: 3′ Untranslated Region (3′ UTR) of the Immunoglobulin Heavy Chain Class G Subclass 3 (IgHG3) mRNA

SEQ ID NO: 91 - RNA, 135 nt, Human; identical to SEQ ID NO: 88
  Misc._Feature (1)..(135): Single Stranded RNA form of Sequence 59
  Misc._Feature (1)..(135): Name/Key: 3′ Untranslated Region (3′ UTR) of the Immunoglobulin Heavy Chain Class G Subclass 4 (IgHG4) mRNA

SEQ ID NO: 92 - RNA, 113 nt, Human
  Misc._Feature (1)..(113): Single Stranded RNA form of Sequence 60
  Misc._Feature (1)..(113): Name/Key: 3′ Untranslated Region (3′ UTR) of the Immunoglobulin Heavy Chain Class A Subclass 1 (IgHA1) for B-cell Receptor mRNA
gcaggagccggcaaggcacagggaggaaguguggaggaaccucuuggagaagccagcuaugcuugccagaacucagcccuuucagacaucaccgacccgcccuuacucacaug

SEQ ID NO: 93 - RNA, 50 nt, Human
  Misc._Feature (1)..(50): Single Stranded RNA form of Sequence 61
  Misc._Feature (1)..(50): Name/Key: 3′ Untranslated Region (3′ UTR) of the Immunoglobulin Heavy Chain Class A Subclass 1 (IgHA1) mRNA
gccgcccgccuguccccaccccugaauaaacuccaugcucccccaagcag

SEQ ID NO: 94 - RNA, 144 nt, Human
  Misc._Feature (1)..(144): Single Stranded RNA form of Sequence 62
  Misc._Feature (1)..(144): Name/Key: 3′ Untranslated Region (3′ UTR) of the Immunoglobulin Heavy Chain Class A Subclass 2 (IgHA2) for B-cell Receptor mRNA
gcgggagccggcaaggcacagggaggaaguguggaggaaccucuuggagaagccagcuaugcuugccagaacucagcccuuucagacaucaccgacccgcccuuacucacguggcuuccaggugcaauaaaguggccccaagga

SEQ ID NO: 95 - RNA, 48 nt, Human
  Misc._Feature (1)..(48): Single Stranded RNA form of Sequence 63
  Misc._Feature (1)..(48): Name/Key: 3′ Untranslated Region (3′ UTR) of the Immunoglobulin Heavy Chain Class A Subclass 2 (IgHA2) mRNA
gccgcccgccuguccccaccccugaauaaacuccaugcucccccaagc

SEQ ID NO: 96 - RNA, 140 nt, Human
  Misc._Feature (1)..(140): Single Stranded RNA form of Sequence 64
  Misc._Feature (1)..(140): Name/Key: 3′ Untranslated Region (3′ UTR) of the Immunoglobulin Kappa Light Chain (IgKappaL) mRNA
guucccaacucuaaccccacccacgggagccuggagcugcaggaucccaggggaggggucucucuccccaucccaagucauccagcccuucucccugcacucaugaaaccccaauaaauauccucauugacaaccagaaa

SEQ ID NO: 97 - RNA, 140 nt, Human; identical to SEQ ID NO: 96
  Misc._Feature (1)..(140): Single Stranded RNA form of Sequence 65
  Misc._Feature (1)..(140): Name/Key: 3′ Untranslated Region (3′ UTR) of the Immunoglobulin Lambda Light Chain (IgLambdaL) mRNA

SEQ ID NO: 98 - RNA, 111 nt, Human
  Misc._Feature (1)..(111): Single Stranded RNA form of Sequence 66
  Misc._Feature (1)..(111): Name/Key: 3′ Untranslated Region (3′ UTR) of the Hemoglobulin Subunit Alpha 1 (HBA1) mRNA
gcuggagccucgguggccaugcuucuugccccuugggccuccccccagccccuccuccccuuccugcacccguacccccguggucuuugaauaaagucugagugggcggca

SEQ ID NO: 99 - RNA, 110 nt, Human
  Misc._Feature (1)..(110): Single Stranded RNA form of Sequence 67
  Misc._Feature (1)..(110): Name/Key: 3′ Untranslated Region (3′ UTR) of the Hemoglobulin Subunit Alpha 2 (HBA2) mRNA
gcuggagccucgguagccguuccuccugcccgcugggccucccaacgggcccuccuccccuccuugcaccggcccuuccuggucuuugaauaaagucugagugggcagca

SEQ ID NO: 100 - RNA, 232 nt, Human
  Misc._Feature (1)..(232): Single Stranded RNA form of Sequence 68
  Misc._Feature (1)..(232): Name/Key: 3′ Untranslated Region (3′ UTR) of the Fibrinogen-Gamma Chain mRNA
aaaauuaugucuuuuuaauaugguuuuuguuuuguuauauauucacaggcuggagacguuuaaaagaccguuucaaaagagauuuacuuuuuuaaaggacuuuaucugaacagagagauauaauauuuuuccuauuggacaauggacuugcaaagcuucacuucauuuuaagagcaaaagaccccauguugaaaacuccauaacaguuuuaugcugaugauaauuuaucuac

SEQ ID NO: 101 - RNA, 219 nt, Human
  Misc._Feature (1)..(219): Single Stranded RNA form of Sequence 69
  Misc._Feature (1)..(219): Name/Key: 3′ Untranslated Region (3′ UTR) of the Fibrinogen-Alpha Chain mRNA
acuaaguuaaauauuucugcacaguguucccauggccccuugcauuuccuucuuaacucucuguuacacgucauugaaacuacacuuuuuuggucuguuuuugugcuagacuguaaguuccuugggggcagggccuuugucugucucaucucuguauucccaaaugccuaacaguacagagccaugacucaauaaauacauguuaaauggaugaaugaa

SEQ ID NO: 102 - RNA, 414 nt, Human
  Misc._Feature (1)..(414): Single Stranded RNA form of Sequence 70
  Misc._Feature (1)..(414): Name/Key: 3′ Untranslated Region (3′ UTR) of Albumin mRNA
caucacauuuaaaagcaucucagccuaccaugagaauaagagaaagaaaaugaagaucaaaagcuuauucaucuguuuuucuuuuucguugguguaaagccaacacccugucuaaaaaacauaaauuucuuuaaucauuuugccucuuuucucugugcuucaauuaauaaaaaauggaaagaaucuaauagagugguacagcacuguuauuuuucaaagauguguugcuauccugaaaauucuguagguucuguggaaguuccaguguucucucuuauuccacuucgguagaggauuucuaguuucuugugggcuaauuaaauaaaucauuaauacucuucuaaguuauggauuauaaacauucaaaauaauauuuugacauuaugauaauucugaauaaaagaacaaaaacca

SEQ ID NO: 103 - RNA, 117 nt, Artificial
  Vector Element
  Misc._Feature (1)..(117): Single Stranded RNA form of Sequence 9
  Misc._Feature (1)..(117): Name/Key: Intron I117 5′ Untranslated Region (5′ UTR) mRNA
guaagugauaucuacuagauuuaucaaaaagaguguugacuugugagcgcucacaauugauacuuagauucaucgagagggacacgucgacuacuaaccuucuucucuuuccuacag